(19) United States
(12) Patent Application Publication
(10) Pub. No.: US 2025/0186419 A1
(43) Pub. Date: Jun. 12, 2025

(54) DOSING REGIMENS FOR TREATING PAIN

(71) Applicant: VERTEX PHARMACEUTICALS INCORPORATED, Boston, MA (US)

(72) Inventors: Radhika KARKARE, Framingham, MA (US); Cathy CHU, Cambridge, MA (US); Brenda CIRINCIONE, Boston, MA (US); Darin J. CORRELL, Dover, MA (US); Philip Kaj Harder DELFF, Watertown, MA (US); Kirk Raymond DINEHART, Holliston, MA (US); James B. JONES, Norfolk, MA (US); Katie L. MCCARTY, Watertown, MA (US); Catherine P. METZLER, Medford, MA (US); Jonathan M. MILLER, Grafton, MA (US); Mark C. PETERSON, Westford, MA (US); Rahul ROOPWANI, Natick, MA (US); John F. STAROPOLI, Boston, MA (US)

(73) Assignee: VERTEX PHARMACEUTICALS INCORPORATED, Boston, MA (US)

(21) Appl. No.: 18/972,540

(22) Filed: Dec. 6, 2024

Related U.S. Application Data
(60) Provisional application No. 63/607,389, filed on Dec. 7, 2023.
(60) Provisional application No. 63/609,206, filed on Dec. 12, 2023.
(60) Provisional application No. 63/625,906, filed on Jan. 26, 2024.
(60) Provisional application No. 63/626,017, filed on Jan. 28, 2024.

(51) Int. Cl. A61K 31/443; A61K 9/20; A61K 9/28; A61P 25/02

(52) U.S. Cl. CPC A61K 31/443; A61K 9/2009; A61K 9/2018; A61K 9/2054; A61K 9/2826; A61K 9/2853; A61K 9/2866; A61P 25/02

(57) ABSTRACT

Disclosed herein are methods of treating pain, comprising administering Compound 1 or a pharmaceutically acceptable salt thereof.

### CROSS-REFERENCE TO RELATED APPLICATIONS

This application claims the benefit of U.S. Provisional Application No. 63/607,387, filed Dec. 7, 2023, U.S. Provisional Application No. 63/609,206, filed Dec. 12, 2023, U.S. Provisional Application No. 63/625,906, filed Jan. 26, 2024, and U.S. Provisional Application No. 63/626,017, filed Jan. 28, 2024, each of which is incorporated by reference in its entirety.

### BACKGROUND

Pain is a protective mechanism that enables healthy persons and animals to avoid tissue damage and to prevent further damage to injured tissue. Managing pain in the clinical setting, in both acute and chronic clinical settings, remains a high unmet need. In addition to acute pain, there are many conditions where chronic pain persists beyond its protective role (neuropathic pain) where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories: pain caused by generalized metabolic damage to the nerve and pain caused by a discrete nerve injury. The metabolic neuropathies include post herpetic neuropathy, diabetic neuropathy, and drug-induced neuropathy. Discrete nerve injuries indications include post amputation pain, post-surgical nerve injury pain, and nerve entrapment injuries like neuropathic back pain. Neuropathic pain is a major cause or disability worldwide, negatively affecting patient's sleep, mood, and functionality. Clin. Ther. 2018 40(6): p. 828-49.

Current pain therapies suffer from poor efficacy and a high risk of adverse events (AEs). For example, lidocaine (a nonselective sodium channel blocker) may effectively reduce pain, but its utility is limited because of prominent side effects when given at dose levels required for pain relief. Opioid pain medications have a high abuse liability, leading to frequent deaths due to overdose. In addition, opioid-induced hyperalgesia also limits the long term use of opioids. Opioid-induced hyperalgesia is encountered regularly in clinical practice and creates significant challenges in pain management.

Antidepressants and anticonvulsants remain the first line treatment for neuropathic pain despite not being designated for such purpose. Their use is often limited by an arsenal of side effects or inadequate pain relief. Clinical development has exhibited a considerable lack of recent progress and innovation of new medications to treat both acute and chronic pain. Over the last decades, most approved analgesic drugs for the treatment of neuropathic pain either act on the serotonin-norepinephrine system (such as the serotonin-norepinephrine reuptake inhibitor duloxetine) or on voltage-gated calcium channels (such as the gabapentinoid pregabalin). Given the limited treatment options for pain, combined with a growing awareness of the risks and relative ineffectiveness of the current standards of care, the development of analgesics targeting specific pathophysiology mechanisms with improved efficacy and safety profiles is vital for better pain management and patient health outcomes.

Voltage-gated sodium channels (Navs) are involved in pain signaling. Navs are biological mediators of electrical signaling as they mediate the rapid upstroke of the action potential of many excitable cell types (e.g., neurons, skeletal myocytes, cardiac myocytes). Support for the assertion that Navs play a critical and central role in pain signaling arises from (1) evaluation of the role Navs plays in normal physiology, (2) pathological states arising from mutations in the Nav1.8 gene (SCN10A). (3) preclinical work in animal models, and (4) pharmacology of known Nav1.8-modulating agents. In addition, because Nav1.8 expression is restricted to peripheral neurons, particularly those that sense pain (e.g., the dorsal root ganglia), Nav1.8 inhibitors are less likely to be associated with the side effects commonly observed with other sodium channel modulators and the abuse liability associated with opioid therapies. Therefore, targeting the underlying biology of pain through selective Nav1.8 inhibition represents a novel approach to analgesic drug development that has the potential to address an urgent unmet need for safe and effective acute and chronic pain therapies (Rush, A. M. and T. R. Cummins, Painful Research: Identification of a Small-Molecule Inhibitor that Selectively Targets Nav.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Because of the role Navs play in the initiation and propagation of neuronal signals, antagonists that reduce Nav currents can prevent or reduce neural signaling and Nav channels have been considered likely targets to reduce pain in conditions where hyper-excitability is observed (Chahine, M., Chatelier, A., Babich, O., and Krupp, J. J., Voltage-gated sodium channels in neurological disorders. CNSNeurol. Disord. Drug Targets 7 (2), p. 144-58 (2008)). Several clinically useful analgesics have been identified as inhibitors of Nav channels. The local anesthetic drugs such as lidocaine block pain by inhibiting Nav channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J. Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Naᵥs form a subfamily of the voltage-gated ion channel super-family and comprises 9 isoforms, designated Nav1.1-Nav1.9. The tissue localizations of the nine isoforms vary. Naᵥ1.4 is the primary sodium channel of skeletal muscle, and Naᵥ1.5 is the primary sodium channel of cardiac myocytes. Naᵥ1.7, Naᵥ1.8 and Naᵥ1.9 are primarily localized to the peripheral nervous system, while Naᵥ1.1, Naᵥ1.2, Naᵥ1.3, and Naᵥ1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Naᵥ1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Naᵥ1.8 has been shown to be a carrier of the sodium current that maintains action potential firing in small DRG neurons, supporting its potential as a target for multiple indications or across multiple pain types (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Naᵥ1.8 is involved in spontaneous firing in damaged neurons, like those that drive neuropathic pain (Roza, C., et al., The tetrodotoxin-resistant Na⁺ channel Naᵥ1.8 is essential for the expression of spontaneous activity in damaged sensory axons of mice. J. Physiol., 2003. 550(Pt 3): p. 921-6; Jarvis, M. F., et al., A-803467, a potent and selective Naᵥ1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Naᵥ1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Naᵥ1.8. Pain, 2002. 95(1-2): p. 143-52; Dong, X. W., et al., Small interfering RNA-mediated selective knockdown of Na(v)1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Navl.8) mRNA accumulation to sciatic nerve injury-induced painful neuropathy in rats. J. Biol. Chem. 286(46): p. 39836-47). The small DRG neurons where Naᵥ1.8 is expressed include the nociceptors involved in pain signaling. Naᵥ1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Naᵥ1.8 is necessary for rapid repetitive action potentials in nociceptors, and for spontaneous activity of damaged neurons (Choi, J. S. and S. G. Waxman, Physiological interactions between Naᵥ1.7 and Naᵥ1.8 sodium channels: a computer simulation study. J. Neurophysiol. 106(6): p. 3173-84; Renganathan, M., T. R. Cummins, and S. G. Waxman, Contribution of Na(v)1.8 sodium channels to action potential electrogenesis in DRG neurons. J. Neurophysiol., 2001. 86(2): p. 629-40; Roza, C., et al., The tetrodotoxin-resistant Na⁺ channel Naᵥ1.8 is essential for the expression of spontaneous activity in damaged sensory axons of mice. J. Physiol., 2003. 550(Pt 3): p. 921-6). In depolarized or damaged DRG neurons, Naᵥ1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Naᵥ1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Naᵥ1.7, Naᵥ1.8 and Naᵥ1.9 in a distinct population of dorsal root ganglia innervating the rat knee joint in a model of chronic inflammatory joint pain. Eur. J. Pain, 2008. 12(5): p. 564-72; Qiu, F., et al., Increased expression of tetrodotoxin-resistant sodium channels Naᵥ1.8 and Naᵥ1.9 within dorsal root ganglia in a rat model of bone cancer pain. Neurosci. Lett., 512(2): p. 61-6).

### SUMMARY

In one aspect, the disclosure relates to a method of treating chronic pain in a subject, comprising administering to the subject Compound 1:

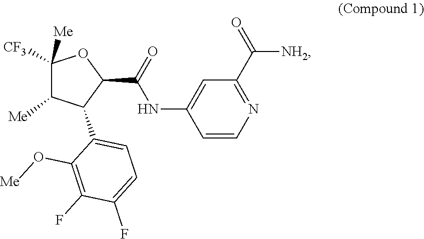 [SMILES: COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof, in an amount of 15-80 per day. In some aspects, about 70 mg per day of Compound 1 is administered to the subject. In some aspects, 70 mg per day of Compound 1 is administered to the subject.

In another aspect, the disclosure relates to a method of treating acute pain in a subject, comprising administering to the subject Compound 1, or a pharmaceutically acceptable salt thereof, in a first dose of 85-115 mg, followed by subsequent doses of 40-60 mg every 12 hours. In some aspects, the first dose is about 100 mg, followed by subsequent doses of about 50 mg every 12 hours. In some aspects, the first dose is 100 mg, followed by subsequent doses of 50 mg every 12 hours.

In another aspect, the disclosure relates to a pharmaceutical composition comprising: 45-55 wt % of a solid dispersion comprising a polymer and Compound 1; 40-50 wt % of one or more fillers; 2-4 wt % of a disintegrant; and 0.5-1.5 wt % of a lubricant.

In another aspect, the disclosure relates to a pharmaceutical composition comprising: 45-55 wt % of a solid dispersion comprising a polymer and Compound 1; 42-50 wt % of one or more fillers; 2-4 wt % of a disintegrant; and 0.5-1.5 wt % of a lubricant.

In another aspect, the disclosure relates to a process for preparing Compound 1, comprising transforming a compound of formula 5:

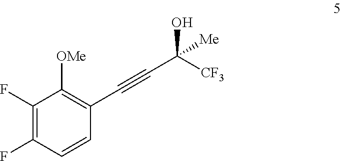 [SMILES: COc1c(C#C[C@@](C)(O)C(F)(F)F)ccc(F)c1F]

### to Compound 1.

### BRIEF DESCRIPTION OF THE DRAWINGS

FIG. 1 depicts an XRPD pattern of the spray-dried dispersion of Compound 1 of Example 2.

FIG. 2A depicts an XRPD pattern of the SDD tablet composition of Example 2 over the range of about 3° to about 40° 2θ.

FIG. 2B depicts an XRPD pattern of the SDD tablet composition of Example 2 over the range of about 14° to about 16° 2θ.

FIG. 3A depicts a solid state ¹⁹F NMR spectrum characteristic of the SDD tablet composition of Example 2.

FIG. 3B depicts a solid state ¹³C NMR spectrum characteristic of a powder of the SDD tablet composition of Example 2.

FIG. 4 depicts a graph of mean NPRS score versus time for each treatment group in the study described in Example 5.

FIG. 5 depicts a graph of mean NPRS score versus time for each treatment group in the study described in Example 6.

### DETAILED DESCRIPTION

### Definitions

The chemical elements are identified herein in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “amorphous” refers to a solid material having no long-range order in the position of its molecules. The molecules in an amorphous solid are generally arranged in a random manner with no well-defined arrangement. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern.

As used herein, the term “substantially amorphous” refers to a solid material having little or no long-range order in the position of its molecules. For example, substantially amorphous materials have less than about 15% crystallinity (e.g., less than about 10% crystallinity or less than about 5% crystallinity). The term ‘substantially amorphous’ includes materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. In other embodiments, a solid dispersion includes the drug constituting the dispersed phase, and the polymer constituting the continuous phase.

As used herein, the term “Compound 1,” and the structure and chemical name corresponding to the “Compound 1,” refer to a collection of molecules having identical chemical structures, namely the structure corresponding to the “Compound 1,” except that there may be isotopic variation among the constituent atoms of the molecules. The term “Compound 1” includes such a collection of molecules without regard to the purity of a given sample containing the collection of molecules. Thus, the term “Compound 1” includes such a collection of molecules in pure form or in a mixture (e.g., solution, suspension, or colloid) with one or more other substances.

In the specification and claims, unless otherwise specified, any atom not specifically designated as a particular isotope in Compound 1 is meant to represent any stable isotope of the specified element. In the Examples, where an atom is not specifically designated as a particular isotope, no effort was made to enrich that atom in a particular isotope, and therefore a person of ordinary skill in the art would understand that such atom likely was present at approximately the natural abundance isotopic composition of the specified element.

As used herein, the term “filler,” when referring to an excipient in a pharmaceutical composition, such as a tablet, refers to an excipient that increases the volume of the pharmaceutical composition. Examples of fillers include starches (e.g., microcrystalline cellulose), lactose, sucrose, glucose, mannitol, and silicic acid.

As used herein, the term “disintegrant,” when referring to an excipient in a pharmaceutical composition, such as a tablet, refers to an excipient that causes the pharmaceutical composition, to break down into smaller pieces when it comes into contact with a liquid, such as water. Examples of disintegrants include agar-agar, calcium carbonate, potato or tapioca starch, alginic acid, certain silicates, sodium carbonate, sodium starch glycolate, croscarmellose sodium, and crospovidone.

As used herein, the term “lubricant,” when referring to an excipient in a pharmaceutical composition, such as a tablet, refers to an excipient that facilitates the flow of a powder mixture and prevents the adhesion of the mixture to the surfaces of the die and punches during tablet compression. Examples of lubricants include calcium stearate, stearic acid, magnesium stearate, solid polyethylene glycols, sodium lauryl sulfate, and mixtures thereof.

As used herein, the term “glidant,” when referring to an excipient in a pharmaceutical composition, such as a tablet, refers to an excipient that enhances the flow of a granular or powder mixture by reducing interparticle friction, surface charge, and/or cohesion. Examples of glidants include tale and colloidal silicon dioxide.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein, “H” refers to hydrogen and includes any stable isotope of hydrogen, namely ¹H and D. In the Examples, where an atom is designated as “H,” no effort was made to enrich that atom in a particular isotope of hydrogen and, therefore, a person of ordinary skill in the art would understand that such hydrogen atom likely was present at approximately the natural abundance isotopic composition of hydrogen.

In some embodiments, Compound 1, or a pharmaceutically acceptable salt thereof, includes each constituent atom at approximately the natural abundance isotopic composition of the specified element.

In some embodiments, Compound 1, or a pharmaceutically acceptable salt thereof, includes one or more atoms having an atomic mass or mass number which differs from the atomic mass or mass number of the most abundant isotope of the specified element (“isotope-labelled” compound or salt). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The terms “Compound 1” and “pharmaceutically acceptable salt thereof” include the Compound 1 and any pharmaceutically acceptable salt thereof in any form, including any solid form thereof (including any amorphous or crystalline form thereof), any solvate, hydrate, or cocrystal form thereof, and any solution or suspension thereof.

As used herein, the term “about” includes the value of a specified amount or a range encompassing that specified amount that is recognized by one of ordinary skill in the art to provide a pharmacological effect equivalent to that obtained from the specified amount. The term “about” may refer to an acceptable error for a particular value as determined by one of skill in the art, which depends in part on how the value is measured or determined. In some embodiments, the term “about” means within 20%, 15%, 10%, 5%, 4%, 3%, 2%, 1% or 0.5% of a given value or range. In some embodiments, the term “about” means within 20% of a given value or range. In some embodiments, the term “about” means within 15% of a given value or range. In some embodiments, the term “about” means within 10% of a given value or range. In some embodiments, the term “about” means within 5% of a given value or range. In some embodiments, the term “about” means within 1% of a given value or range. In some embodiments, the term “about” means within 0.5% of a given value or range.

As used herein in connection with a medical condition (e.g., chronic pain or acute pain), the term “treating” includes both providing full relief from the condition (e.g., providing full pain relief) and providing partial relief from the condition (e.g., providing partial pain relief or lessening the severity of the pain).

As used herein, the term “subject” or “patient” means an animal, preferably a mammal, and most preferably a human.

As used herein, the term “amount,” when referring to an amount of Compound 1, or a pharmaceutically acceptable salt thereof, administered to a subject, refers to the mass of an equimolar amount of (2R,3S,4S,5R)-4-[[3-(3,4-difluoro-2-methoxy-phenyl)-4,5-dimethyl-5-(trifluoromethyl) tetrahydrofuran-2-carbonyl]amino]pyridine-2-carboxamide, regardless of the actual mass of any salt, solvate, hydrate, or cocrystal form that may be administered.

In certain embodiments, an “effective amount” of Compound 1, a pharmaceutically acceptable salt thereof, or a pharmaceutical composition thereof is that amount effective for treating one or more of the conditions recited herein.

As used herein, the term “course of treatment,” when referring to Compound 1, or a pharmaceutically acceptable salt thereof, refers to the administration of one or more doses of the compound or salt during a period of time that is separate from any earlier or later administration of the compound or salt. Typically, Compound 1 and any metabolites thereof are substantially eliminated from a subject's systemic circulation between courses of treatment.

As used herein, the term “dose,” when referring to the administration of Compound 1, or a pharmaceutically acceptable salt thereof, refers to an amount of the compound or salt administered in a discrete period of time, separate from other amounts of the compound or salt that may be administered at other times during the same day or course of treatment. When a dose is administered orally, the dose may be administered in a single tablet, capsule, or other oral dosage form, or in multiple such dosage forms.

As used herein, the term “first dose” refers to the first dose of Compound 1, or a pharmaceutically acceptable salt thereof, that is administered in a given course of treatment.

As used herein, the term “subsequent dose” refers to any dose of Compound 1, or a pharmaceutically acceptable salt thereof, that is administered after the first dose in a given course of treatment.

As used herein, the term “baseline pain score” refers to a subject's pain score, such as a score on the 11-point Numeric Pain Rating Scale or Verbal Categorical Rating Scale, prior to beginning a course of treatment with Compound 1, or a pharmaceutically acceptable salt thereof.

As used herein, the term “11-point Numeric Pain Rating Scale” (also known as “NPRS”) refers to a pain rating scale on which a subject rates his or her pain intensity on a scale of 0 to 10, where a score of 0 denotes no pain, and a score of 10 denotes the worst pain intensity imaginable.

As used herein, the term “Verbal Categorical Rating Scale” refers to a pain rating scale on which a subject rates his or her pain intensity as none, mild, moderate, or severe.

As used herein, the term “adverse event” is defined as any untoward medical occurrence in a subject during a study; the event does not necessarily have a causal relationship with the treatment.

This includes any newly occurring event or worsening of a pre-existing condition (e.g., increase in its severity or frequency).

As used herein, an abnormal study assessment is considered “clinically significant” if the subject has 1 or more of the following: concomitant signs or symptoms related to the abnormal study assessment, further diagnostic testing or medical/surgical intervention, a change in the dose of study drug or discontinuation from the study. The determination of whether the study assessment results are clinically significant will be made by the investigator.

### Medical Uses of Compound 1 or a Pharmaceutically Acceptable Salt Thereof

### Methods of Treatment

In yet another aspect, the disclosure relates to a method of treating pain in a subject, comprising administering an effective amount of Compound 1, a pharmaceutically acceptable salt thereof or a pharmaceutical composition thereof.

In yet another aspect, the disclosure relates to a method of treating chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia in a subject, comprising administering an effective amount of Compound 1, a pharmaceutically acceptable salt thereof or a pharmaceutical composition thereof.

In yet another aspect, the disclosure relates to a method of treating chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia in a subject, comprising administering an effective amount of Compound 1, a pharmaceutically acceptable salt thereof or a pharmaceutical composition thereof.

In yet another aspect, the disclosure relates to a method of treating gut pain in a subject, wherein gut pain comprises inflammatory bowel disease pain, Crohn's disease pain, irritable bowel syndrome, endometriosis, polycystic ovarian disease, salpingitis, cervicitis or interstitial cystitis pain wherein said method comprises administering an effective amount of Compound 1, a pharmaceutically acceptable salt thereof or a pharmaceutical composition thereof.

In yet another aspect, the disclosure relates to a method of treating neuropathic pain in a subject comprising administering an effective amount of Compound 1, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the disclosure relates to a method of treating neuropathic pain in a subject, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of Compound 1, a pharmaceutically acceptable salt thereof or a pharmaceutical composition thereof.

In yet another aspect, the disclosure relates to a method of treating musculoskeletal pain in a subject, comprising administering an effective amount of Compound 1, a pharmaceutically acceptable salt thereof or a pharmaceutical composition thereof. In some aspects, the musculoskeletal pain comprises osteoarthritis pain.

In yet another aspect, the disclosure relates to a method of treating musculoskeletal pain in a subject, wherein musculoskeletal pain comprises osteoarthritis pain, back pain, cold pain, burn pain or dental pain wherein said method comprises administering an effective amount of Compound 1, a pharmaceutically acceptable salt thereof or a pharmaceutical composition thereof.

In yet another aspect, the disclosure relates to a method of treating inflammatory pain in a subject, wherein inflammatory pain comprises rheumatoid arthritis pain, ankylosing spondylitis or vulvodynia wherein said method comprises administering an effective amount of Compound 1, a pharmaceutically acceptable salt thereof or a pharmaceutical composition thereof.

In yet another aspect, the disclosure relates to a method of treating inflammatory pain in a subject, wherein inflammatory pain comprises rheumatoid arthritis pain wherein said method comprises administering an effective amount of Compound 1, a pharmaceutically acceptable salt thereof or a pharmaceutical composition thereof.

In yet another aspect, the disclosure relates to a method of treating idiopathic pain in a subject, wherein idiopathic pain comprises fibromyalgia pain wherein said method comprises administering an effective amount of Compound 1, a pharmaceutically acceptable salt thereof or a pharmaceutical composition thereof.

In yet another aspect, the disclosure relates to a method of treating idiopathic pain in a subject, wherein idiopathic pain comprises reflex sympathetic dystrophy pain, wherein said method comprises administering an effective amount of Compound 1, a pharmaceutically acceptable salt thereof or a pharmaceutical composition thereof.

In yet another aspect, the disclosure relates to a method of treating pathological cough in a subject, wherein said method comprises administering an effective amount of Compound 1, a pharmaceutically acceptable salt thereof or a pharmaceutical composition thereof.

In yet another aspect, the disclosure relates to a method of treating acute pain in a subject, comprising administering an effective amount of Compound 1, a pharmaceutically acceptable salt thereof or a pharmaceutical composition thereof. In some aspects, the acute pain comprises acute post-operative pain.

In yet another aspect, the disclosure relates to a method of treating postsurgical pain in a subject (e.g., joint replacement pain, soft tissue surgery pain, post-thoracotomy pain, post-mastectomy pain, hemorrhoidectomy pain, herniorrhaphy pain, bunionectomy pain or abdominoplasty pain), comprising administering an effective amount of Compound 1, a pharmaceutically acceptable salt thereof or a pharmaceutical composition thereof.

In yet another aspect, the disclosure relates to a method of treating bunionectomy pain in a subject, comprising administering an effective amount of Compound 1, a pharmaceutically acceptable salt thereof or a pharmaceutical composition thereof.

In yet another aspect, the disclosure relates to a method of treating shoulder arthroplasty pain or shoulder arthroscopy pain in a subject, comprising administering an effective amount of Compound 1, a pharmaceutically acceptable salt thereof or a pharmaceutical composition thereof.

In yet another aspect, the disclosure relates to a method of treating herniorrhaphy pain in a subject, comprising administering an effective amount of Compound 1, a pharmaceutically acceptable salt thereof or a pharmaceutical composition thereof.

In yet another aspect, the disclosure relates to a method of treating abdominoplasty pain in a subject, comprising administering an effective amount of Compound 1, a pharmaceutically acceptable salt thereof or a pharmaceutical composition thereof.

In yet another aspect, the disclosure relates to a method of treating visceral pain in a subject, comprising administering an effective amount of Compound 1, a pharmaceutically acceptable salt thereof or a pharmaceutical composition thereof. In some aspects, the visceral pain comprises visceral pain from abdominoplasty.

In yet another aspect, the disclosure relates to a method of treating a neurodegenerative disease in a subject, comprising administering an effective amount of Compound 1, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the disclosure relates to a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the disclosure relates to a method of inhibiting a voltage-gated sodium channel in a biological sample comprising contacting the biological sample with an effective amount of Compound 1, a pharmaceutically acceptable salt thereof or a pharmaceutical composition thereof. In another aspect, the voltage-gated sodium channel is Naᵥ1.8.

In another aspect, the disclosure relates to a method of treating acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility in a subject, comprising administering an effective amount of Compound 1, a pharmaceutically acceptable salt thereof or a pharmaceutical composition thereof.

In another aspect, the disclosure relates to a method of treating femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain in a subject, comprising administering an effective amount of Compound 1, a pharmaceutically acceptable salt thereof or a pharmaceutical composition thereof.

In another aspect, the disclosure relates to a method of treating trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain in a subject, comprising administering an effective amount of Compound 1, a pharmaceutically acceptable salt thereof or a pharmaceutical composition thereof.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale). In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain). In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale). In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences about a 51% reduction (e.g., a 51% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences about a 47% reduction (e.g., a 47% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline.

In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences about a 51% reduction (e.g., a 51% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the first dose is about 100 mg, and the subsequent doses are about 50 mg every 12 hours. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline.

In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences about a 47% reduction (e.g., a 47% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the disclosure relates to a method of treating acute pain in a subject, comprising administering Compound 1, or a pharmaceutically acceptable salt thereof, in a first dose of 100 mg, followed by subsequent doses of 50 mg every 12 hours, wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale).

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences about a 51% reduction (e.g., a 51% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences about a 47% reduction (e.g., a 47% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences about a 51% reduction (e.g., a 51% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences about a 47% reduction (e.g., a 47% reduction) in pain intensity on an 11-point Numeric Pain Rating Scale (NPRS) after 48 hours, relative to baseline. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein the acute pain is acute post-operative pain or postsurgical pain (e.g., bunionectomy pain, abdominoplasty pain, herniorrhaphy pain), wherein the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale), wherein the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating or lessening the severity of acute pain in a subject, comprising administering Compound 1, or a pharmaceutically acceptable salt thereof, in a first dose of 85-115 mg, followed by subsequent doses of 40-60 mg every 12 hours. In some embodiments, the acute pain is acute post-operative pain or postsurgical pain (e.g., bunionectomy pain, abdominoplasty pain, herniorrhaphy pain). In some embodiments, the acute pain is moderate to severe acute pain (e.g., wherein the subject has a baseline pain score of ≥4 on an 11-point Numeric Pain Rating Scale (NPRS) or where the subject has a baseline pain level of moderate or severe on a Verbal Categorical Rating Scale). In some embodiments, the subject experiences a time-weighted sum of pain intensity difference, relative to baseline, as recorded on an 11-point Numeric Pain Rating Scale (NPRS), from 0 to 48 hours (SPID48) of 75.0 to 125.0 (e.g., 95.0 to 105.0, or about 99.9). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0 (e.g., 25.0 to 35.0, or about 29.3). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 51% reduction (e.g., a 51% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 115 to 480 minutes (e.g., 210 to 270 minutes, or about 240 minutes) after administration of the first dose. In some embodiments, the subject experiences a time-weighted sum of pain intensity difference, relative to baseline, as recorded on an 11-point Numeric Pain Rating Scale (NPRS), from 0 to 48 hours (SPID48) of 100.0 to 140.0 (e.g., 113.0 to 123.0, or about 118.4). In some embodiments, the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0 (e.g., 43.0 to 53.0, or about 48.4). In some embodiments, the subject experiences a change of about −2.5 to about −4.5 (e.g., −2.5 to −4.5, or about −3.0 to about −4.0, or −3.0 to −4.0, or about −3.4, or −3.4) in pain intensity on an 11-point Numeric Pain Rating Scale (NPRS) after 48 hours, relative to baseline. In some embodiments, the subject experiences about a 47% reduction (e.g., a 47% reduction) in pain intensity on an 11-point Numeric Pain Rating Scale (NPRS) after 48 hours, relative to baseline. In some embodiments, the subject experiences a 2-point reduction in pain intensity, relative to baseline, as recorded on an 11-point Numeric Pain Rating Scale (NPRS), 90 to 180 minutes (e.g., 100 to 140 minutes, or about 119 minutes) after administration of the first dose. In some embodiments, the acute pain is fully relieved in the subject. In some embodiments, the acute pain is lessened in the subject. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating acute pain in a subject, comprising administering Compound 1, or a pharmaceutically acceptable salt thereof, in a first dose of 85-115 mg, followed by subsequent doses of 40-60 mg every 12 hours, wherein Compound 1 is administered in a pharmaceutical composition comprising: 45-55 wt % of a solid dispersion comprising a polymer and Compound 1; 42-50 wt % of one or more fillers; 2-4 wt % of a disintegrant; and 0.5-1.5 wt % of a lubricant. In some embodiments, the one or more fillers comprise microcrystalline cellulose. In some embodiments, the disintegrant is croscarmellose sodium. In some embodiments, the lubricant is magnesium stearate. In some embodiments, the pharmaceutical composition comprises: 45-55 wt % of a solid dispersion comprising a polymer and Compound 1; 42-50 wt % of microcrystalline cellulose; 2-4 wt % of croscarmellose sodium; and 0.5-1.5 wt % of magnesium stearate. In some embodiments, the pharmaceutical composition is a tablet core composition. In some embodiments, the tablet core composition is coated with a tablet coating (e.g., Opadry Blue). In some embodiments, the pharmaceutical composition comprises about 50 mg of Compound 1. In some embodiments, the polymer is HPMCAS. In some embodiments, the solid dispersion comprises 70-80 wt % of the polymer and 20-30 wt % of Compound 1 (e.g., about 75 wt % of the polymer and about 25 wt % of Compound 1). In some embodiments, the pharmaceutical composition comprises about 50 wt % of the solid dispersion; about 46 wt % of microcrystalline cellulose; about 3 wt % of croscarmellose sodium; and about 1.0 wt % of magnesium stearate. In some embodiments, the pharmaceutical composition comprises about 50 wt % of the solid dispersion, wherein the solid dispersion comprises about 75 wt % of the polymer (e.g., HPMCAS) and about 25 wt % of Compound 1; about 46 wt % of microcrystalline cellulose; about 3 wt % of croscarmellose sodium; and about 1.0 wt % of magnesium stearate. In some embodiments, the pharmaceutical composition comprises 50 wt % of the solid dispersion, wherein the solid dispersion comprises about 75 wt % of the polymer (e.g., HPMCAS) and about 25 wt % of Compound 1; 46 wt % of microcrystalline cellulose; 3 wt % of croscarmellose sodium; and 1.0 wt % of magnesium stearate. In some embodiments, the pharmaceutical composition comprises 50 wt % of the solid dispersion, wherein the solid dispersion comprises 75 wt % of the polymer (e.g., HPMCAS) and 25 wt % of Compound 1; 46 wt % of microcrystalline cellulose; 3 wt % of croscarmellose sodium; and 1.0 wt % of magnesium stearate. In some embodiments, the pharmaceutical composition comprises about 184 mg of microcrystalline cellulose. In some embodiments, the pharmaceutical composition comprises about 12 mg of croscarmellose sodium. In some embodiments, the pharmaceutical composition comprises about 4 mg of magnesium stearate. In some embodiments, the pharmaceutical composition comprises about 200 mg of a solid dispersion comprising a polymer and Compound 1. In some embodiments, the pharmaceutical composition comprises about 200 mg of a solid dispersion comprising a polymer (e.g., HPMCAS) and Compound 1, wherein the solid dispersion comprises about 150 mg of the polymer (e.g., HPMCAS) and about 50 mg of Compound 1; about 184 mg of microcrystalline cellulose; about 12 mg of croscarmellose sodium; and about 4 mg of magnesium stearate. In some embodiments, the pharmaceutical composition comprises 200 mg of a solid dispersion comprising a polymer (e.g., HPMCAS) and Compound 1, wherein the solid dispersion comprises 150 mg of the polymer (e.g., HPMCAS) and 50 mg of Compound 1; 184 mg of microcrystalline cellulose; 12 mg of croscarmellose sodium; and 4 mg of magnesium stearate. In some embodiments, the pharmaceutical composition comprises an intragranular blend and extragranular blend. In some embodiments, the intragranular blend comprises 45-55 wt % of a solid dispersion comprising a polymer and Compound 1; 20-25 wt % of a filler; 1-2 wt % of a disintegrant; and 0-0.2 wt % of a lubricant; and the extragranular blend comprises 20-25 wt % of a filler; 1-2 wt % of a disintegrant; and 0.8-1.0 wt % of a lubricant, wherein such weight percentages are based on the combined weights of the intragranular and extragranular blends. In some embodiments, the intragranular blend comprises 45-55 wt % of a solid dispersion comprising a polymer and Compound 1; 20-25 wt % of microcrystalline cellulose; 1-2 wt % of croscarmellose sodium; and 0-0.2 wt % of magnesium stearate; and the extragranular blend comprises 20-25 wt % of microcrystalline cellulose; 1-2 wt % of croscarmellose sodium; and 0.8-1.0 wt % of magnesium stearate, wherein such weight percentages are based on the combined weights of the intragranular and extragranular blends. In some embodiments, the intragranular blend comprises about 50 wt % of a solid dispersion comprising a polymer and Compound 1, wherein the solid dispersion comprises about 75 wt % of the polymer (e.g., HPMCAS) and about 25 wt % of Compound 1; about 23.4 wt % of microcrystalline cellulose; about 1.5 wt % of croscarmellose sodium; and about 0.1 wt % of magnesium stearate; and the extragranular blend comprises about 22.6 wt % of microcrystalline cellulose; about 1.5 wt % of croscarmellose sodium; and about 0.9 wt % of magnesium stearate, wherein such weight percentages are based on the combined weights of the intragranular and extragranular blends. In some embodiments, the intragranular blend comprises 50 wt % of a solid dispersion comprising a polymer and Compound 1, wherein the solid dispersion comprises 75 wt % of the polymer (e.g., HPMCAS) and 25 wt % of Compound 1; 23.4 wt % of microcrystalline cellulose; 1.5 wt % of croscarmellose sodium; and 0.1 wt % of magnesium stearate; and the extragranular blend comprises 22.6 wt % of microcrystalline cellulose; 1.5 wt % of croscarmellose sodium; and 0.9 wt % of magnesium stearate, wherein such weight percentages are based on the combined weights of the intragranular and extragranular blends. In some embodiments, the intragranular blend comprises about 200 mg of the solid dispersion, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate. In some embodiments, the intragranular blend comprises about 200 mg of the solid dispersion, wherein the solid dispersion comprises about 150 mg of the polymer (e.g., HPMCAS) and about 50 mg of Compound 1, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate. In some embodiments, the intragranular blend comprises 200 mg of the solid dispersion, wherein the solid dispersion comprises 150 mg of the polymer (e.g., HPMCAS) and 50 mg of Compound 1, 93.6 mg of microcrystalline cellulose, 6.0 mg of croscarmellose sodium, and 0.4 mg of magnesium stearate; and the extragranular blend comprises 90.4 mg of microcrystalline cellulose, 6.0 mg of croscarmellose sodium, and 3.6 mg of magnesium stearate. In some embodiments, the intragranular blend comprises about 0.4 mg of magnesium stearate, and the extragranular blend comprises about 3.6 mg of magnesium stearate. In some embodiments, the intragranular blend comprises 0.4 mg of magnesium stearate, and the extragranular blend comprises 3.6 mg of magnesium stearate. In some embodiments, the first dose is about 85 to about 115 mg, and the subsequent doses are about 40 to about 60 mg every 12 hours. In some embodiments, the first dose is about 100 mg, and the subsequent doses are about 50 mg every 12 hours. In some embodiments, the first dose is 100 mg, and the subsequent doses are 50 mg every 12 hours.

In another aspect, the disclosure relates to a method of treating chronic pain in a subject, comprising administering Compound 1, or a pharmaceutically acceptable salt thereof, in an amount of 15-80 mg per day. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In another aspect, the disclosure relates to a method of treating chronic pain in a subject, comprising administering Compound 1, or a pharmaceutically acceptable salt thereof, in an amount of about 46 mg per day. In another aspect, the disclosure relates to a method of treating chronic pain in a subject, comprising administering Compound 1, or a pharmaceutically acceptable salt thereof, in an amount of 45 mg per day. In another aspect, the disclosure relates to a method of treating chronic pain in a subject, comprising administering Compound 1, or a pharmaceutically acceptable salt thereof, in an amount of about 69 mg per day. In another aspect, the disclosure relates to a method of treating chronic pain in a subject, comprising administering Compound 1, or a pharmaceutically acceptable salt thereof, in an amount of 70 mg per day.

In another aspect, the disclosure relates to a method of treating chronic pain in a subject, comprising administering Compound 1, or a pharmaceutically acceptable salt thereof, in an amount of 15-80 mg per day, wherein the chronic pain is moderate to severe chronic pain (e.g., wherein the subject has a baseline weekly average pain score of ≥4 on an 11-point Numeric Pain Rating Scale (NPRS)). In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In another aspect, the disclosure relates to a method of treating chronic pain in a subject, comprising administering Compound 1, or a pharmaceutically acceptable salt thereof, in an amount of about 46 mg per day, wherein the chronic pain is moderate to severe chronic pain. In another aspect, the disclosure relates to a method of treating chronic pain in a subject, comprising administering Compound 1, or a pharmaceutically acceptable salt thereof, in an amount of 45 mg per day, wherein the chronic pain is moderate to severe chronic pain. In another aspect, the disclosure relates to a method of treating chronic pain in a subject, comprising administering Compound 1, or a pharmaceutically acceptable salt thereof, in an amount of about 69 mg per day, wherein the chronic pain is moderate to severe chronic pain. In another aspect, the disclosure relates to a method of treating chronic pain in a subject, comprising administering Compound 1, or a pharmaceutically acceptable salt thereof, in an amount of 70 mg per day, wherein the chronic pain is moderate to severe chronic pain.

In another aspect, the disclosure relates to a method of treating chronic pain in a subject, comprising administering Compound 1, or a pharmaceutically acceptable salt thereof, in an amount of 15-80 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy, pain caused by radiculopathy (e.g., lumbosacral radiculopathy)), musculoskeletal pain (e.g., osteoarthritis pain), or visceral pain. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy, pain caused by radiculopathy (e.g., lumbosacral radiculopathy)), musculoskeletal pain (e.g., osteoarthritis pain), or visceral pain. In some embodiments, the disclosure relates to a method of treating chronic pain in a subject, comprising administering Compound 1, or a pharmaceutically acceptable salt thereof, in an amount of about 45 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy, pain caused by radiculopathy (e.g., lumbosacral radiculopathy)), musculoskeletal pain (e.g., osteoarthritis pain), or visceral pain. In some embodiments, the disclosure relates to a method of treating chronic pain in a subject, comprising administering Compound 1, or a pharmaceutically acceptable salt thereof, in an amount of about 69 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy, pain caused by radiculopathy (e.g., lumbosacral radiculopathy)), musculoskeletal pain (e.g., osteoarthritis pain), or visceral pain. In some embodiments, the disclosure relates to a method of treating chronic pain in a subject, comprising administering Compound 1, or a pharmaceutically acceptable salt thereof, in an amount of about 70 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy, pain caused by radiculopathy (e.g., lumbosacral radiculopathy)), musculoskeletal pain (e.g., osteoarthritis pain), or visceral pain.

In another aspect, the disclosure relates to a method of treating chronic pain in a subject, comprising administering Compound 1, or a pharmaceutically acceptable salt thereof, in an amount of 15-80 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)). In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 45 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 69 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 70 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)).

In another aspect, the disclosure relates to a method of treating chronic pain in a subject, comprising administering Compound 1, or a pharmaceutically acceptable salt thereof, in an amount of 15-80 mg per day, wherein the chronic pain is diabetic peripheral neuropathy. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is diabetic peripheral neuropathy. In some embodiments, the disclosure relates to a method of treating chronic pain in a subject, comprising administering Compound 1, or a pharmaceutically acceptable salt thereof, in an amount of about 45 mg per day, wherein the chronic pain is diabetic peripheral neuropathy. In some embodiments, the disclosure relates to a method of treating chronic pain in a subject, comprising administering Compound 1, or a pharmaceutically acceptable salt thereof, in an amount of about 69 mg per day, wherein the chronic pain is diabetic peripheral neuropathy. In some embodiments, the disclosure relates to a method of treating chronic pain in a subject, comprising administering Compound 1, or a pharmaceutically acceptable salt thereof, in an amount of about 70 mg per day, wherein the chronic pain is diabetic peripheral neuropathy.

In another aspect, the disclosure relates to a method of treating chronic pain in a subject, comprising administering Compound 1, or a pharmaceutically acceptable salt thereof, in an amount of 15-80 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)). In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is diabetic peripheral neuropathy, wherein the chronic pain is moderate to severe chronic pain (e.g., wherein the subject has a baseline weekly average pain score of 4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 45 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 69 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 70 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)).

In another aspect, the disclosure relates to a method of treating chronic pain in a subject, comprising administering Compound 1, or a pharmaceutically acceptable salt thereof, in an amount of 15-80 mg per day, wherein the chronic pain is lumbosacral radiculopathy. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is lumbosacral radiculopathy. In some embodiments, the disclosure relates to a method of treating chronic pain in a subject, comprising administering Compound 1, or a pharmaceutically acceptable salt thereof, in an amount of about 45 mg per day, wherein the chronic pain is lumbosacral radiculopathy. In some embodiments, the disclosure relates to a method of treating chronic pain in a subject, comprising administering Compound 1, or a pharmaceutically acceptable salt thereof, in an amount of about 69 mg per day, wherein the chronic pain is lumbosacral radiculopathy. In some embodiments, the disclosure relates to a method of treating chronic pain in a subject, comprising administering Compound 1, or a pharmaceutically acceptable salt thereof, in an amount of about 70 mg per day, wherein the chronic pain is lumbosacral radiculopathy.

In another aspect, the disclosure relates to a method of treating chronic pain in a subject, comprising administering Compound 1, or a pharmaceutically acceptable salt thereof, in an amount of 15-80 mg per day, wherein the chronic pain is lumbosacral radiculopathy, wherein the chronic pain is moderate to severe chronic pain (e.g., wherein the subject has a baseline weekly average pain score of ≥4, or ≥4 and <10, or ≥4 and <9, on an 11-point Numeric Pain Rating Scale (NPRS)). In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is lumbosacral radiculopathy, wherein the chronic pain is moderate to severe chronic pain (e.g., wherein the subject has a baseline weekly average pain score of ≥4, or ≥4 and ≤10, or ≥4 and ≤9, on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 45 mg per day, wherein the chronic pain is lumbosacral radiculopathy, wherein the chronic pain is moderate to severe chronic pain (e.g., wherein the subject has a baseline weekly average pain score of ≥4, or ≥4 and ≤10, or ≥4 and ≤9, on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 69 mg per day, wherein the chronic pain is lumbosacral radiculopathy, wherein the chronic pain is moderate to severe chronic pain (e.g., wherein the subject has a baseline weekly average pain score of ≥4, or ≥4 and ≤10, or ≥4 and ≤9, on an 11-point Numeric Pain Rating Scale (NPRS)). In some embodiments, the disclosure relates to a method of treating chronic pain in a subject, comprising administering Compound 1, or a pharmaceutically acceptable salt thereof, in an amount of about 70 mg per day, wherein the chronic pain is lumbosacral radiculopathy, wherein the chronic pain is moderate to severe chronic pain (e.g., wherein the subject has a baseline weekly average pain score of ≥4, or ≥4 and ≤10, or ≥4 and ≤9, on an 11-point Numeric Pain Rating Scale (NPRS)).

In another aspect, the disclosure relates to a method of treating chronic pain in a subject, comprising administering Compound 1, or a pharmaceutically acceptable salt thereof, in an amount of 15-80 mg per day, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 45 mg per day, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 70 mg per day, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is moderate to severe chronic pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of 45 mg per day, wherein the chronic pain is moderate to severe chronic pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the chronic pain is moderate to severe chronic pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of 70 mg per day, wherein the chronic pain is moderate to severe chronic pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 45 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 70 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 45 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 70 mg per day, wherein the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein the chronic pain is diabetic peripheral neuropathy, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is diabetic peripheral neuropathy, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 45 mg per day, wherein the chronic pain is diabetic peripheral neuropathy, wherein the subject experiences a change of −1 to −4 (e.g., −2.0 to −2.5, or −2.18, or −2.11, or −2.26) in weekly average of daily pain on an 1I-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the chronic pain is diabetic peripheral neuropathy, wherein the subject experiences a change of −1 to −4 (e.g., −2.0 to −2.5, or −2.18 or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 70 mg per day, wherein the chronic pain is diabetic peripheral neuropathy, wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the disclosure relates to a method of treating chronic pain in a subject, comprising administering Compound 1, or a pharmaceutically acceptable salt thereof, in an amount of about 46 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 45 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 69 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the disclosure relates to a method of treating chronic pain in a subject, comprising administering Compound 1, or a pharmaceutically acceptable salt thereof, in an amount of about 70 mg per day, wherein the chronic pain is diabetic peripheral neuropathy, wherein the chronic pain is moderate to severe chronic pain (e.g., wherein the subject has a baseline weekly average pain score of ≥4 on an 11-point Numeric Pain Rating Scale (NPRS)), wherein the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating or lessening the severity of chronic pain in a subject, comprising administering Compound 1, or a pharmaceutically acceptable salt thereof, in an amount of 15-80 mg per day. In some embodiments, the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain. In some embodiments, the chronic pain is moderate to severe chronic pain (e.g., wherein the subject has a baseline weekly average pain score of ≥4 on an 11-point Numeric Pain Rating Scale (NPRS)). In some embodiments, Compound 1, or a pharmaceutically acceptable salt thereof, is administered in an amount of about 15 to about 80 mg per day, or about 20 to about 75 mg per day, or about 20 to about 30 mg per day, or about 40 about 50 mg per day, or about 60 to about 70 mg per day. In some embodiments, Compound 1, or a pharmaceutically acceptable salt thereof, is administered in an amount of 20-75 mg per day, or 20-30 mg per day, or 40-50 mg per day, or 60-70 mg per day. In some embodiments, Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day. In some embodiments, the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In some embodiments, the disclosure relates to a method of treating or lessening the severity of chronic pain in a subject, comprising administering Compound 1, or a pharmaceutically acceptable salt thereof, in an amount of about 46 mg per day. In some embodiments, the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain. In some embodiments, the chronic pain is moderate to severe chronic pain (e.g., wherein the subject has a baseline weekly average pain score of ≥4 on an 11-point Numeric Pain Rating Scale (NPRS)). In some embodiments, the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In some embodiments, the disclosure relates to a method of treating or lessening the severity of chronic pain in a subject, comprising administering Compound 1, or a pharmaceutically acceptable salt thereof, in an amount of about 45 mg per day. In some embodiments, the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain. In some embodiments, the chronic pain is moderate to severe chronic pain (e.g., wherein the subject has a baseline weekly average pain score of ≥4 on an 11-point Numeric Pain Rating Scale (NPRS)). In some embodiments, the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In some embodiments, the disclosure relates to a method of treating or lessening the severity of chronic pain in a subject, comprising administering Compound 1, or a pharmaceutically acceptable salt thereof, in an amount of about 69 mg per day. In some embodiments, the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain. In some embodiments, the chronic pain is moderate to severe chronic pain (e.g., wherein the subject has a baseline weekly average pain score of ≥4 on an 11-point Numeric Pain Rating Scale (NPRS)). In some embodiments, the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In some embodiments, the disclosure relates to a method of treating or lessening the severity of chronic pain in a subject, comprising administering Compound 1, or a pharmaceutically acceptable salt thereof, in an amount of about 70 mg per day. In some embodiments, the chronic pain is neuropathic pain (e.g., post-herpetic neuralgia, small-fiber neuropathy, idiopathic small-fiber neuropathy, diabetic neuropathy, diabetic peripheral neuropathy), pain caused by radiculopathy (e.g., lumbosacral radiculopathy)), musculoskeletal pain (e.g., osteoarthritis pain), or visceral pain. In some embodiments, the chronic pain is moderate to severe chronic pain (e.g., wherein the subject has a baseline weekly average pain score of ≥4 on an 11-point Numeric Pain Rating Scale (NPRS)). In some embodiments, the subject experiences a change of −1 to −4 (e.g., −2.0 to −2.5, or −2.18, or −2.11, or −2.26) in weekly average of daily pain on an 11-point Numeric Pain Rating Scale (NPRS) at week 12 of treatment, relative to baseline. In some embodiments, the chronic pain is fully relieved in the subject. In some embodiments, the chronic pain is lessened in the subject.

In another aspect, the disclosure relates to a method of treating chronic pain in a subject, comprising administering Compound 1, or a pharmaceutically acceptable salt thereof, in an amount of 15-80 mg per day, wherein Compound 1 is administered in a pharmaceutical composition comprising: 45-55 wt % of a solid dispersion comprising a polymer and Compound 1; 40-50 wt % of one or more fillers; 2-4 wt % of a disintegrant; 0.5-1.5 wt % of a lubricant; and 0.05-0.8 wt % (e.g., 0.1-0.3 wt %) of a glidant. In some embodiments, the pharmaceutical composition comprises: 45-55 wt % of a solid dispersion comprising a polymer and Compound 1; 40-50 wt % of one or more fillers; 2-4 wt % of a disintegrant; and 0.5-1.5 wt % of a lubricant. In some embodiments, the one or more fillers comprise microcrystalline cellulose and mannitol. In some embodiments, the disintegrant is croscarmellose sodium. In some embodiments, the lubricant is magnesium stearate. In some embodiments, the glidant is colloidal silicon dioxide. In some embodiments, the pharmaceutical composition comprises: 45-55 wt % of a solid dispersion comprising a polymer and Compound 1; 20-25 wt % of microcrystalline cellulose; 20-25 wt % of mannitol; 2-4 wt % of croscarmellose sodium; 0.5-1.5 wt % of magnesium stearate; and 0.1-0.3 wt % of colloidal silicon dioxide. In some embodiments, the pharmaceutical composition comprises: 45-55 wt % of a solid dispersion comprising a polymer and Compound 1; 20-25 wt % of microcrystalline cellulose; 20-25 wt % of mannitol; 2-4 wt % of croscarmellose sodium; and 0.5-1.5 wt % of magnesium stearate. In some embodiments, the pharmaceutical composition is a tablet core composition. In some embodiments, the tablet core composition is coated with a tablet coating (e.g., Opadry Blue). In some embodiments, the pharmaceutical composition comprises about 70 mg of Compound 1. In some embodiments, the polymer is HPMCAS. In some embodiments, the solid dispersion comprises 70-80 wt % of the polymer and 20-30 wt % of Compound 1 (e.g., about 75 wt % of the polymer and about 25 wt % of Compound 1). In some embodiments, the pharmaceutical composition comprises about 50 wt % of the solid dispersion; about 22.9 wt % of microcrystalline cellulose; about 22.9 wt % of mannitol; about 3 wt % of croscarmellose sodium; about 1.0 wt % of magnesium stearate; and about 0.2 wt % of colloidal silicon dioxide. In some embodiments, the pharmaceutical composition comprises about 50 wt % of the solid dispersion; about 22.9 wt % of microcrystalline cellulose; about 22.9 wt % of mannitol; about 3 wt % of croscarmellose sodium; and about 1.0 wt % of magnesium stearate. In some embodiments, the pharmaceutical composition comprises about 50 wt % of a solid dispersion comprising a polymer (e.g., HPMCAS) and Compound 1, wherein the solid dispersion comprises about 75 wt % of the polymer (e.g., HPMCAS) and about 25 wt % of Compound 1; about 22.9 wt % of microcrystalline cellulose; about 22.9 wt % of mannitol; about 3 wt % of croscarmellose sodium; about 1.0 wt % of magnesium stearate; and about 0.2 wt % of colloidal silicon dioxide. In some embodiments, the pharmaceutical composition comprises 50 wt % of of a solid dispersion comprising a polymer (e.g., HPMCAS) and Compound 1, wherein the solid dispersion comprises 75 wt % of the polymer (e.g., HPMCAS) and 25 wt % of Compound 1; 22.9 wt % of microcrystalline cellulose; 22.9 wt % of mannitol; 3 wt % of croscarmellose sodium; 1.0 wt % of magnesium stearate; and 0.2 wt % of colloidal silicon dioxide. In some embodiments, the pharmaceutical composition comprises 127-129 mg of microcrystalline cellulose. In some embodiments, the pharmaceutical composition comprises 127-129 mg of mannitol. In some embodiments, the pharmaceutical composition comprises 16-18 mg of croscarmellose sodium. In some embodiments, the pharmaceutical composition comprises 5-6 mg of magnesium stearate. In some embodiments, the pharmaceutical composition comprises 1.0-1.2 mg of colloidal silicon dioxide. In some embodiments, the pharmaceutical composition comprises about 280 mg of a solid dispersion comprising a polymer and Compound 1. In some embodiments, the pharmaceutical composition comprises about 280 mg of a solid dispersion comprising a polymer and Compound 1; 127-129 mg of microcrystalline cellulose; 127-129 mg of mannitol; 16-18 mg of croscarmellose sodium; 5-6 mg of magnesium stearate; and 1.0-1.2 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; 127-129 mg of microcrystalline cellulose; 127-129 mg of mannitol; 16-18 mg of croscarmellose sodium; 5-6 mg of magnesium stearate; and 1.0-1.2 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; about 128.2 mg of microcrystalline cellulose; about 128.2 mg of mannitol; about 16.8 mg of croscarmellose sodium; about 5.6 mg of magnesium stearate; and about 1.1 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; about 128.24 mg of microcrystalline cellulose; about 128.24 mg of mannitol; about 16.8 mg of croscarmellose sodium; about 5.6 mg of magnesium stearate; and about 1.12 mg of colloidal silicon dioxide. In some embodiments, the pharmaceutical composition comprises 280 mg of a solid dispersion comprising a polymer (e.g., HPMCAS) and Compound 1, wherein the solid dispersion comprises 210 mg of the polymer (e.g., HPMCAS) and 70 mg of Compound 1; 128.2 mg of microcrystalline cellulose; 128.2 mg of mannitol; 16.8 mg of croscarmellose sodium; 5.6 mg of magnesium stearate; and 1.1 mg of colloidal silicon dioxide. In some embodiments, the pharmaceutical composition comprises 280 mg of a solid dispersion comprising a polymer (e.g., HPMCAS) and Compound 1, wherein the solid dispersion comprises 210 mg of the polymer (e.g., HPMCAS) and 70 mg of Compound 1; 128.24 mg of microcrystalline cellulose; 128.24 mg of mannitol; 16.8 mg of croscarmellose sodium; 5.6 mg of magnesium stearate; and 1.12 mg of colloidal silicon dioxide. In some embodiments, the pharmaceutical composition comprises 280 mg of a solid dispersion comprising a polymer and Compound 1; 128.2 mg of microcrystalline cellulose; 128.2 mg of mannitol; 16.8 mg of croscarmellose sodium; 5.6 mg of magnesium stearate; and 1.1 mg of colloidal silicon dioxide. In some embodiments, the pharmaceutical composition comprises 280 mg of a solid dispersion comprising a polymer and Compound 1; 128.24 mg of microcrystalline cellulose; 128.24 mg of mannitol; 16.8 mg of croscarmellose sodium; 5.6 mg of magnesium stearate; and 1.12 mg of colloidal silicon dioxide. In some embodiments the pharmaceutical composition is suitable for tableting by direct compression. In some embodiments, Compound 1, or a pharmaceutically acceptable salt thereof, is administered in an amount of 20-75 mg per day or 60-70 mg per day. In some embodiments, Compound 1, or a pharmaceutically acceptable salt thereof, is administered in an amount of about 70 mg per day. In some embodiments, Compound 1, or a pharmaceutically acceptable salt thereof, is administered in an amount of 70 mg per day.

### Compound, Pharmaceutically Acceptable Salts, and Compositions for Use

In another aspect, the disclosure relates to Compound 1, or a pharmaceutically acceptable salt or pharmaceutical composition thereof, for use in a method of treating pain in a subject in accordance with the method described herein (including any embodiment thereof).

In another aspect, the disclosure relates to Compound 1, or a pharmaceutically acceptable salt or pharmaceutical composition thereof, for use in a method of treating chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia in a subject.

In another aspect, the disclosure relates to Compound 1, or a pharmaceutically acceptable salt or pharmaceutical composition thereof, for use in a method of treating chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia in a subject.

In another aspect, the disclosure relates to Compound 1, or a pharmaceutically acceptable salt or pharmaceutical composition thereof, for use in a method of treating gut pain in a subject, wherein gut pain comprises inflammatory bowel disease pain, Crohn's disease pain, irritable bowel syndrome, endometriosis, polycystic ovarian disease, salpingitis, cervicitis or interstitial cystitis pain.

In another aspect, the disclosure relates to Compound 1, or a pharmaceutically acceptable salt or pharmaceutical composition thereof, for use in a method of treating neuropathic pain in a subject. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the disclosure relates to Compound 1, or a pharmaceutically acceptable salt or pharmaceutical composition thereof, for use in a method of treating neuropathic pain in a subject, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the disclosure relates to Compound 1, or a pharmaceutically acceptable salt or pharmaceutical composition thereof, for use in a method of treating musculoskeletal pain in a subject. In some aspects, the musculoskeletal pain comprises osteoarthritis pain.

In another aspect, the disclosure relates to Compound 1, or a pharmaceutically acceptable salt or pharmaceutical composition thereof, for use in a method of treating musculoskeletal pain in a subject, wherein musculoskeletal pain comprises osteoarthritis pain, back pain, cold pain, burn pain or dental pain.

In another aspect, the disclosure relates to Compound 1, or a pharmaceutically acceptable salt or pharmaceutical composition thereof, for use in a method of treating inflammatory pain in a subject, wherein inflammatory pain comprises rheumatoid arthritis pain, ankylosing spondylitis or vulvodynia.

In another aspect, the disclosure relates to Compound 1, or a pharmaceutically acceptable salt or pharmaceutical composition thereof, for use in a method of treating inflammatory pain in a subject, wherein inflammatory pain comprises rheumatoid arthritis pain.

In another aspect, the disclosure relates to Compound 1, or a pharmaceutically acceptable salt or pharmaceutical composition thereof, for use in a method of treating idiopathic pain in a subject, wherein idiopathic pain comprises fibromyalgia pain.

In another aspect, the disclosure relates to Compound 1, or a pharmaceutically acceptable salt or pharmaceutical composition thereof, for use in a method of treating idiopathic pain in a subject, wherein idiopathic pain comprises reflex sympathetic dystrophy pain.

In another aspect, the disclosure relates to Compound 1, or a pharmaceutically acceptable salt or pharmaceutical composition thereof, for use in a method of treating pathological cough in a subject.

In another aspect, the disclosure relates to Compound 1, or a pharmaceutically acceptable salt or pharmaceutical composition thereof, for use in a method of treating acute pain in a subject. In some aspects, the acute pain comprises acute post-operative pain.

In another aspect, the disclosure relates to Compound 1, or a pharmaceutically acceptable salt or pharmaceutical composition thereof, for use in a method of treating postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) in a subject.

In another aspect, the disclosure relates to Compound 1, or a pharmaceutically acceptable salt or pharmaceutical composition thereof, for use in a method of treating bunionectomy pain in a subject.

In another aspect, the disclosure relates to Compound 1, or a pharmaceutically acceptable salt or pharmaceutical composition thereof, for use in a method of treating shoulder arthroplasty pain or shoulder arthroscopy pain in a subject.

In another aspect, the disclosure relates to Compound 1, or a pharmaceutically acceptable salt or pharmaceutical composition thereof, for use in a method of treating herniorrhaphy pain in a subject.

In another aspect, the disclosure relates to Compound 1, or a pharmaceutically acceptable salt or pharmaceutical composition thereof, for use in a method of treating abdominoplasty pain in a subject.

In another aspect, the disclosure relates to Compound 1, or a pharmaceutically acceptable salt or pharmaceutical composition thereof, for use in a method of treating visceral pain in a subject. In some aspects, the visceral pain comprises visceral pain from abdominoplasty.

In another aspect, the disclosure relates to Compound 1, or a pharmaceutically acceptable salt or pharmaceutical composition thereof, for use in a method of treating a neurodegenerative disease in a subject. In some aspects, the neurodegenerative disease comprises multiple sclerosis. In some aspects, the neurodegenerative disease comprises Pitt Hopkins Syndrome (PTHS).

In another aspect, the disclosure relates to Compound 1, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the disclosure relates to Compound 1, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Naᵥ1.8.

In another aspect, the disclosure relates to Compound 1, or a pharmaceutically acceptable salt or pharmaceutical composition thereof, for use in a method of treating acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility in a subject.

In another aspect, the disclosure relates to Compound 1, or a pharmaceutically acceptable salt or pharmaceutical composition thereof, for use in a method of treating femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain in a subject.

In another aspect, the disclosure relates to Compound 1, or a pharmaceutically acceptable salt or pharmaceutical composition thereof, for use in a method of treating trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain in a subject.

Manufacture ofMedicaments

In another aspect, the disclosure relates to the use of Compound 1, or a pharmaceutically acceptable salt or pharmaceutical composition thereof, for the manufacture of a medicament for treating pain in a subject in accordance with the method described herein (including any embodiment thereof).

In yet another aspect, the disclosure relates to the use of Compound 1, a pharmaceutically acceptable salt thereof, or a pharmaceutical composition thereof for the manufacture of a medicament for use in treating chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia in a subject.

In yet another aspect, the disclosure relates to the use of Compound 1, a pharmaceutically acceptable salt thereof, or a pharmaceutical composition thereof for the manufacture of a medicament for use in treating chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia in a subject.

In yet another aspect, the disclosure relates to the use of Compound 1, pharmaceutically acceptable salt, or pharmaceutical composition described herein for the manufacture of a medicament for use in treating gut pain in a subject, wherein gut pain comprises inflammatory bowel disease pain, Crohn's disease pain, irritable bowel syndrome, endometriosis, polycystic ovarian disease, salpingitis, cervicitis or interstitial cystitis pain.

In yet another aspect, the disclosure relates to the use of Compound 1, a pharmaceutically acceptable salt thereof, or a pharmaceutical composition thereof for the manufacture of a medicament for use in treating neuropathic pain in a subject. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the disclosure relates to the use of Compound 1, a pharmaceutically acceptable salt thereof, or a pharmaceutical composition thereof for the manufacture of a medicament for use in a treating neuropathic pain in a subject, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the disclosure relates to the use of Compound 1, a pharmaceutically acceptable salt thereof, or a pharmaceutical composition thereof for the manufacture of a medicament for use in treating musculoskeletal pain in a subject. In some aspects, the musculoskeletal pain comprises osteoarthritis pain.

In yet another aspect, the disclosure relates to the use of Compound 1, a pharmaceutically acceptable salt thereof, or a pharmaceutical composition thereof for the manufacture of a medicament for use in treating musculoskeletal pain in a subject, wherein musculoskeletal pain comprises osteoarthritis pain, back pain, cold pain, burn pain or dental pain.

In yet another aspect, the disclosure relates to the use of Compound 1, a pharmaceutically acceptable salt thereof, or a pharmaceutical composition thereof for the manufacture of a medicament for use in treating inflammatory pain in a subject, wherein inflammatory pain comprises rheumatoid arthritis pain, ankylosing spondylitis or vulvodynia.

In yet another aspect, the disclosure relates to the use of Compound 1, a pharmaceutically acceptable salt thereof, or a pharmaceutical composition thereof for the manufacture of a medicament for use in treating inflammatory pain in a subject, wherein inflammatory pain comprises rheumatoid arthritis pain.

In yet another aspect, the disclosure relates to the use of Compound 1, a pharmaceutically acceptable salt thereof, or a pharmaceutical composition thereof for the manufacture of a medicament for use in treating idiopathic pain in a subject, wherein idiopathic pain comprises fibromyalgia pain.

In yet another aspect, the disclosure relates to the use of Compound 1, a pharmaceutically acceptable salt thereof, or a pharmaceutical composition thereof for the manufacture of a medicament for use in treating idiopathic pain in a subject, wherein idiopathic pain comprises reflex sympathetic dystrophy pain.

In yet another aspect, the disclosure relates to the use of Compound 1, a pharmaceutically acceptable salt thereof, or a pharmaceutical composition thereof for the manufacture of a medicament for use in treating pathological cough in a subject.

In yet another aspect, the disclosure relates to the use of Compound 1, a pharmaceutically acceptable salt thereof, or a pharmaceutical composition thereof for the manufacture of a medicament for use in treating acute pain in a subject. In some aspects, the acute pain comprises acute post-operative pain.

In yet another aspect, the disclosure relates to the use of Compound 1, a pharmaceutically acceptable salt thereof, or a pharmaceutical composition thereof for the manufacture of a medicament for use in treating postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) in a subject.

In yet another aspect, the disclosure relates to the use of Compound 1, a pharmaceutically acceptable salt thereof, or a pharmaceutical composition thereof for the manufacture of a medicament for use in treating herniorrhaphy pain in a subject.

In yet another aspect, the disclosure relates to the use of Compound 1, a pharmaceutically acceptable salt thereof, or a pharmaceutical composition thereof for the manufacture of a medicament for use in treating bunionectomy pain in a subject.

In yet another aspect, the disclosure relates to the use of Compound 1, a pharmaceutically acceptable salt thereof, or a pharmaceutical composition thereof for the manufacture of a medicament for use in treating shoulder arthroplasty pain or shoulder arthroscopy pain in a subject.

In yet another aspect, the disclosure relates to the use of Compound 1, a pharmaceutically acceptable salt thereof, or a pharmaceutical composition thereof for the manufacture of a medicament for use in treating abdominoplasty pain in a subject.

In yet another aspect, the disclosure relates to the use of Compound 1, a pharmaceutically acceptable salt thereof, or a pharmaceutical composition thereof for the manufacture of a medicament for use in treating visceral pain in a subject. In some aspects, the visceral pain comprises visceral pain from abdominoplasty.

In another aspect, the disclosure relates to the use of Compound 1, or a pharmaceutically acceptable salt or pharmaceutical composition thereof, for the manufacture of a medicament for use in treating a neurodegenerative disease in a subject. In some aspects, the neurodegenerative disease comprises multiple sclerosis. In some aspects, the neurodegenerative disease comprises Pitt Hopkins Syndrome (PTHS).

In yet another aspect, the disclosure relates to the use of Compound 1, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the disclosure relates to the use of Compound 1, a pharmaceutically acceptable salt thereof, or a pharmaceutical composition thereof for the manufacture of a medicament for use in treating acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility in a subject.

In another aspect, the disclosure relates to the use of Compound 1, a pharmaceutically acceptable salt thereof, or a pharmaceutical composition thereof for the manufacture of a medicament for use in treating femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain in a subject.

In another aspect, the disclosure relates to the use of Compound 1, a pharmaceutically acceptable salt thereof, or a pharmaceutical composition thereof for the manufacture of a medicament for use in treating trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain in a subject.

### Pharmaceutically Acceptable Salts, Pharmaceutical Compositions, Dosage Forms and Routes of Administration

### Pharmaceutically Acceptable Salts

The methods described and claimed herein comprise administering to a subject Compound 1, or a pharmaceutically acceptable salt thereof. As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” of Compound 1 includes any non-toxic salt that, upon administration to a recipient, is capable of providing, either directly or indirectly, Compound 1 or an inhibitorily active metabolite or residue thereof (e.g., the parent compound of a prodrug). The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of Compound 1 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, and ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Compositions

In the methods described and claimed herein, Compound 1, or a pharmaceutically acceptable salt thereof, may be administered in the form of a pharmaceutical composition comprising Compound 1, or a pharmaceutically acceptable salt thereof, and a pharmaceutically acceptable carrier, adjuvant or vehicle.

The term “pharmaceutically acceptable carrier, adjuvant, or vehicle” includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with Compound 1, or a pharmaceutically acceptable salt thereof, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such a propylene glycol or polyethylene glycol, esters such as ethyl oleate and ethyl laurate, agar, buffering agents such as magnesium hydroxide and aluminum hydroxide, alginic acid, pyrogen-free water, isotonic saline, Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Dosage Forms and Routes of Administration

The methods described and claimed herein may involve the administration of Compound 1, or a pharmaceutically acceptable salt thereof, by any route of administration effective for treating one or more of the pain diseases recited herein. Compound 1, or a pharmaceutically acceptable salt thereof, may be formulated in dosage unit form for ease of administration and uniformity of dosage. The term “dosage unit form,” as used herein, refers to a physically discrete unit of agent appropriate for the subject to be treated.

Compound 1, or pharmaceutically acceptable salt thereof, can be administered to humans and other animals orally, rectally, parenterally, intracisternally, intravaginally, intraperitoneally, topically (as by powders, ointments, or drops), buccally, as an oral or nasal spray, or the like, depending on the condition being treated.

Liquid dosage forms for oral administration include, but are not limited to, pharmaceutically acceptable emulsions, microemulsions, solutions, suspensions, syrups and elixirs. In addition to Compound 1, or a pharmaceutically acceptable salt thereof, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the therapeutic effect of Compound 1, it may be desirable to slow the absorption of the compound, or a pharmaceutically acceptable salt thereof, from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing Compound 1, or a pharmaceutically acceptable salt thereof, with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, Compound 1, or a pharmaceutically acceptable salt thereof, is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or, in a certain part of the intestinal tract, optionally, in a delayed manner. Examples of embedding compositions that can be used include polymeric substances and waxes.

The active compound or salt can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of Compound 1, or a pharmaceutically acceptable salt thereof, include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In another aspect, the disclosure relates to a pharmaceutical composition comprising: 45-55 wt % of a solid dispersion comprising a polymer and Compound 1; 42-50 wt % of one or more fillers; 2-4 wt % of a disintegrant; and 0.5-1.5 wt % of a lubricant. In some embodiments, the one or more fillers comprise microcrystalline cellulose. In some embodiments, the disintegrant is croscarmellose sodium. In some embodiments, the lubricant is magnesium stearate. In some embodiments, the pharmaceutical composition comprises: 45-55 wt % of a solid dispersion comprising a polymer and Compound 1; 42-50 wt % of microcrystalline cellulose; 2-4 wt % of croscarmellose sodium; and 0.5-1.5 wt % of magnesium stearate. In some embodiments, the pharmaceutical composition is a tablet core composition. In some embodiments, the tablet core composition is coated with a tablet coating (e.g., Opadry Blue). In some embodiments, the pharmaceutical composition comprises about 50 mg of Compound 1. In some embodiments, the polymer is HPMCAS. In some embodiments, the solid dispersion comprises 70-80 wt % of the polymer and 20-30 wt % of Compound 1 (e.g., about 75 wt % of the polymer and about 25 wt % of Compound 1). In some embodiments, the pharmaceutical composition comprises about 50 wt % of the solid dispersion; about 46 wt % of microcrystalline cellulose; about 3 wt % of croscarmellose sodium; and about 1.0 wt % of magnesium stearate. In some embodiments, the pharmaceutical composition comprises about 50 wt % of the solid dispersion, wherein the solid dispersion comprises about 75 wt % of the polymer (e.g., HPMCAS) and about 25 wt % of Compound 1; about 46 wt % of microcrystalline cellulose; about 3 wt % of croscarmellose sodium; and about 1.0 wt % of magnesium stearate. In some embodiments, the pharmaceutical composition comprises 50 wt % of the solid dispersion, wherein the solid dispersion comprises about 75 wt % of the polymer (e.g., HPMCAS) and about 25 wt % of Compound 1; 46 wt % of microcrystalline cellulose; 3 wt % of croscarmellose sodium; and 1.0 wt % of magnesium stearate. In some embodiments, the pharmaceutical composition comprises 50 wt % of the solid dispersion, wherein the solid dispersion comprises 75 wt % of the polymer (e.g., HPMCAS) and 25 wt % of Compound 1; 46 wt % of microcrystalline cellulose; 3 wt % of croscarmellose sodium; and 1.0 wt % of magnesium stearate. In some embodiments, the pharmaceutical composition comprises about 184 mg of microcrystalline cellulose. In some embodiments, the pharmaceutical composition comprises about 12 mg of croscarmellose sodium. In some embodiments, the pharmaceutical composition comprises about 4 mg of magnesium stearate. In some embodiments, the pharmaceutical composition comprises about 200 mg of a solid dispersion comprising a polymer and Compound 1. In some embodiments, the pharmaceutical composition comprises about 200 mg of a solid dispersion comprising a polymer and Compound 1; 184 mg of microcrystalline cellulose; 12 mg of croscarmellose sodium; and 4 mg of magnesium stearate. In some embodiments, the pharmaceutical composition comprises about 200 mg of a solid dispersion comprising a polymer (e.g., HPMCAS) and Compound 1, wherein the solid dispersion comprises about 150 mg of the polymer (e.g., HPMCAS) and about 50 mg of Compound 1; about 184 mg of microcrystalline cellulose; about 12 mg of croscarmellose sodium; and about 4 mg of magnesium stearate. In some embodiments, the pharmaceutical composition comprises 200 mg of a solid dispersion comprising a polymer (e.g., HPMCAS) and Compound 1, wherein the solid dispersion comprises 150 mg of the polymer (e.g., HPMCAS) and 50 mg of Compound 1; 184 mg of microcrystalline cellulose; 12 mg of croscarmellose sodium; and 4 mg of magnesium stearate.

In some embodiments, the pharmaceutical composition comprises an intragranular blend and extragranular blend. In some embodiments, the intragranular blend comprises 45-55 wt % of a solid dispersion comprising a polymer and Compound 1; 20-25 wt % of a filler; 1-2 wt % of a disintegrant; and 0-0.2 wt % of a lubricant; and the extragranular blend comprises 20-25 wt % of a filler; 1-2 wt % of a disintegrant; and 0.8-1.0 wt % of a lubricant, wherein such weight percentages are based on the combined weights of the intragranular and extragranular blends. In some embodiments, the intragranular blend comprises 45-55 wt % of a solid dispersion comprising a polymer and Compound 1; 20-25 wt % of microcrystalline cellulose; 1-2 wt % of croscarmellose sodium; and 0-0.2 wt % of magnesium stearate; and the extragranular blend comprises 20-25 wt % of microcrystalline cellulose; 1-2 wt % of croscarmellose sodium; and 0.8-1.0 wt % of magnesium stearate, wherein such weight percentages are based on the combined weights of the intragranular and extragranular blends. In some embodiments, the intragranular blend comprises about 50 wt % of a solid dispersion comprising a polymer and Compound 1, wherein the solid dispersion comprises about 75 wt % of the polymer (e.g., HPMCAS) and about 25 wt % of Compound 1; about 23.4 wt % of microcrystalline cellulose; about 1.5 wt % of croscarmellose sodium; and about 0.1 wt % of magnesium stearate; and the extragranular blend comprises about 22.6 wt % of microcrystalline cellulose; about 1.5 wt % of croscarmellose sodium; and about 0.9 wt % of magnesium stearate, wherein such weight percentages are based on the combined weights of the intragranular and extragranular blends. In some embodiments, the intragranular blend comprises 50 wt % of a solid dispersion comprising a polymer and Compound 1, wherein the solid dispersion comprises 75 wt % of the polymer (e.g., HPMCAS) and 25 wt % of Compound 1; 23.4 wt % of microcrystalline cellulose; 1.5 wt % of croscarmellose sodium; and 0.1 wt % of magnesium stearate; and the extragranular blend comprises 22.6 wt % of microcrystalline cellulose; 1.5 wt % of croscarmellose sodium; and 0.9 wt % of magnesium stearate, wherein such weight percentages are based on the combined weights of the intragranular and extragranular blends. In some embodiments, the intragranular blend comprises about 200 mg of the solid dispersion, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate. In some embodiments, the intragranular blend comprises about 200 mg of the solid dispersion, wherein the solid dispersion comprises about 150 mg of the polymer (e.g., HPMCAS) and about 50 mg of Compound 1, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate. In some embodiments, the intragranular blend comprises 200 mg of the solid dispersion, wherein the solid dispersion comprises 150 mg of the polymer (e.g., HPMCAS) and 50 mg of Compound 1, 93.6 mg of microcrystalline cellulose, 6.0 mg of croscarmellose sodium, and 0.4 mg of magnesium stearate; and the extragranular blend comprises 90.4 mg of microcrystalline cellulose, 6.0 mg of croscarmellose sodium, and 3.6 mg of magnesium stearate. In some embodiments, the intragranular blend comprises about 0.4 mg of magnesium stearate, and the extragranular blend comprises about 3.6 mg of magnesium stearate. In some embodiments, the intragranular blend comprises 0.4 mg of magnesium stearate, and the extragranular blend comprises 3.6 mg of magnesium stearate.

In another aspect, the disclosure relates to a pharmaceutical composition comprising: 45-55 wt % of a solid dispersion comprising a polymer and Compound 1; 40-50 wt % of one or more fillers; 2-4 wt % of a disintegrant; 0.5-1.5 wt % of a lubricant; and 0.05-0.8 wt % (e.g., 0.1-0.3 wt %) of a glidant. In some embodiments, the pharmaceutical composition comprises: 45-55 wt % of a solid dispersion comprising a polymer and Compound 1; 40-50 wt % of one or more fillers; 2-4 wt % of a disintegrant; and 0.5-1.5 wt % of a lubricant. In some embodiments, the one or more fillers comprise microcrystalline cellulose and mannitol. In some embodiments, the disintegrant is croscarmellose sodium. In some embodiments, the lubricant is magnesium stearate. In some embodiments, the glidant is colloidal silicon dioxide. In some embodiments, the pharmaceutical composition comprises: 45-55 wt % of a solid dispersion comprising a polymer and Compound 1; 20-25 wt % of microcrystalline cellulose; 20-25 wt % of mannitol; 2-4 wt % of croscarmellose sodium; 0.5-1.5 wt % of magnesium stearate; and 0.1-0.3 wt % of colloidal silicon dioxide. In some embodiments, the pharmaceutical composition comprises: 45-55 wt % of a solid dispersion comprising a polymer and Compound 1; 20-25 wt % of microcrystalline cellulose; 20-25 wt % of mannitol; 2-4 wt % of croscarmellose sodium; and 0.5-1.5 wt % of magnesium stearate. In some embodiments, the pharmaceutical composition is a tablet core composition. In some embodiments, the tablet core composition is coated with a tablet coating (e.g., Opadry Blue). In some embodiments, the pharmaceutical composition comprises about 70 mg of Compound 1. In some embodiments, the polymer is HPMCAS. In some embodiments, the solid dispersion comprises 70-80 wt % of the polymer and 20-30 wt % of Compound 1 (e.g., about 75 wt % of the polymer and about 25 wt % of Compound 1). In some embodiments, the pharmaceutical composition comprises about 50 wt % of the solid dispersion; about 22.9 wt % of microcrystalline cellulose; about 22.9 wt % of mannitol; about 3 wt % of croscarmellose sodium; about 1.0 wt % of magnesium stearate; and about 0.2 wt % of colloidal silicon dioxide. In some embodiments, the pharmaceutical composition comprises about 50 wt % of the solid dispersion; about 22.9 wt % of microcrystalline cellulose; about 22.9 wt % of mannitol; about 3 wt % of croscarmellose sodium; and about 1.0 wt % of magnesium stearate. In some embodiments, the pharmaceutical composition comprises about 50 wt % of a solid dispersion comprising a polymer (e.g., HPMCAS) and Compound 1, wherein the solid dispersion comprises about 75 wt % of the polymer (e.g., HPMCAS) and about 25 wt % of Compound 1; about 22.9 wt % of microcrystalline cellulose; about 22.9 wt % of mannitol; about 3 wt % of croscarmellose sodium; about 1.0 wt % of magnesium stearate; and about 0.2 wt % of colloidal silicon dioxide. In some embodiments, the pharmaceutical composition comprises 50 wt % of of a solid dispersion comprising a polymer (e.g., HPMCAS) and Compound 1, wherein the solid dispersion comprises 75 wt % of the polymer (e.g., HPMCAS) and 25 wt % of Compound 1; 22.9 wt % of microcrystalline cellulose; 22.9 wt % of mannitol; 3 wt % of croscarmellose sodium; 1.0 wt % of magnesium stearate; and 0.2 wt % of colloidal silicon dioxide. In some embodiments, the pharmaceutical composition comprises 127-129 mg of microcrystalline cellulose. In some embodiments, the pharmaceutical composition comprises 127-129 mg of mannitol. In some embodiments, the pharmaceutical composition comprises 16-18 mg of croscarmellose sodium. In some embodiments, the pharmaceutical composition comprises 5-6 mg of magnesium stearate. In some embodiments, the pharmaceutical composition comprises 1.0-1.2 mg of colloidal silicon dioxide. In some embodiments, the pharmaceutical composition comprises about 280 mg of a solid dispersion comprising a polymer and Compound 1. In some embodiments, the pharmaceutical composition comprises about 280 mg of a solid dispersion comprising a polymer and Compound 1; 127-129 mg of microcrystalline cellulose; 127-129 mg of mannitol; 16-18 mg of croscarmellose sodium; 5-6 mg of magnesium stearate; and 1.0-1.2 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; 127-129 mg of microcrystalline cellulose; 127-129 mg of mannitol; 16-18 mg of croscarmellose sodium; 5-6 mg of magnesium stearate; and 1.0-1.2 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; about 128.2 mg of microcrystalline cellulose; about 128.2 mg of mannitol; about 16.8 mg of croscarmellose sodium; about 5.6 mg of magnesium stearate; and about 1.1 mg of colloidal silicon dioxide. In some embodiments, the pharmaceutical composition comprises about 280 mg of a solid dispersion comprising a polymer (e.g., HPMCAS) and Compound 1, wherein the solid dispersion comprises about 210 mg of the polymer (e.g., HPMCAS) and about 70 mg of Compound 1; about 128.24 mg of microcrystalline cellulose; about 128.24 mg of mannitol; about 16.8 mg of croscarmellose sodium; about 5.6 mg of magnesium stearate; and about 1.12 mg of colloidal silicon dioxide. In some embodiments, the pharmaceutical composition comprises 280 mg of a solid dispersion comprising a polymer (e.g., HPMCAS) and Compound 1, wherein the solid dispersion comprises 210 mg of the polymer (e.g., HPMCAS) and 70 mg of Compound 1; 128.2 mg of microcrystalline cellulose; 128.2 mg of mannitol; 16.8 mg of croscarmellose sodium; 5.6 mg of magnesium stearate; and 1.1 mg of colloidal silicon dioxide. In some embodiments, the pharmaceutical composition comprises 280 mg of a solid dispersion comprising a polymer (e.g., HPMCAS) and Compound 1, wherein the solid dispersion comprises 210 mg of the polymer (e.g., HPMCAS) and 70 mg of Compound 1; 128.24 mg of microcrystalline cellulose; 128.24 mg of mannitol; 16.8 mg of croscarmellose sodium; 5.6 mg of magnesium stearate; and 1.12 mg of colloidal silicon dioxide. In some embodiments, the pharmaceutical composition comprises 280 mg of a solid dispersion comprising a polymer and Compound 1; 128.2 mg of microcrystalline cellulose; 128.2 mg of mannitol; 16.8 mg of croscarmellose sodium; 5.6 mg of magnesium stearate; and 1.1 mg of colloidal silicon dioxide. In some embodiments, the pharmaceutical composition comprises 280 mg of a solid dispersion comprising a polymer and Compound 1; 128.24 mg of microcrystalline cellulose; 128.24 mg of mannitol; 16.8 mg of croscarmellose sodium; 5.6 mg of magnesium stearate; and 1.12 mg of colloidal silicon dioxide. In some embodiments the pharmaceutical composition is suitable for tableting by direct compression.

### Additional Therapeutic Agents

It will also be appreciated that Compound 1, pharmaceutically acceptable salts of Compound 1, and pharmaceutically acceptable compositions of Compound 1 can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin, naphthylalkanones such as Nabumetone, oxicams such as Piroxicam, para-aminophenol derivatives, such as Acetaminophen, propionic acids such as Fenoprofen, Flurbiprofen, Ibuprofen, Ketoprofen, Naproxen, Naproxen sodium, Oxaprozin, salicylates such as Aspirin, Choline magnesium trisalicylate, Diflunisal, fenamates such as meclofenamic acid, Mefenamic acid, and pyrazoles such as Phenylbutazone), or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (-)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER@), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (l[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite 0-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-4-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Naᵥ1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Naᵥ1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone. [00251](39) a Navl.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO2021/257420, WO2021/257418, WO2022/263498, WO2022/235558, WO2022/235859, WO2023/138599, WO2023/211990, WO2023/211990, WO2023/238065, CN112390745, CN111808019, CN112225695, CN112457294, CN112300051, CN112300069, CN112441969, and CN114591293, the entire contents of each application hereby incorporated by reference;
(39a) a Navl.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Naᵥ1.7 and Naᵥ1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof;
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011/Zynrelefr™).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGR1 inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NE01940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Naᵥ1.7 blocker such as ST-2427, ST-2578 and those disclosed in WO2010/129864, WO2015/157559, WO2017/059385, WO2018/183781, WO2018/183782, WO2020/072835, and/or WO2022/036297 the entire contents of each application hereby incorporated by reference.

In another embodiment, the additional therapeutic agent is selected from ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, 5-600918, S-637880, RQ-00434739, KLS-2031, MEDI 7352, and XT-150.

In another embodiment, the additional therapeutic agent is selected from Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 and ACD440.

In another embodiment, the additional therapeutic agent is selected from HRS4800, ODM-111/JMKX000623, LX9211, LY3556050, LY3857210, CFTX01554/CFTX-1554, MEDI7352, MEDI0618, BAY3178275, BAY2395840, GSK3858279, STC-004, HALNEURON, OLP-1002, ATXO1, ANP230, CC-8464, iN1011-N17, ST-2427, MSD199, FZ008, VYNAV-01, BL-017881, Profervia (Cilnidipine), LS-04, vixotrigine, FX301/PCRX-301, PF-04531083, PF-01247324, and DSP-3905.

In another embodiment, the additional therapeutic agent is a sodium channel inhibitor (also known as a sodium channel blocker), such as the Naᵥ1.7 and Naᵥ1.8 blockers identified above. The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

### Synthesis of Compound 1

In another aspect, the disclosure relates to a process for preparing Compound 1, comprising transforming a compound of formula 5:

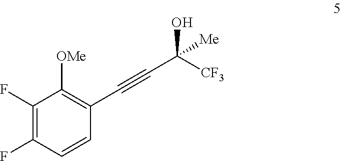 [SMILES: COc1c(C#C[C@@](C)(O)C(F)(F)F)ccc(F)c1F]

### to Compound 1.

In some embodiments, transforming a compound of formula 5 to Compound 1 comprises transforming the compound of formula 5 to a compound of formula 11:

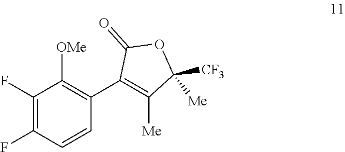 [SMILES: COc1c(C2=C(C)[C@](C)(C(F)(F)F)OC2=O)ccc(F)c1F]

and transforming the compound of formula 11 to Compound 1. In some embodiments, transforming the compound of formula 5 to the compound of formula 11 comprises treating the compound of formula 5 with methyllithium and carbon dioxide in the presence of cerium (III) chloride and lithium chloride. In other embodiments, transforming the compound of formula 5 to the compound of formula 11 comprises treating the compound of formula 5 with methylmagnesium chloride and carbon dioxide in the presence of copper (I) chloride and lithium chloride.

In some embodiments, the compound of formula 5 is obtained by transforming a compound of formula 4:

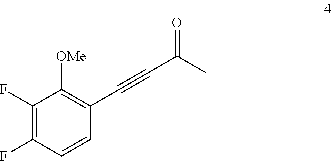

to the compound of formula 5. In some embodiments, transforming the compound of formula 4 to the compound of formula 5 comprises treating the compound of formula 4 with trimethyl(trifluoromethyl)silane in the presence of catalytic CAF-1:

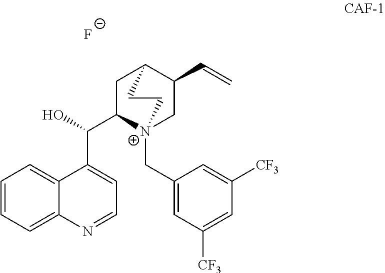

followed by treatment with TBAF.

In some embodiments, the compound of formula 4 is obtained by transforming a compound of formula 3:

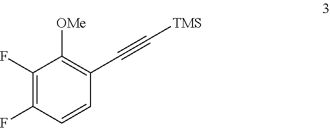 [SMILES: COc1c(C#C[Si](C)(C)C)ccc(F)c1F]

to the compound of formula 4. In some embodiments, transforming the compound of formula 3 to the compound of formula 4 comprises treating the compound of formula 3 with aluminum (III) chloride and acetyl chloride.

In some embodiments, the compound of formula 5 is obtained by transforming a compound of formula 4′:

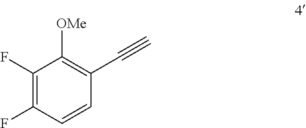

to the compound of formula 5. In some embodiments, transforming the compound of formula 4′ to the compound of formula 5 comprises treating the compound of formula 4′ with dimethylzinc, titanium (IV) isopropoxide, and 1,1,1-trifluoroacetone in the presence of catalytic CL1:

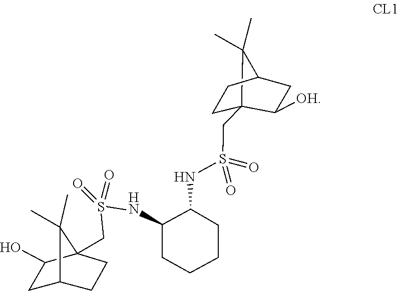 [SMILES: CC1(C)C2CCC1(CS(=O)(=O)N[C@@H]1CCCC[C@H]1NS(=O)(=O)CC13CCC(CC1O)C3(C)C)C(O)C2]

In some embodiments, the compound of formula 4′ is obtained by transforming a compound of formula 3:

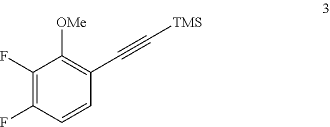 [SMILES: COc1c(C#C[Si](C)(C)C)ccc(F)c1F]

to the compound of formula 4′. In some embodiments, transforming the compound of formula 3 to the compound of formula 4′ comprises treating the compound of formula 3 with potassium carbonate.

In some embodiments, the compound of formula 3 is obtained by transforming a compound of formula:

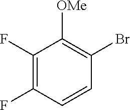 [SMILES: COc1c(Br)ccc(F)c1F]

to the compound of formula 3. In some embodiment, transforming said compound to the compound of formula 3 comprises treating said compound with trimethylsilylacetylene and triethylamine in the presence of catalytic bis(triphenylphosphine)palladium(II) dichloride and copper(I) iodide.

In some embodiments, the compound of formula 11 is transformed to Compound 1 by the methods described in Scheme B of International Publication No. WO 2024/123815, which is incorporated by reference. For completeness, the relevant portion of that Scheme is reproduced here:

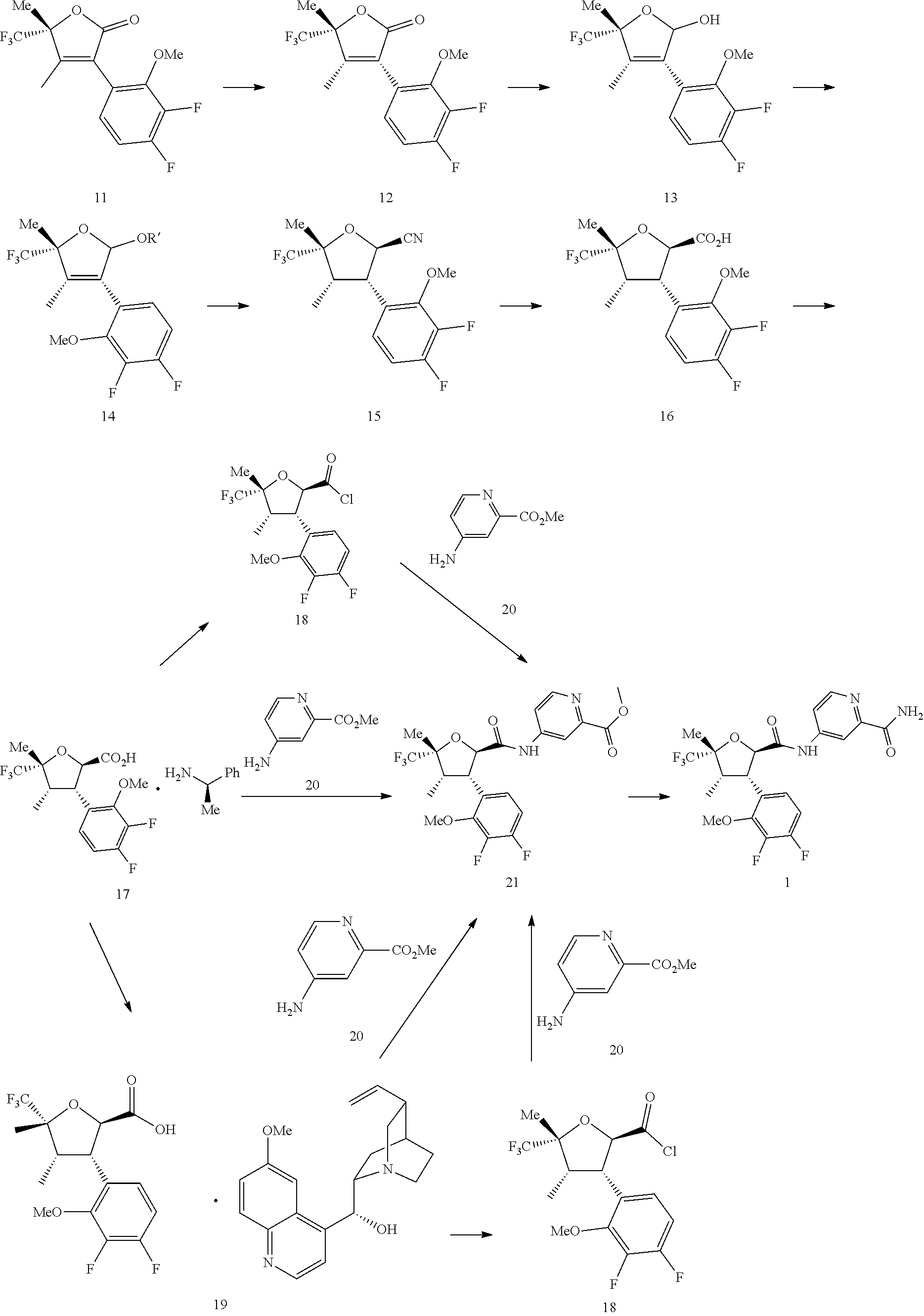

In some embodiments, Compound 1 is prepared by transforming a compound of formula 17:

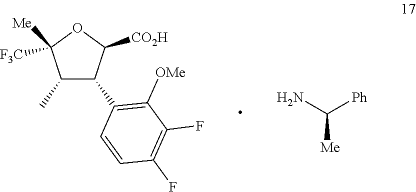 [SMILES: COc1c([C@H]2[C@H](C(=O)O)O[C@@](C)(C(F)(F)F)[C@H]2C)ccc(F)c1F ; C[C@@H](N)c1ccccc1]

### to Compound 1.

In some embodiments, transforming the compound of formula 17 to Compound 1 includes contacting the compound of formula 17 with a compound of formula 20:

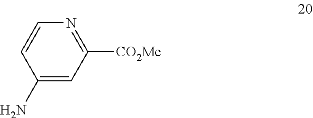 [SMILES: COC(=O)c1cc(N)ccn1]

to afford a compound of formula 21:

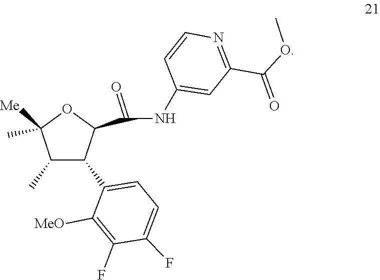

In some embodiments, contacting the compound of formula 17 with a compound of formula 20 is performed in the presence of a coupling reagent and a base. One of ordinary skill in the art will appreciate that many coupling reagents may be compatible with the process of coupling a compound of formula 20 to the compound of formula 16, the compound of formula 19, or the compound of formula 17. For example, in some embodiments, the coupling reagent is 1,1′-carbonyldiimidazole (CDI), 1-ethyl-3-(3-dimethylaminopropyl)carbodiimide (EDCl), diphenylphosphinic chloride (DPPCL), isobutyl chloroformate (IBCF), or propylphosphonic acid anhydride (T3P). In some embodiments, the coupling reagent is T3P. One of ordinary skill in the art will appreciate that many bases may be compatible with the process of coupling a compound of formula 20 to the compound of formula 16, the compound of formula 19, or the compound of formula 17. In some embodiments, the base is a non-nucleophilic base. In some embodiments, the base is selected from trimethylamine, N-methylimidazole, pyridine, 4-methylmorpholine, Hunig's base, DABCO, and NaOH, and the like. In other embodiments, the base may be any C₁-C₄ alkyl tertiary amine, such as triethylamine, ethyldimethyl amine, ethyldipropyl amine and various other alkyl combinations thereof. In some embodiments, the base is triethylamine, 4-methylmorpholine (NMM), or 1-methylimidzole (NMI). In some embodiments, the base is triethylamine.

In some embodiments, the compound of formula 17 is first converted to a free acid, i.e. the compound of formula 16, before being contacted by the compound of formula 20. The compound of formula 17 may be converted to the free acid compound of formula 16 by any method known to one of ordinary skill. In some embodiments, the compound of formula 17 is converted to the free acid compound of formula 16 by treating the compound of formula 17 with an aqueous acid. In some embodiments, the aqueous acid is aqueous hydrochloric acid.

In some embodiments, transforming the compound of formula 17 to Compound 1 includes transforming the compound of formula 17 to a compound of formula 18:

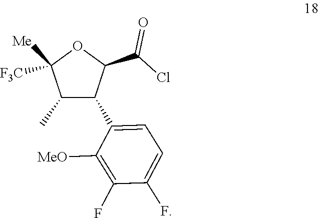 [SMILES: COc1c([C@H]2[C@H](C(=O)Cl)O[C@@](C)(C(F)(F)F)[C@H]2C)ccc(F)c1F]

In some embodiments, the compound of formula 17 is first converted to a free acid, i.e. the compound of formula 16, before being transformed to the compound of formula 18. The compound of formula 17 may be converted to the free acid compound of formula 16 by any method known to one of ordinary skill. In some embodiments, the compound of formula 17 is converted to the free acid compound of formula 16 by treating the compound of formula 17 with an aqueous acid. In some embodiments, the aqueous acid is aqueous hydrochloric acid.

In some embodiments, transforming the compound of formula 17 to the compound of formula 18 includes treating the free acid compound of formula 16 or the compound of formula 17 with a chlorinating reagent. Any chlorinating agent suitable for chlorinating the compound of formula 16, or a salt thereof (i.e. the compound of formula 17), may be used. In some embodiments, the chlorinating agent is thionyl chloride, methanesulfonyl chloride, phosphorus oxychloride, phosphorus pentachloride, phosgene, oxalyl chloride, isobutyl chloroformate (IBCF), pivaloyl chloride (PivCl), or diphenylphosphinic chloride (DPPCl). In some embodiments, the chlorinating agent is oxalyl chloride. In some embodiments, the compound of formula 18 is carried on to the next reaction in the process for preparing Compound 1 without further purification or isolation.

In some embodiments, treating the free acid compound of formula 16 or the compound of formula 17 with a chlorinating reagent is performed in the presence of a non-nucleophilic base. Without being bound to theory, the non-nucleophilic base may be included to scavenge byproduct HCl that is generated during the chlorination reaction. Accordingly, any suitable non-nucleophilic base known by one of ordinary skill in the art may be used. Suitable non-nucleophilic bases are typically tertiary or aromatic amines where the nitrogen of the amine base does not carry an H atom. The non-nucleophilic base may be bulky bases that are non-nucleophilic because of steric hindrance. Examples of suitable bases include Hunig's base, triethylamine, diisopropyl ethylamine, N-methylmorpholine, 1,8-diazabicyclo[5.4.0]undec-7-ene, pyridine, butylamine, or 1,5-diazabicyclo(4.3.0)non-5-ene, or a mixture thereof. In some embodiments, the reaction between the free acid compound of formula 16 or the compound of formula 17 and the chlorinating agent is conducted at a temperature of no more than about 90° C.

In some embodiments, transforming the compound of formula 17 to Compound 1 comprises contacting the compound of formula 18 with the compound of formula 20 to afford the compound of formula 21. In some embodiments, contacting the compound of formula 18 with a compound of formula 20 is performed in the presence of a base. One of ordinary skill in the art will appreciate that many bases would be compatible with the reaction between the compound of formula 18 and the compound of formula 20. In some embodiments, the base is a non-nucleophilic base. In some embodiments, the base is triethylamine. One of ordinary skill in the art will appreciate that many solvents would be compatible with the reaction between the compound of formula 18 and the compound of formula 20. In some embodiments, the solvent is toluene. In some embodiments, the compound of formula 21 is obtained by partitioning the reaction mixture between an organic layer comprising ethyl acetate and toluene and an aqueous layer.

In some embodiments, the compound of formula 21 (prepared either by way of a free acid compound of formula 16, a compound of formula 17, or a compound of formula 18) may be purified, for example, by recrystallizing it from a solvent comprising methanol or water or a mixture thereof. Other suitable combination of two solvents include ethanol/water, toluene/heptane, IPA/water, etc. In any of these combinations, the compound of formula 21 is dissolved in one solvent at boiling or near boiling temperature followed by addition of the second solvent until the solution becomes turbid. The turbid suspension is allowed to cool down to room temperature (or cooled with an ice bath) followed by filtration of the solid.

In some embodiments, transforming a compound of formula 17 to Compound 1 further includes transforming the compound of formula 21 to Compound 1. In some embodiments, transforming the compound of formula 21 to Compound 1 comprises treating the compound of formula 21 with ammonia to afford Compound 1. In some embodiments, the compound of formula 21 may be treated with ammonia in the presence of a suitable solvent. In some embodiments, the suitable solvent is methanol, ethanol, IPA, MeCN, THF, 2-MeTHF, water, or a mixture thereof. In some embodiments, treating the compound of formula 21 with ammonia may be performed in the presence of a weak, non-nucleophilic base. In some embodiments, the base is selected from Mg(OMe)₂, CaCl₂, DIPEA, and K₂CO₃. In some embodiments, the ammonia is in the form of a solution of ammonia in a solvent, ammonia in gas form in which an ammonia gas is bubbled into the reaction mixture, or in the form of ammonium hydroxide or an ammonium salt where ammonia is generated in situ. In some embodiments, the ammonia is in the form of a solution of ammonia in methanol. In some embodiments, the ammonia is in the form of a solution of ammonia in methanol and tetrahydrofuran. In some embodiments, the in situ generation of ammonia includes reacting ammonium hydroxide or the ammonium salt with an acid. In some embodiments, treating the compound of formula 21 with ammonia is performed in a solvent mixture comprising methanol and tetrahydrofuran.

In some embodiments, the process for preparing Compound 1 further includes recrystallizing Compound 1 from a suitable solvent. In some embodiments, the suitable solvent includes MeOH, THF, water, or a combination thereof. In other embodiments, the suitable solvent includes acetone and water.

In some embodiments, the process for preparing Compound 1 comprises transforming a compound of formula 16:

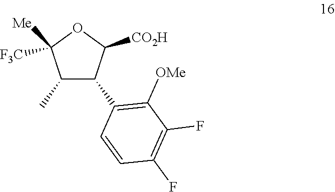 [SMILES: COc1c([C@H]2[C@H](C(=O)O)O[C@@](C)(C(F)(F)F)[C@H]2C)ccc(F)c1F]

to the compound of formula 17, which may also be referred to as an (R)-AMB or an (R)-α-methylbenzylamine salt of the compound of formula 16.

In some embodiments, the compound of formula 17 may be prepared directly from the compound of formula 16 by contacting the compound of formula 16 with (R)-α-methylbenzylamine. In some embodiments, the compound of formula 17 may be prepared directly from the compound of formula 16 by contacting the compound of formula 16 with (R)-α-methylbenzylamine in an appropriate solvent. In some embodiments, the compound of formula 17 may be prepared directly from the compound of formula 16 by dissolving the compound of formula 16 and (R)-α-methylbenzylamine in an appropriate solvent. One of ordinary skill will appreciate that there are many solvents compatible with transforming the compound of formula 16 to the compound of formula 17. In some embodiments, the appropriate solvent is toluene.

In some embodiments, the process for preparing Compound 1 further includes transforming a compound of formula 15:

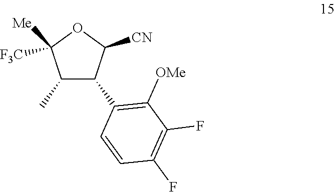 [SMILES: COc1c([C@H]2[C@H](C#N)O[C@@](C)(C(F)(F)F)[C@H]2C)ccc(F)c1F]

to the compound of formula 16.

In some embodiments, transforming the compound of formula 15 to the compound of formula 16 includes hydrolyzing the CN group of the compound of formula 15. Any base or acid suitable for hydrolyzing the CN group without affecting other functional groups in the compound of formula 16 may be used. In some embodiments, a strong base (such as NaOH, KOH, and the like) or strong acid (HCl, sulfuric acid, or the like) may be used. In other embodiments, the CN group in the compound of formula 15 is enzymatically hydrolyzed using a nitrilase. The CN hydrolysis of the compound of formula 15 may be conducted in a solvent or solvent mixture. For example, ethanol, methanol, 1-propanol, 2-propanol, dioxane, water, THF, or a mixture thereof may be used. The hydrolysis reaction may be conducted at about 25-75° C., about 30-70° C., about 35-65° C., about 40-60° C., about 45-60° C., about 50-60° C., or about 55° C. As used in this paragraph, the term “about” in front of a temperature range applies to both ends of the range. It also means±2.5° C. In some embodiments, transforming a compound of formula 15 to the compound of formula 16 includes treating the compound of formula 15 with a base. One of ordinary skill will appreciate that many bases would be compatible with the process of transforming the compound of formula 15 to the compound of formula 16. In some embodiments, the base is potassium hydroxide.

In some embodiments, the process for preparing Compound 1 further includes transforming a compound of formula 14:

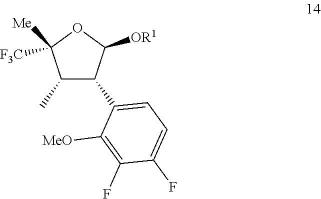

to the compound of formula 15,
where:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.

In some embodiments, the compound of formula 14 is a compound of formula 14′:

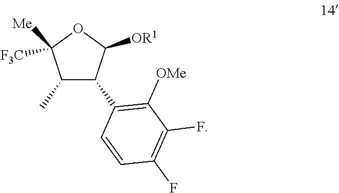

In some embodiments, the compound of formula 14 is a compound of formula 14″:

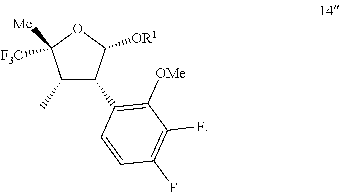

In some embodiments, R¹ is —C(O)—Z. In some embodiments, Z is methyl or 4-nitrophenyl. In some embodiments, Z is 4-nitrophenyl.

In some embodiments, the compound of formula 14 is selected from:

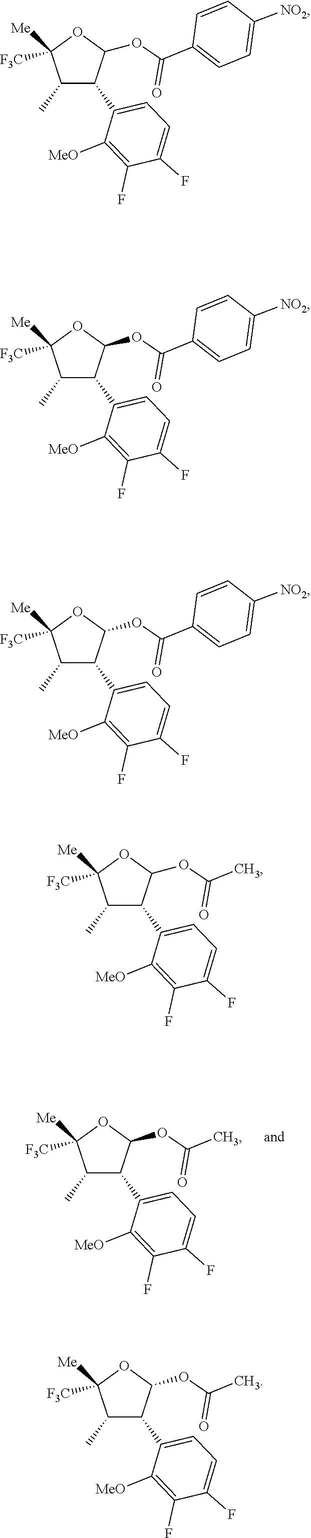

In some embodiments, the compound of formula 14 is a compound of formula 14-A:

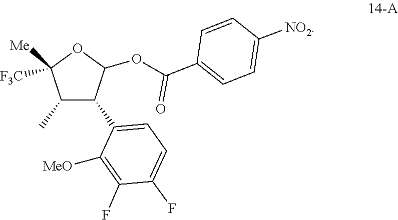 [SMILES: COc1c([C@H]2C(OC(=O)c3ccc([N+](=O)[O-])cc3)O[C@@](C)(C(F)(F)F)[C@H]2C)ccc(F)c1F]

In some embodiments, the compound of formula 14 is a compound of formula 14-B:

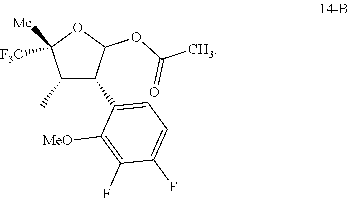 [SMILES: COc1c([C@H]2C(OC(C)=O)O[C@@](C)(C(F)(F)F)[C@H]2C)ccc(F)c1F]

In some embodiments, the compound of formula 14 is a compound of formula 14′-A:

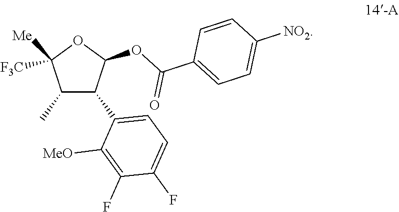 [SMILES: COc1c([C@H]2[C@H](OC(=O)c3ccc([N+](=O)[O-])cc3)O[C@@](C)(C(F)(F)F)[C@H]2C)ccc(F)c1F]

In some embodiments, the compound of formula 14 is a compound of formula 14′-B:

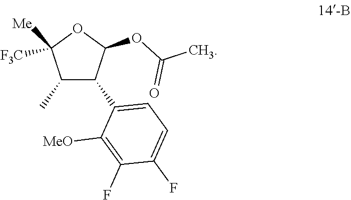 [SMILES: COc1c([C@H]2[C@H](OC(C)=O)O[C@@](C)(C(F)(F)F)[C@H]2C)ccc(F)c1F]

In some embodiments, transforming a compound of formula 14 to the compound of formula 15 includes treating the compound of formula 14 with a cyanating reagent. In some embodiments, the cyanating agent is selected from the group consisting of trimethylsilyl cyanide, diethylaluminum cyanide, KCN, NaCN, TBACN, and HCN. In some embodiments, the cyanating reagent is trimethylsilyl cyanide. In some embodiments, the compound of formula 14 is treated with 1.35-1.65 equivalents of trimethylsilyl cyanide. In some embodiments, treating the compound of formula 14 with a cyanating reagent is performed in the presence of a Lewis acid. In some embodiments, the Lewis acid is selected from boron trifluoride diethyl etherate (BF3·OEt2), TiCl₄, InCl₃, AgSbF₆, iodine, ZnBr₂, Al(OiPr)₃, MgCl₂, Mn(acac)₂, MnCl₂, TMSOTf, and SnCl₄. In some embodiments, the Lewis acid is BF₃·OEt₂. In some embodiments, 0.9-1.1 equivalents of the BF₃0Et₃ are present, based on the compound of formula 14. The cyanation reaction may be conducted in an organic solvent, for example toluene, dichloromethane, 2-methyl THF, acetonitrile, methanol, 1,2-dichloroethane, nitromethane, CPME, MTBE, DMAc, t-BuOAc, and the like. In some embodiments, the cyanation reaction is conducted at a temperature between −28 and 0° C., or between −28 and −12° C. In some embodiments, the cyanation reaction is quenched with acetone (e.g., about 1.0 equivalents of acetone). In some embodiments, the quench is conducted at a temperature between −28 and 0° C., or between −28 and −12° C. In some embodiments, the compound of formula 15 is obtained by partitioning the reaction mixture between an organic layer comprising ethanol and an aqueous layer comprising aqueous potassium hydroxide.

Other non-limiting examples of compounds of formula 14 include:

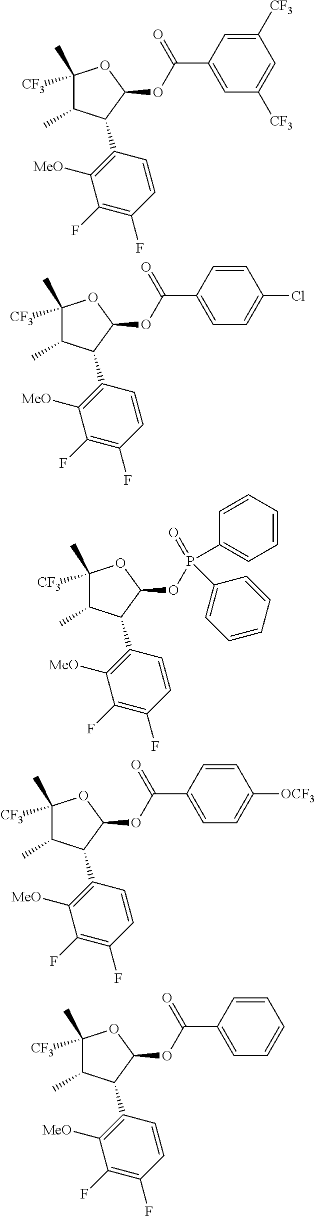

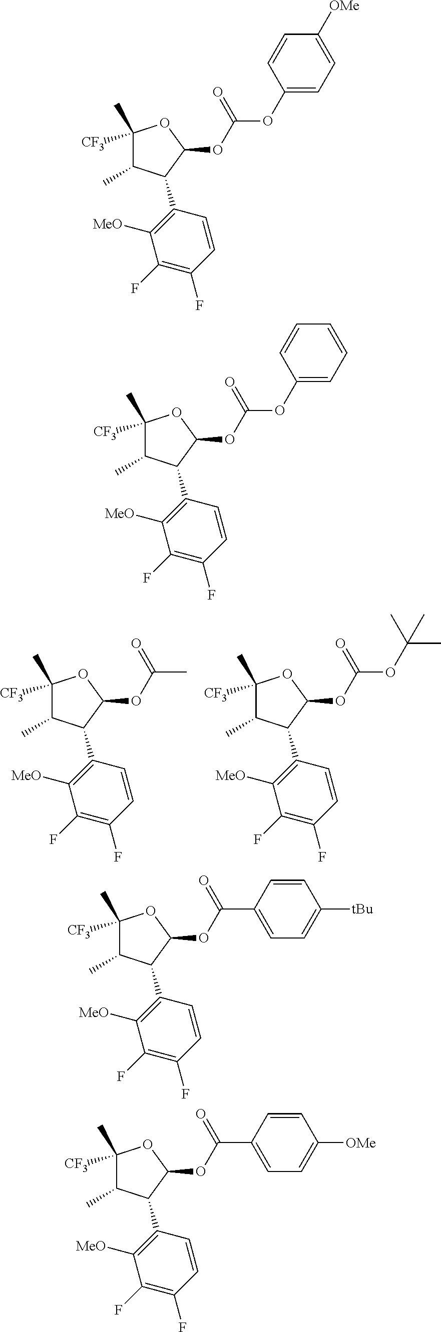

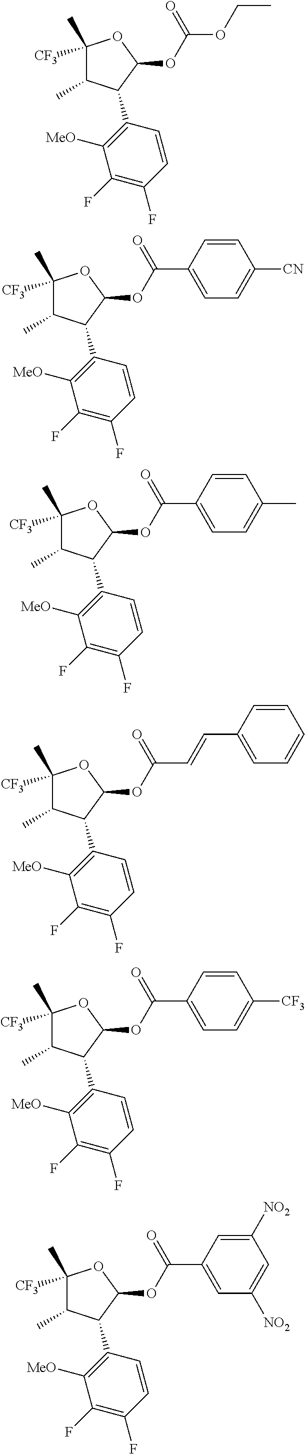

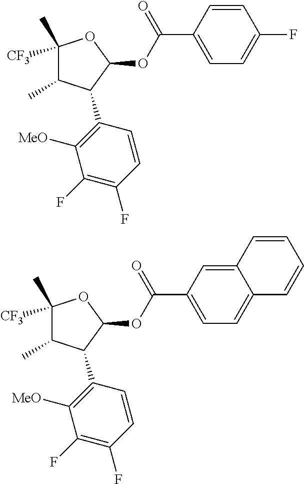 [SMILES: COc1c([C@H]2[C@H](OC(=O)c3ccc(F)cc3)O[C@@](C)(C(F)(F)F)[C@H]2C)ccc(F)c1F ; COc1c([C@H]2[C@H](OC(=O)c3ccc4ccccc4c3)O[C@@](C)(C(F)(F)F)[C@H]2C)ccc(F)c1F]

In some embodiments, the process for preparing Compound 1 further includes transforming a compound of formula 13:

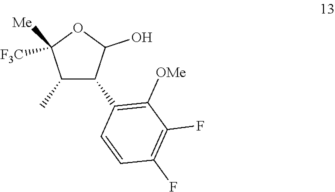 [SMILES: COc1c([C@H]2C(O)O[C@@](C)(C(F)(F)F)[C@H]2C)ccc(F)c1F]

to the compound of formula 14.

In some embodiments, the compound of formula 13 has the formula 13′:

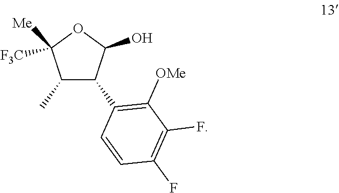 [SMILES: COc1c([C@H]2[C@H](O)O[C@@](C)(C(F)(F)F)[C@H]2C)ccc(F)c1F]

In some embodiments, the compound of formula 13 has the formula 13″:

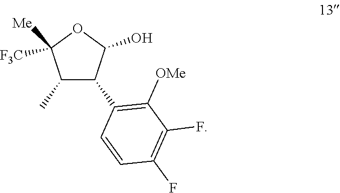 [SMILES: COc1c([C@H]2[C@@H](O)O[C@@](C)(C(F)(F)F)[C@H]2C)ccc(F)c1F]

In some embodiments, transforming a compound of formula 13 to the compound of formula 14 includes contacting the compound of formula 13 with an acid anhydride or an acid halide to afford the compound of formula 14. In some embodiments, contacting the compound of formula 13 with an acid anhydride or an acid halide is performed in the presence of a base and a catalyst. One of ordinary skill in the art will appreciate that there are many bases that would be compatible with the process of transforming the compound of formula 13 to the compound of formula 14. In some embodiments, the base is a non-nucleophilic base. In some embodiments, the base is selected from TEA, pyridine, Hunig's base, K₂CO₃, Na₂CO₃, NaHCO₃, 2,6-Lutidine, NMM, and DABCO. In some embodiments, the base is triethylamine and the catalyst is 4-dimethylaminopyridine (DMAP). In some embodiments, the acid anhydride is acetic anhydride. In some embodiments, the acid halide is 4-nitrobenzoyl chloride. In some embodiments, the transformation of the compound of formula 13 to the compound of formula 14 is performed in a polar solvent. In some embodiments, the polar solvent is selected from toluene, cyclopentyl methyl ether (CPME), dichloromethane, dichloroethane, pyridine, chloroform, acetonitrile, THF, 2-MeTHF, EtOAc, IPAC or combinations thereof.

In some embodiments, the process for preparing Compound 1 further includes recrystallizing the compound of formula 14 from a suitable solvent. In some embodiments, the suitable solvent includes acetone, water, and combinations thereof.

In some embodiments, the process for preparing Compound 1 further includes transforming a compound of formula 12:

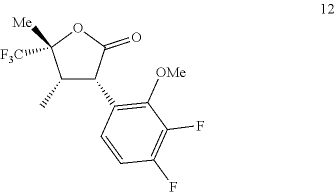 [SMILES: COc1c([C@H]2C(=O)O[C@@](C)(C(F)(F)F)[C@H]2C)ccc(F)c1F]

to the compound of formula 13.

In some embodiments, transforming the compound of formula 12 to the compound of formula 13 includes treating the compound of formula 12 with a reducing reagent. In some embodiments, the reducing reagent is selected from diisobutylaluminum hydride, Red-Al, NaBH₄/BF₃, titanocene with polymethylhydrosiloxane and phenylsilane. In some embodiments, the reducing reagent is diisobutylaluminum hydride. The reduction reaction may be conducted in an organic solvent or solvent mixture. Suitable solvents include toluene, dichloromethane, 2-methyl THF, THF, TFT, MTBE, CPME, heptane, or a mixture thereof. The reaction may be conducted at below room temperature, for example, about −78° C. to 0° C., about −60° C. to 0° C., about −50° C. to −10° C., about −40° C. to −10° C., about −30° C. to −10° C., about −30° C. to −15° C., about −25° C. to −15° C., or about −20° C. The reduction reaction may be conducted in the presence of CuCl, CuI, CuTol, CuBr, CuF, Cu(II)Cl₂, DMAP, 2,6-lutidine, LiI, or pyridine.

In some embodiments, the process for preparing Compound 1 further includes transforming a compound of formula 11:

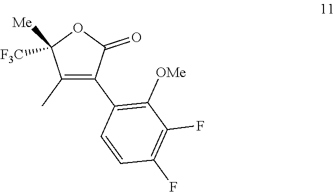 [SMILES: COc1c(C2=C(C)[C@](C)(C(F)(F)F)OC2=O)ccc(F)c1F]

to the compound of formula 12.

In some embodiments, transforming a compound of formula 11 to the compound of formula 12 comprises a hydrogenation of the compound of formula 11. In some embodiments, the hydrogenation is performed in the presence of a hydrogenation catalyst. In some embodiments, the hydrogenation catalyst is selected from Pd/C, Pd/Al₂O₃, Pt/C, Ni (Raney), Co (Raney), Rh/C, Ir/C, Ru/C, Pd(OH)₂, homogeneous chiral Ru and Rh. In some embodiments, the hydrogenation is performed in the presence of a suitable hydrogen source. In some embodiments, the hydrogen source is selected from H₂ gas, NiCl₂/NaBH₄ in methanol, and Et₃SiH. In some embodiments, the hydrogenation is conducted in the presence of H₂ gas using Pd/C (e.g., Johnson Matthey Catalog No. A503032-5 or A503014-5) as a catalyst. The hydrogenation reaction may be conducted in an organic solvent at between about 20 to 40 bar. In some embodiments, the hydrogenation reaction also may be conducted in an organic solvent at between about 5 to 40 barg. In some embodiments, the hydrogenation reaction may be performed at a temperature of 10-50° C. A lower pressure may be use with high temperature and vice versa. For instance, about 5 bar may be suitable at about 40° C. Conversely, about 15-20 bar may be suitable at about 30° C. The skilled artisan can match pressure, temperature and reaction time to obtain desirable results. The hydrogenation reaction may be conducted in an organic solvent or a solvent mixture. In one embodiment, the organic solvent is IPA, EtOAc, MeOH, nBuOH, THF, MTBE, CPME, IPAc, nBuAc, Toluene, Ethanol, or a mixture thereof. In some embodiments, the hydrogenation is performed in a solvent mixture comprising 2-propanol, tetrahydrofuran, and catalytic trifluoroacetic acid. The asymmetric hydrogenation reaction may be conducted in the presence of citric acid, benzoic acid, TFA, AcOH, H₂SO₄, H₃PO₄, MSA, Cs₂CO₃, CuCl, MgF₂, LiBr, CsF, ZnI, LiOTf, imidazole, KF, Bu₄NOAc, and/or NH₄BF₄.

In another aspect, the disclosure relates to a compound selected from:

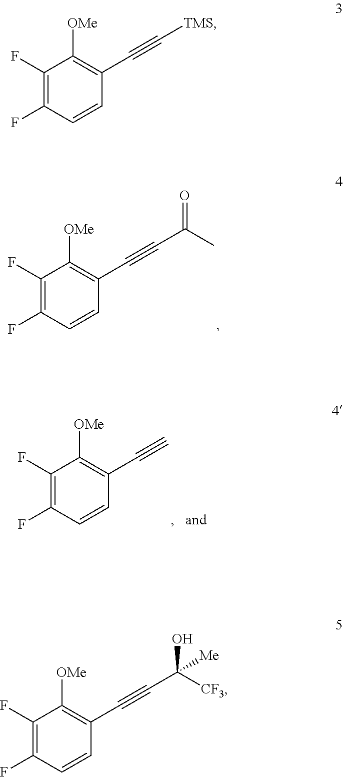 [SMILES: COc1c(C#C[Si](C)(C)C)ccc(F)c1F ; COc1c(C#CC(C)=O)ccc(F)c1F ; C#Cc1ccc(F)c(F)c1OC ; COc1c(C#C[C@@](C)(O)C(F)(F)F)ccc(F)c1F]

or a salt thereof

### Enumerated Embodiments

In some embodiments, the disclosure relates to:
1. A method of treating chronic pain in a subject, comprising administering to the subject Compound 1:

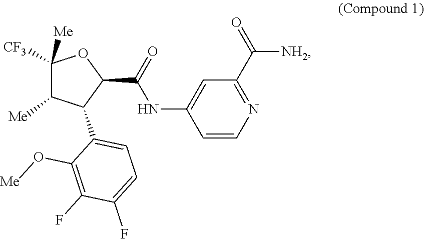 [SMILES: COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof, in an amount of about 15 to about 80 mg per day.

2. The method of embodiment 1, wherein the chronic pain is lessened in the subject.

3. The method of embodiment 1 or 2, wherein Compound 1, or a pharmaceutically acceptable salt thereof, is administered in an amount of about 20 to about 75 mg per day, or about 20to about 30 mg per day, or about 40 to about 50 mg per day, or about 60 to about 70 mg per day.

4. The method of any one of embodiments 1-3, wherein Compound 1, or a pharmaceutically acceptable salt thereof, is administered in an amount of about 20 mg per day, or about 23 mg per day, or about 25 mg per day, or about 45 mg per day, or about 46 mg per day, or about 50 mg per day, or about 65 mg per day, or about 69 mg per day, or about 70 mg per day.

5. The method of any one of embodiments 1-4, wherein Compound 1, or a pharmaceutically acceptable salt thereof, is administered in an amount of 45 mg per day.

6. The method of any one of embodiments 1-4, wherein Compound 1, or a pharmaceutically acceptable salt thereof, is administered in an amount of 50 mg per day.

7. The method of any one of embodiments 1-4, wherein Compound 1, or a pharmaceutically acceptable salt thereof, is administered in an amount of 70 mg per day.

8. The method of any one of embodiments 1-7, wherein the chronic pain is moderate to severe chronic pain.

9. The method of embodiment 8, wherein the subject has a baseline weekly average pain score of ≥4 on an 11-point Numeric Pain Rating Scale (NPRS).

10. The method of any one of embodiments 1-9, wherein the chronic pain is neuropathic pain, musculoskeletal pain, or visceral pain.

11. The method of any one of embodiments 1-10, wherein the chronic pain is diabetic peripheral neuropathy.

12. The method of any one of embodiments 1-11, wherein the subject experiences a change of −1 to −4 in weekly average of daily pain on an 11-point Numeric Pain Rating Scale (NPRS) at week 12 of treatment, relative to baseline.

13. The method of embodiment 12, wherein the subject experiences a change of −2.0 to −2.5 in weekly average of daily pain on an 11-point Numeric Pain Rating Scale (NPRS) at week 12 of treatment, relative to baseline.

14. The method of any one of embodiments 1-13, wherein the subject experiences an improvement in sleep, as determined by a change in Daily Sleep Interference Scale (DSIS) score at week 12 of treatment, relative to baseline.

15. A method of treating acute pain in a subject, comprising administering to the subject Compound 1:

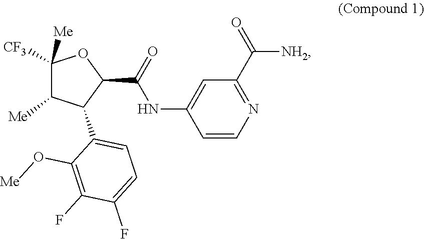 [SMILES: COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof, in a first dose of about 85 to about 115 mg, followed by subsequent doses of about 40 to about 60 mg every 12 hours.

16. The method of embodiment 15, wherein the acute pain is lessened in the subject.

17. The method of embodiment 15 or 16, wherein the first dose is about 100 mg, and the subsequent doses are about 50 mg.

18. The method of any one of embodiments 15-17, wherein the first dose is 100 mg, and the subsequent doses are 50 mg.

19. The method of any one of embodiments 15-18, wherein the acute pain is moderate to severe acute pain.

20. The method of embodiment 19, wherein the subject has a baseline pain score of ≥4 on an 11-point Numeric Pain Rating Scale (NPRS) or where the subject has a baseline pain level of moderate or severe on a Verbal Categorical Rating Scale.

21. The method of any one of embodiments 15-20, wherein the acute pain is acute post-operative pain or postsurgical pain.

22. The method of any one of embodiments 15-21, wherein the acute pain is bunionectomy pain.

23. The method of any one of embodiments 15-21, wherein the acute pain is abdominoplasty pain.

24. The method of any one of embodiments 15-23, wherein the subject experiences a time-weighted sum of pain intensity difference, relative to baseline, as recorded on an 11-point Numeric Pain Rating Scale (NPRS), from 0 to 48 hours (SPID48) of 75.0 to 125.0.

25. The method of embodiment 24, wherein the subject experiences a time-weighted sum of pain intensity difference, relative to baseline, as recorded on an 11-point Numeric Pain Rating Scale (NPRS), from 0 to 48 hours (SPID48) of 95.0 to 105.0.

26. The method of embodiment 25, wherein the subject experiences a time-weighted sum of pain intensity difference, relative to baseline, as recorded on an 11-point Numeric Pain Rating Scale (NPRS), from 0 to 48 hours (SPID48) of about 99.9.

27. The method of any one of embodiments 15-26, wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0.

28. The method of embodiment 27, wherein the subject experiences a difference from placebo in time-weighted sum of pain intensity difference, relative to baseline, as recorded on an 11-point Numeric Pain Rating Scale (NPRS), from 0 to 48 hours (SPID48) of 25.0 to 35.0.

29. The method of embodiment 28, wherein the subject experiences a difference from placebo in time-weighted sum of pain intensity difference, relative to baseline, as recorded on an 11-point Numeric Pain Rating Scale (NPRS), from 0 to 48 hours (SPID48) of about 29.3.

30. The method of any one of embodiments 15-29, wherein the subject experiences a change of −2.5 to −4.5 in pain intensity on an 11-point Numeric Pain Rating Scale (NPRS) after 48 hours, relative to baseline.

31. The method of embodiment 30, wherein the subject experiences a change of −3.0 to −4.0 in pain intensity on an 11-point Numeric Pain Rating Scale (NPRS) after 48 hours, relative to baseline.

32. The method of embodiment 30, wherein the subject experiences a change of about −3.4 in pain intensity on an 11-point Numeric Pain Rating Scale (NPRS) after 48 hours, relative to baseline.

33. The method of any one of embodiments 15-32, wherein the subject experiences a 51% reduction in pain intensity on an 11-point Numeric Pain Rating Scale (NPRS) after 48 hours, relative to baseline.

34. The method of any one of embodiments 15-33, wherein the subject experiences a 2-point reduction in pain intensity, relative to baseline, as recorded on an 11-point Numeric Pain Rating Scale (NPRS), 115 to 480 minutes after administration of the first dose.

35. The method of embodiment 34, wherein the subject experiences a 2-point reduction in pain intensity, relative to baseline, as recorded on an 11-point Numeric Pain Rating Scale (NPRS), 210 to 270 minutes after administration of the first dose.

36. The method of embodiment 35, wherein the subject experiences a 2-point reduction in pain intensity, relative to baseline, as recorded on an 11-point Numeric Pain Rating Scale (NPRS), about 240 minutes after administration of the first dose.

37. The method of any one of embodiments 15-23, wherein the subject experiences a time-weighted sum of pain intensity difference, relative to baseline, as recorded on an 11-point Numeric Pain Rating Scale (NPRS), from 0 to 48 hours (SPID48) of 100.0 to 140.0.

38. The method of embodiment 37, wherein the subject experiences a time-weighted sum of pain intensity difference, relative to baseline, as recorded on an 11-point Numeric Pain Rating Scale (NPRS), from 0 to 48 hours (SPID48) of 113.0 to 123.0.

39. The method of embodiment 38, wherein the subject experiences a time-weighted sum of pain intensity difference, relative to baseline, as recorded on an 11-point Numeric Pain Rating Scale (NPRS), from 0 to 48 hours (SPID48) of about 118.4.

40. The method of any one of embodiments 15-23 and 37-39, wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0.

41. The method of embodiment 40, wherein the subject experiences a difference from placebo in time-weighted sum of pain intensity difference, relative to baseline, as recorded on an 11-point Numeric Pain Rating Scale (NPRS), from 0 to 48 hours (SPID48) of 43.0 to 53.0.

42. The method of embodiment 41, wherein the subject experiences a difference from placebo in time-weighted sum of pain intensity difference, relative to baseline, as recorded on an 11-point Numeric Pain Rating Scale (NPRS), from 0 to 48 hours (SPID48) of about 48.4.

43. The method of any one of embodiments 15-23 and 37-42, wherein the subject experiences a change of −2.5 to −4.5 in pain intensity on an 11-point Numeric Pain Rating Scale (NPRS) after 48 hours, relative to baseline.

44. The method of embodiment 43, wherein the subject experiences a change of −3.0 to −4.0 in pain intensity on an 11-point Numeric Pain Rating Scale (NPRS) after 48 hours, relative to baseline.

45. The method of embodiment 44, wherein the subject experiences a change of about −3.4 in pain intensity on an 11-point Numeric Pain Rating Scale (NPRS) after 48 hours, relative to baseline.

46. The method of any one of embodiments 15-23 and 37-45, wherein the subject experiences a 47% reduction in pain intensity on an 11-point Numeric Pain Rating Scale (NPRS) after 48 hours, relative to baseline.

47. The method of any one of embodiments 15-23 and 37-46, wherein the subject experiences a 2-point reduction in pain intensity, relative to baseline, as recorded on an 11-point Numeric Pain Rating Scale (NPRS), 90 to 180 minutes after administration of the first dose.

48. The method of embodiment 47, wherein the subject experiences a 2-point reduction in pain intensity, relative to baseline, as recorded on an 11-point Numeric Pain Rating Scale (NPRS), 100 to 140 minutes after administration of the first dose.

49. The method of embodiment 48, wherein the subject experiences a 2-point reduction in pain intensity, relative to baseline, as recorded on an 11-point Numeric Pain Rating Scale (NPRS), about 119 minutes after administration of the first dose.

50. The method of any one of embodiments 15-49, wherein Compound 1 is administered in a pharmaceutical composition comprising: 45-55 wt % of a solid dispersion comprising a polymer and Compound 1; 42-50 wt % of microcrystalline cellulose; 2-4 wt % of croscarmellose sodium; and 0.5-1.5 wt % of magnesium stearate.

51. The method of embodiment 50, wherein the pharmaceutical composition is a tablet core composition.

52. The method of embodiment 50, wherein the tablet core composition is coated with a tablet coating.

53. The method of any one of embodiments 15-52, wherein Compound 1 is administered in a pharmaceutical composition comprising about 50 mg of Compound 1.

54. The method of any one of embodiments 15-53, wherein Compound 1 is administered in a pharmaceutical composition comprising about 184 mg of microcrystalline cellulose.

55. The method of any one of embodiments 15-54, wherein Compound 1 is administered in a pharmaceutical composition comprising about 12 mg of croscarmellose sodium.

56. The method of any one of embodiments 15-55, wherein Compound 1 is administered in a pharmaceutical composition comprising about 4 mg of magnesium stearate.

57. The method of any one of embodiments 15-56, wherein Compound 1 is administered in a pharmaceutical composition comprising about 200 mg of a solid dispersion comprising a polymer and Compound 1.

58. The method of any one of embodiments 15-57, wherein Compound 1 is administered in a pharmaceutical composition comprising an intragranular blend and extragranular blend.

59. The method of embodiment 58, wherein the intragranular blend comprises 0.4 mg of magnesium stearate, and the extragranular blend comprises 3.6 mg of magnesium stearate.

### EXAMPLES

### Abbreviations

Unless otherwise noted, or where the context dictates otherwise, the following abbreviations shall be understood to have the following meanings:

| Abbreviation | Definition |
|---|---|
| TPGS | Tocopherol polyethylene glycol succinate |
| HB/APAP | Hydrocodone bitartrate/acetaminophen |
| NPRS | Numeric Pain Rating Scale |
| VRS | Verbal Categorical Rating Scale |
| qd | Once per day |
| tid | Three times per day |
| q24 h | Every 24 hours |
| q12 h | Every 12 hours |
| q6 h | Every 6 hours |
| n | Number of subjects meeting a specified criterion |
| n (%) | Number of subjects meeting a specified criterion |
| | (percentage of the total number of subjects in |
| | study arm meeting that criterion) |
| SPID24 | NPRS pain intensity difference, relative to |
| | baseline, from 0 to 24 hours after the first dose |
| | for a given study arm |
| SD | Standard deviation |
| SE | Standard error |
| CI | Confidence interval |
| SPID22 | NPRS pain intensity difference, relative to |
| | baseline, from 2 to 24 hours after the first dose |
| | for a given study arm |
| SPID48 | NPRS pain intensity difference, relative to |
| | baseline, from 0 to 48 hours after the first dose |
| | for a given study arm |
| Cₘₐₓ | Maximum observed concentration |
| AUC_{τ} | Area under the curve over a dosing interval (τ) |
| TEAE | Treatment-emergent adverse event |

### Example 1

### Preparation of (2R,3S,4S,5R)-4-[[3-(3,4-difluoro-2-methoxy-phenyl)-4,5-dimethyl-5-(trifluoromethyl)tetrahydrofuran-2-carbonyl]amino]pyridine-2-carboxamide (Compound 1)

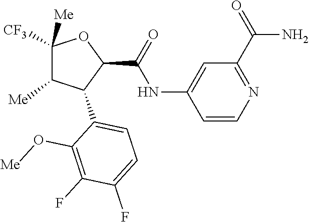 [SMILES: COc1c([C@H]2[C@H](C(=O)Nc3ccnc(C(N)=O)c3)O[C@@](C)(C(F)(F)F)[C@H]2C)ccc(F)c1F]

Compound 1 can be prepared by methods known in the art, including the methods described in International Publication No. WO 2021/113627 (Example 3), International Publication No. WO 2022/256660, and International Publication No. WO 2024/123815, each of which is incorporated by reference.

### Example 2

### SDD Tablet Process

### Step 1: Preparation of Compound 1 Spray-Dried Dispersion (SDD)

About 2466.7 gm of dichloromethane and about 2466.7 gm of methanol were combined in a glass vessel. To the resulting mixture was added about 100 gm of Compound 1 at room temperature. Upon complete dissolution of Compound 1, about 300 gm of HPMCAS was added to the mixture at room temperature. Upon complete dissolution of the HPMCAS, the resulting mixture was spray-dried using a MicraSpray (MS-35) spray dryer with a 0.8 mm two fluid nozzle, 3 mm spacer, 2.6 mm air cap, 150 mesh PTFE inline filter, and a collection container attached to the cycle and pulseback filters. The process parameters of the MS-35 spray dryer are summarized in the following table:

Spray Dryer Process Parameters

| Parameter | Target | (+/−) Range |
|---|---|---|
| Outlet Temp (° C.) | 48.0 | 45.0-51.0 |
| Process Gas Flow (kg/hr) | 35.0 | 33-37 |
| Nozzle Gas Flow (kg/hr) | 4.5 | 4.2-4.8 |
| Solution Feed Rate (kg/hr) | | |
| Solution Feed Rate (gm/min) | 30.0 | 27-33 |
| Estimated Spray time (hr) | 3.0 | |

To begin spray drying, the MS-35 was preheated, and when the target outlet temperature of 48° C. was reached an equilibrium solution was sprayed until all the parameters were stable and within the target range. Once all the parameters have stabilized and are within the target range the MS-35 spray dryer began spraying the solution containing Compound 1 and the polymer. The process parameters may be adjusted during the run to maintain it within the working range.

The wet SDD was transferred to appropriate size trays. Each tray was filled to a depth of about 1 inch of powder and placed in a vacuum oven at 40° C. with nitrogen purging. After 12-72 hours samples were pulled from the trays to check for residual solvent levels. Once the solvent levels were below the specifications (dichloromethane (<600 ppm) and methanol (<3000 ppm)) all powder from the trays were combined.

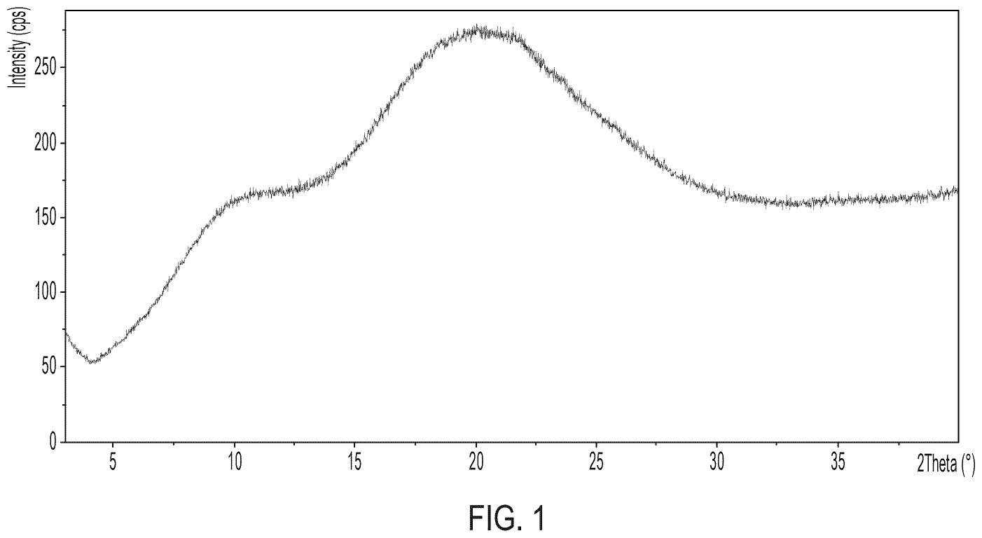

The SDD was transferred to a secondary drying chamber for further drying. Upon completion of drying, X-ray powder diffraction (XRPD) analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40° 2θ with a step size of 0.0131° and 48.195 s per step. The obtained XRPD diffractogram is depicted in FIG. 1.

The composition of the SDD is summarized in Table 1:

TABLE 1
Composition of Compound 1 Spray-Dried Dispersion

| Component | Content (% w/w) |
|---|---|
| Compound 1 | 25.0 |
| Hydroxypropyl Methylcellulose | 75.0 |
| Acetate Succinate (HPMCAS) | |
| Total | 100.0 |

### Step 2: Preparation of Tablet Comprising Compound 1 SDD

Tablets having the composition set forth in Table 6 were prepared. Compound 1 SDD from Step 1, microcrystalline cellulose, lactose monohydrate, and croscarmellose sodium were each passed through a 20 mesh screen and combined in a 10 L Bohle blender. The mixture was blended for about 2.5 minutes at 32 rpm. Sodium stearyl fumarate was passed through a 60 mesh screen and added to the blended mixture and further blended for about 1.5 minutes at 32 rpm.

The resulting blended mixture was dry granulated using a GERTEIS® roller compactor and an in-line mill. The resulting milled granules were added to a 5 L Bohle blender along with microcrystalline cellulose and croscarmellose sodium that was passed through a 20 mesh screen. The resulting mixture was blended for 8.5 minutes at 32 rpm. After blending, sodium stearyl fumarate that was passed through a 60 mesh screen was added to the blended mixture and blended for an additional 2 minutes at 32 rpm. Process parameters for the roller compactor and in-line mill are summarized in Table 2:

TABLE 2
Process Parameters

| Parameter | Target | (+/−) Range |
|---|---|---|
| Roll Gap (mm) | 2.0 | 1.0-3.0 |
| Roll Pressure (kN/cm) | 9 | 6-12 |
| Roll Speed (rpm) | 2 | 1.5-2.5 |
| Gap Control | On | |
| Agitator speed (rpm) | 15 | |
| Granulation Speed cw/ccw (rpm) | 80/80 | 60-100/60-100 |
| Oscillation (cw/ccw) (deg) | 330-360 | 320-340/350-370 |
| PID “P value” | 3 | NA |
| PID “I value” | 3000 | 2000-7000 |
| PID “D value” | 0 | NA |
| Torque Control | off | |
| Tamp/Feed Ratio (%) | 165 | 110-500 |
| Feed Factor | 0.3 | 0.1-0.9 |
| Roll type | Smooth/Smooth | |
| Screen Size (mm) | 1.00 | |
| Rotor type | Pocketed rotor | |

The resulting mixture was then compressed into tablets using a PICCOLA® tablet press to yield tablets with each containing 10 mg of Compound 1. The press had an 8 mm standard round concave tooling and paddle feeder. The turret speed was set to 30 rpm and the paddle speed was set to 25 rpm. The target weight and hardness of the tablets is summarized in Table 3:

TABLE 3

| | Weight (mg) | Hardness (kP) |
|---|---|---|
| Target | 200 | 8.00 |
| Acceptance Limits | 180-220 | 6.0-10.0 |

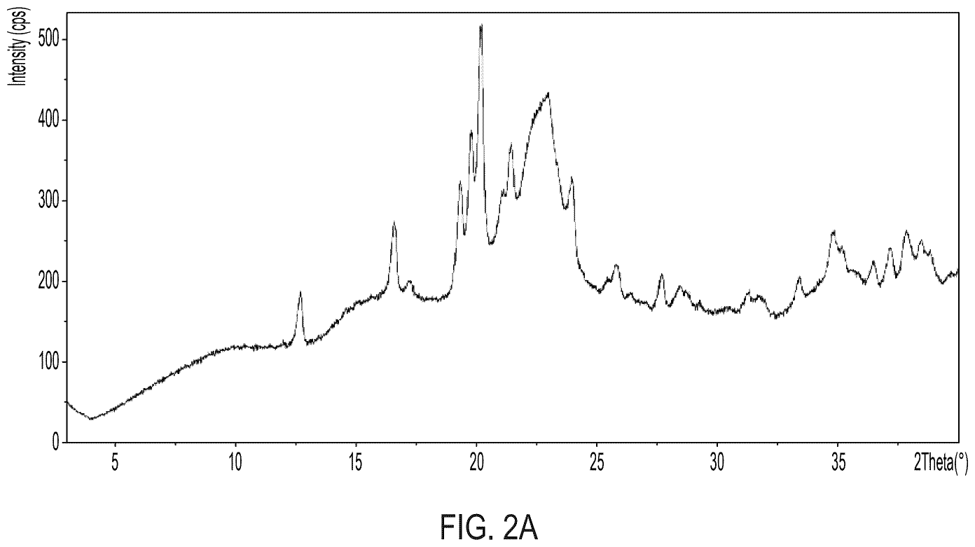

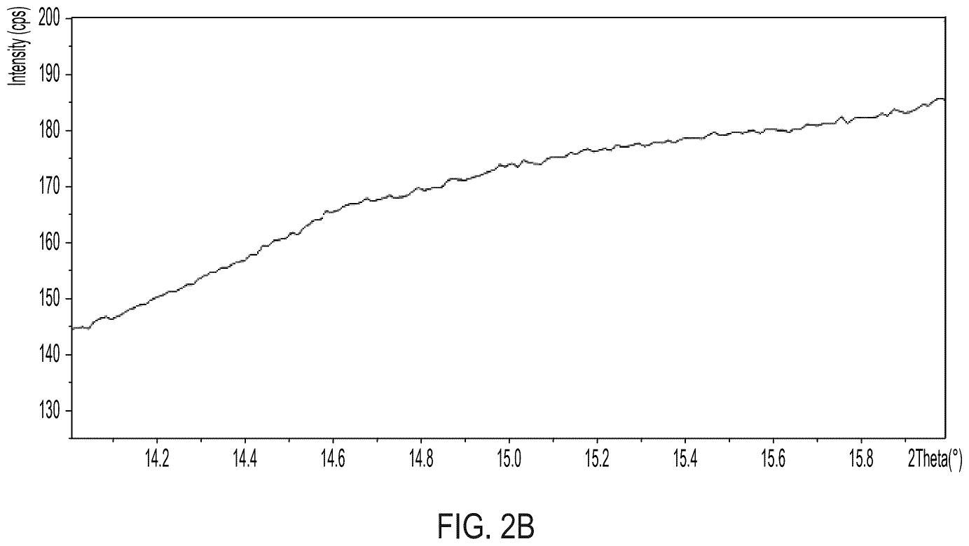

The resulting tablets were analyzed by X-ray powder diffraction (XRPD) and solid state nuclear magnetic resonance (ssNMR) after being ground into a uniform powder. XRPD analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40°2θ with a step size of 0.0131° and 48.195 s per step, and scanned over the range of about 14° to about 16°2θ with a step size of 0.0131° and 1497.870 s per step. The obtained XRPD diffractogram is depicted in FIGS. 2A and 2B.

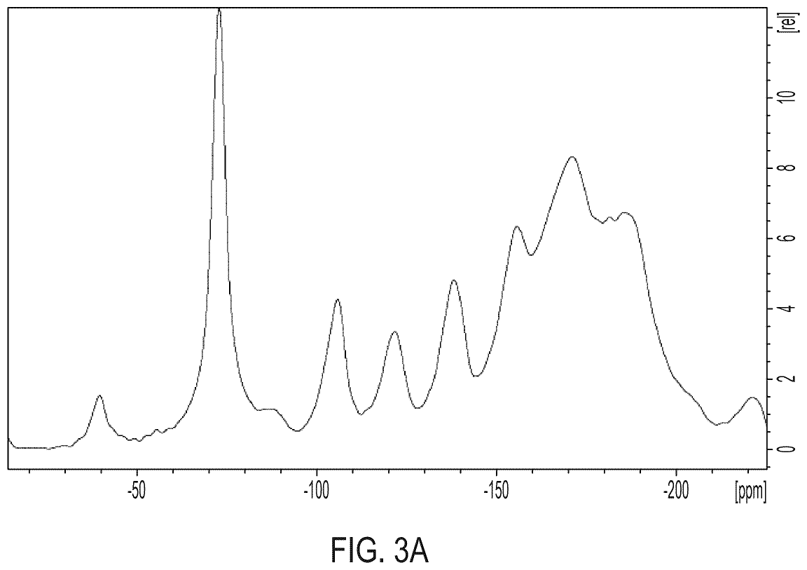

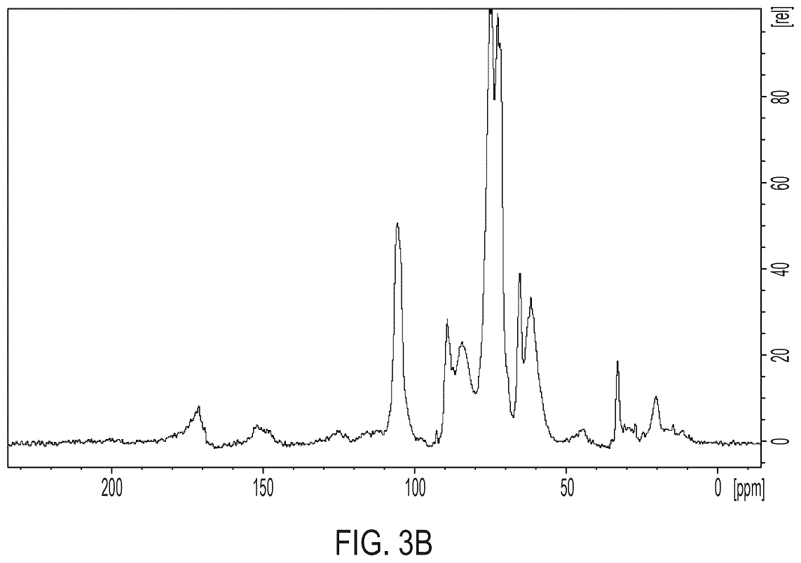

Solid state NMR analysis was conducted on a Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz. The obtained fluorine and carbon NMR spectra are depicted in FIGS. 3A and 3B, respectively, and the peak lists appear in Tables 4 and 5.

TABLE 4
¹⁹F MAS peak list:

| Peak # | δ [ppm] | Intensity |
|---|---|---|
| 1 | −40.1 | 1.51E0 |
| 2 | −73.2 | 1.23E1 |
| 3 | −105.9 | 4.26E0 |
| 4 | −121.8 | 3.33E0 |
| 5 | −137.9 | 4.80E0 |
| 6 | −155.6 | 6.33E0 |
| 7 | −171.1 | 8.30E0 |
| 8 | −220.6 | 1.49E0 |

TABLE 5
¹³C CPMAS peak list:

| Peak # | δ [ppm] | Intensity |
|---|---|---|
| 1 | 170.6 | 8.85E0 |
| 2 | 151.5 | 4.48E0 |
| 3 | 125.2 | 3.23E0 |
| 4 | 92.6 | 3.24E0 |
| 5 | 89.2 | 2.87E1 |
| 6 | 84.3 | 2.33E1 |
| 7 | 75.1 | 9.99E1 |
| 8 | 72.7 | 9.87E1 |
| 9 | 65.3 | 3.92E1 |
| 10 | 61.7 | 3.37E1 |
| 11 | 44.5 | 3.52E0 |
| 12 | 33.2 | 1.92E1 |
| 13 | 31.0 | 4.74E0 |
| 14 | 27.4 | 4.85E0 |
| 15 | 20.7 | 1.11E1 |

TABLE 6
Tablet Compositions, 10 mg

| | Content | Amount per |
|---|---|---|
| Component | (% w/w) | Tablet (mg) |
| Compound 1 SDD (25% | 20.00 | 40.00 |
| drug load) | | |
| Microcrystalline Cellulose | 25.0 | 95.00 |
| (Avicel PH101)* | | |
| Microcrystalline Cellulose | 22.5 | |
| (Avicel PH200)** | | |
| Lactose Monohydrate | 25.00 | 50.00 |
| Croscarmellose Sodium | 4.50 | 9.00 |
| Sodium Stearyl Fumarate | 3.00 | 6.00 |
| Total | 100.0 | 200.0 |

*Avicel PH101 has a particle size of about 50 microns.
**Avicel PH200 has a particle size of about 180 microns.

Tablets having the compositions set forth in Tables 7-10 were prepared by similar methods

TABLE 7
Tablet Composition, 50 mg

| | | % wt Coated | |
|---|---|---|---|
| | Component | Tablet | mg/tablet |
| Intragranular | Compound 1 SDD | 48.54 | 200.0 |
| blend | (25% drug load) | | |
| | Microcrystalline | 22.57 | 93.0 |
| | Cellulose (Avicel PH101) | | |
| | Croscarmellose sodium | 1.46 | 6.0 |
| | Magnesium stearate | 0.24 | 1.0 |
| Extragranular | Microcrystalline | 22.09 | 91.0 |
| blend | Cellulose (Acivel PH102) | | |
| | Croscarmellose sodium | 1.46 | 6.0 |
| | Magnesium stearate | 0.73 | 3.0 |
| | Core Tablet Total | | 400.0 |
| | Opadry Blue | 2.91 | 12.0 |
| | Final Tablet Total | 100.00 | 412.0 |

TABLE 8
Tablet Composition, 50 mg

| | | % wt Core | |
|---|---|---|---|
| | Component | Tablet | mg/tablet |
| Intragranular | Compound 1 SDD | 50.0 | 200.0 |
| blend | (25% drug load) | | |
| | Microcrystalline | 23.4 | 93.6 |
| | Cellulose (Avicel PH101) | | |
| | Croscarmellose sodium | 1.5 | 6.0 |
| | Magnesium stearate | 0.1 | 0.4 |
| Extragranular | Microcrystalline | 22.6 | 90.4 |
| blend | Cellulose (Avicel PH102) | | |
| | Croscarmellose sodium | 1.5 | 6.0 |
| | Magnesium stearate | 0.9 | 3.6 |
| | Core Tablet Total | 100.0 | 400.0 |
| | Opadry Blue | | 12.0 |
| | Final Tablet Total | | 412.0 |

TABLE 9
Tablet Composition, 50 mg

| Component | mg/tablet |
|---|---|
| Compound 1 SDD (25% drug load) | 200.0 |
| Microcrystalline Cellulose | 184.0 |
| Croscarmellose sodium | 12.0 |
| Magnesium stearate | 4.0 |
| Opadry Blue | 12.0 |
| Core Tablet Total (no Opadry Blue) | 400.0 |
| Final Tablet Total (with Opadry Blue) | 412.0 |

TABLE 10
Tablet Composition, 23 mg

| | | % wt Core | |
|---|---|---|---|
| | Component | Tablet | mg/tablet |
| Intragranular | Compound 1 SDD | 20.00 | 92.00 |
| blend | (25% drug load) | | |
| | Microcrystalline | 25.00 | 115.00 |
| | Cellulose (Avicel PH101) | | |
| | Lactose Fast Flo 316 | 25.00 | 115.00 |
| | Croscarmellose sodium | 3.00 | 13.80 |
| | Sodium Stearyl Fumarate | 2.00 | 9.20 |
| Extragranular | Microcrystalline | 22.50 | 103.50 |
| blend | Cellulose (Acivel PH102) | | |
| | Croscarmellose sodium | 1.50 | 6.90 |
| | Sodium Stearyl Fumarate | 1.00 | 4.60 |
| | Core Tablet Total | 100.00 | 460.0 |

### Example 3

### Preparation of Compound 1 SDD Tablet by Direct Compression

Tablets having the composition set forth in Table 11 were prepared. Compound 1 SDD (which can be prepared as described in Example 2, Step 1) and mannitol were each passed through 032R Comil® screen. Separately, microcrystalline cellulose, colloidal silicon dioxide, croscarmellose sodium were each passed through a 30 mesh screen. The ingredients were then combined in a 30 cuft V-shell blender. The mixture was blended for about 12 minutes at 15 rpm. Magnesium stearate was passed through a 40 mesh screen and added to the blended mixture, and the mixture was further blended for about 5 minutes at 15 rpm.

The resulting mixture was then compressed into tablets using a KORSCH® tablet press to yield tablets with each containing 70 mg of Compound 1. The press was equipped with 0.33×0.65 inch modified-oval tooling and paddle feeder. The turret speed was set to 40 rpm and the paddle speed was set to 20 rpm. The target weight and hardness of the tablets is summarized in Table 12.

TABLE 11
Tablet Composition, 70 mg

| | % w/w | % w/w | |
|---|---|---|---|
| | Core | Final | |
| Component | Tablet | Tablet | mg/tablet |
| Compound 1 SDD | 50.0 | 48.544 | 280.00 |
| (25% w/w Compound 1, 75% w/w | | | |
| HPMCAS (see Example 2, Step 1)) | | | |
| Microcrystalline Cellulose | 22.9 | 22.233 | 128.24 |
| Mannitol (spray-dried) | 22.9 | 22.233 | 128.24 |
| Croscarmellose sodium | 3.0 | 2.913 | 16.80 |
| Magnesium stearate | 1.0 | 0.971 | 5.60 |
| Colloidal silicon dioxide | 0.2 | 0.194 | 1.12 |
| Core Tablet Total | 100.0 | 97.088 | 560.00 |
| Opadry II Blue coating | | 2.913 | 16.80 |
| Final Tablet Total | | 100.0 | 576.80 |

*Sum of individual component weight percentages is not precisely 100% w/w due to rounding of individual component weight percentages.

TABLE 12

| | Weight (mg) | Hardness (kP) |
|---|---|---|
| Target Acceptance | 560.0 | 18.0 |
| Limits | 537.6-582.4 | 11.0-26.0 |

After the tablets were made, they were charged into a 48” ACCELA COTA pan coater and coated with Opadry II 85F105201 blue coating to a weight gain of 3% (Range: 2.5-6%). The resulting tablets were analyzed via X-ray powder diffraction to confirm absence of crystalline Compound 1. Tablets were also tested for product identification, product appearance, assay, dissolution, water content, content uniformity, degradation products, disintegration and friabiltiy,

### Example 4

A Study of the Efficacy and Safety of Compound 1 in Subjects with Pain Following Bunionectomy

A randomized, double-blind, placebo-controlled, 5-arm, parallel-design study to evaluate the efficacy and safety of Compound 1 on acute surgical pain is conducted. Bunionectomy is a well-established, multi-dose, surgical, acute pain model. A randomized, double-blind study design was used to avoid observer bias and reduce symptoms or outcomes arising from the subjects' knowledge of treatment. An opioid reference arm assessing a standard-of-care treatment (hydrocodone bitartrate (5 mg)/acetaminophen (325 mg) (HB/APAP)) was included to establish the ability of the study to successfully observe a treatment effect for Compound 1.

### Study Subjects

Male and female patients between the ages of 18 and 75 years (inclusive) with pain that is ≥4 on an 11-point Numeric Pain Rating Scale (NPRS) and is moderate or severe on the Verbal Categorical Rating Scale (VRS) after bunionectomy are included in the study.

### Study Drugs

Investigational Drug: Compound 1. The investigational drug was administered orally in 10 mg tablets. Tablets were prepared according to Example 2, Table 6. The investigational drug was administered every 12 hours (q12h). A low, mid, and high dosing regimen were tested. The first dose in the low dosing regime was 20 mg, and the subsequent doses were 10 mg g12h. The first dose in the mid dosing regime was 60 mg, and the subsequent doses were 30 mg g12h. The first dose in the high dosing regime was 100 mg, and the subsequent doses were 50 mg g12h.

Reference Drug: HB/APAP. The reference drug was administered orally in 5 mg/325 mg capsules, supplied as over-encapsulated 5 mg/325 mg tablets. The reference drug was administered in a dose of 5 mg/325 mg every 6 hours (q6h).

### Study Protocol

The schedule for the study is summarized in Table 13. After a screening period, subjects receive a primary unilateral first metatarsal bunionectomy repair on Day minus one under regional anesthesia (Mayo and popliteal block). A continuous popliteal sciatic block infusion (0.2% ropivacaine) is started after surgery, and remains in place until approximately 3 AM on Day 1. After removal of the popliteal sciatic block, each subject is randomized when the subject requests the first dose of study drug for pain relief and reports pain ≥4 on the NPRS and moderate or severe pain on the VRS. The NPRS and VRS criteria are designed to ensure subjects have sufficient pain to determine if the study drugs are effective. Subjects that do not meet the NPRS and VRS criteria within 9 hours of removal of the popliteal sciatic block are not enrolled in the study.

TABLE 13
Study Schedule

| Study Day | Event |
|---|---|
| Day −28 | Beginning of screening period |
| Day −1 | Bunionectomy |
| Day 1 | Removal of popliteal block |
| (approximately 3 AM) | |
| Day 1 | Randomization |
| Days 1-3 | Administration of study drug |
| Days 12-16 | Safety follow-up phone interview |

264 subjects were randomized approximately 2:2:1:2:2 to five treatment groups: Compound 1 (high dose); Compound 1 (mid dose); Compound 1 (low dose); HB/APAP (opioid reference); or placebo (see Table 14). Randomization was stratified by site and baseline NPRS (<8 versus ≥8). To maintain the blind, all subjects receive the same number of capsules in a double-dummy design.

TABLE 14
Treatment Groups

| | | Number of |
|---|---|---|
| Treatment | Active Dose | Subjects |
| Compound 1 | 100 mg first dose, then 50 mg q12 h | 60 |
| (high dose) | | |
| Compound 1 | 60 mg first dose, then 30 mg q12 h | 62 |
| (mid dose) | | |
| Compound 1 | 20 mg first dose, then 10 mg q12 h | 33 |
| (low dose) | | |
| HB/APAP | 5 mg/325 mg q6 h | 60 |
| Placebo | | 59 |

Compound 1 was administered every 12 hours (q12h). The final dose of Compound 1 is given 36 hours after the first dose. HB/APAP placebo capsules were administered every 6 hours (q6h).

In the Reference Arm, HB/APAP was administered every 6 hours (q6h) at a dose of 5 mg/325 mg. The final dose of HB/APAP was given 42 hours after the first dose. Compound 1 placebo capsules are administered every 12 hours (q12h).

In the Placebo Arm, Compound 1 placebo capsules were administered every 12 hours (q12h), and HB/APAP placebo capsules were administered every 6 hours (q6h).

Subjects had a Safety Follow-up Phone Interview 14 (±2) days after the last study drug dose for the purpose of collecting information on adverse events, medications, and treatments and procedures.

### Efficacy Assessments

11-point (0 to 10) Numeric Pain Rating Scale: NPRS scores are frequently used in bunionectomy studies and are recognized by the FDA as a valid pain intensity measure. On the 11-point NPRS, a score of 0 denotes no pain, and a score of 10 denotes the worst pain intensity imaginable. Subjects reported their pain on the 11-point NPRS immediately before their first dose of the study drug (baseline NPRS score) and at intervals over 48 hour period (i.e. 0.5, 1, 1.5, 2, 3, 4, 5, 6, 8, 12, 16, 20, 24, 28, 32, 36, 40, 44, and 48 hours (±5 minutes)) after the first dose of the study drug. Pain intensity is also recorded on the NPRS immediately before each administration of rescue medication. Pain intensity scores that are collected within 4 hours after a dose of rescue medication are deemed missing, and the missing values are imputed as the intensity recorded immediately before the administration of rescue medication.

SPID-48 is the sum of the pain intensity difference (PID) over the 48 hour time period. A pain intensity score of 0 (no pain) to 10 (worse possible pain) is obtained before starting the study and throughout the 48 time period. The pain score at each assessment time is subtracted from the baseline pain score to provide the total sum score or SPID-48. A higher SPID-48 is better and indicates a reduction in pain intensity compared to the baseline score.

4-point Verbal Categorical Rating Scale: Subjects report their pain on the 4-point VRS (none, mild, moderate, or severe) immediately before their first dose of the study drug (baseline VRS).

### Efficacy Results

SPID48 Scores. The SPID scores for the subjects in the Placebo Arm, Investigational Arm, and Reference Arm appear Table 15 below.

TABLE 15
Bunionectomy Efficacy Results

| | | | | | Hydrocodone |
|---|---|---|---|---|---|
| | | High-dose | Mid-dose | Low-dose | bitartrate/ |
| | | Compound 1 | Compound 1 | Compound 1 | acetaminophen |
| | | (100 mg first | (60 mg first | (20 mg first | reference arm |
| | | dose/50 mg | dose/30 mg | dose/10 mg | (5 mg/325 mg |
| Treatment | Placebo | every 12 hours) | every 12 hours) | every 12 hours) | every six hours) |
| Group: | n = 59 | n = 60 | n = 62 | n = 33 | n = 60 |
| Mean SPID48 | 101.0 | 137.8 | 86.9 | 112.9 | 115.6 |
| MeanSPID48 | N/A | 36.8 | −14.1 | 11.9 | 14.7 |
| difference | | | | | |
| from placebo | | | | | |
| p-value vs. | N/A | p = 0.0251 | p = 0.3859 | p = 0.5379 | p = 0.3706 |
| placebo | | | | | |

The bunionectomy study met its primary endpoint, showing a statistically significant improvement in SPID48, as recorded on a Numeric Pain Rating Scale (NPRS), for those treated with Compound 1 at the high dose compared to placebo. Higher SPID48 values represent greater improvements in pain relief The onset of action was rapid and was sustained through the duration of assessment.

### Safety Results

Safety evaluations include adverse events, clinical laboratory assessments, clinical evaluation of vital signs, electrocardiograms, and physical examinations. No patients discontinued treatment due to AEs and no patients had SAEs. All AEs were mild or moderate. The most common AEs (incidence >10% in either placebo, HB/APAP or Compound 1 high-dose group, respectively) were headache (12%, 7%, 8%) and nausea (9%, 18%, 8%).

Compound 1 was also studied in patients undergoing abdominoplasty and demonstrated statistically significant improvement in SPID48 as well (NCT05034952).

### Example 5

Study of the Efficacy and Safety of Compound 1 in Subjects with Acute Pain after a Bunionectomy

A randomized, double-blind, placebo-controlled, 3-arm, parallel study evaluating the efficacy and safety of Compound 1 in treating acute pain after a bunionectomy was conducted. Bunionectomy is a well-established, multi-dose, surgical, acute pain model. A randomized, double-blind study design was selected to avoid observer bias and reduce symptoms or outcomes arising from the subjects' knowledge of treatment. A parallel design was considered most appropriate given the acute nature of bunionectomy surgery. An opioid reference group assessing a standard-of-care treatment (HB/APAP 5 mg/325 mg q6h) was included to establish the ability of the study, as executed, to successfully observe a treatment effect.

### Study Subjects

Male and female subjects, 18 through 80 years of age (inclusive), with pain that is moderate or severe on the verbal categorical rating scale (VRS) and ≥4 on the numeric pain rating scale (NPRS) after a bunionectomy were included in the study.

### Study Drugs

Investigational Drug: Compound 1. The investigational drug (50 mg tablets) and matching placebo were administered orally. Tablets had the formulation described in Table 8 and were prepared according to Example 2.

Reference Drug: Hydrocodone bitartrate/acetaminophen (HB/APAP). The reference drug (5 mg/325 mg capsules (over-encapsulated tablets)) and matching placebo were administered orally.

### Study Protocol

The schedule for the study is summarized in Table 16. After a screening period, subjects underwent a primary unilateral bunionectomy with distal first metatarsal osteotomy (i.e., Austin procedure) and internal fixation under regional anesthesia (Mayo and popliteal sciatic block) on Day −1. A continuous popliteal sciatic block infusion (0.2% ropivacaine) was started after surgery and remained in place until approximately 3 AM, but no later than 5 AM, on Day 1. After removal of the popliteal sciatic block, a subject was randomized to 1 of 3 treatment groups if: (1) the subject requested the first dose of study drug for pain relief, (2) the subject's pain was moderate or severe on the VRS, and (3) the subject's pain was ≥4 on the NPRS. If a subject did not meet the VRS and NPRS criteria within 9 hours after removal of the popliteal sciatic block, the subject was not eligible for this study.

TABLE 16
Study Schedule

| Study Day | Event |
|---|---|
| Day −28 | Beginning of screening period |
| Day −1 | Bunionectomy |
| Day 1 | Removal of popliteal block |
| (approximately 3 AM) | |
| Day 1 | Randomization |
| Days 1-3 | Administration of study drug, with first |
| | dose administered within 25 minutes after |
| | randomization |
| Days 15-19 | Safety follow-up visit |

Approximately 1000 subjects were randomized 2:2:1 to 3 treatment groups: Compound 1 (100 mg first dose, then 50 mg every 12 hours [q12h]), HB/APAP (5 mg/325 mg every 6 hours [q6h]; opioid reference), or placebo (Table 17). Randomization was stratified by site and baseline NPRS (<8 versus ≥8). To maintain the blind, all subjects received the same number of tablets and capsules q6h in a double-dummy design.

TABLE 17
Treatment Groups

| | | Number of | Number of |
|---|---|---|---|
| | | Subjects | Subjects |
| Treatment | Active Dose | (planned) | (actual) |
| Compound 1 | 100 mg first dose, | 400 | 426 |
| | then 50 mg q12 h | | |
| HB/APAP | 5 mg/325 mg q6 h | 400 | 431 |
| Placebo | | 200 | 216 |

Notes:
To maintain the blind, all subjects received the same number of tablets and capsules in a double-dummy design.
Compound 1 active or Compound 1 placebo tablets were administered to all subjects q12 h as follows: 0 hours (first dose) and at 12, 24, and 36 hours after the first dose of study drug. HB/APAP active or HB/APAP placebo capsules were administered to all subjects q6 h as follows: 0 hours (first dose) and at 6, 12, 18, 24, 30, 36, and 42 hours after the first dose of study drug.

Subjects reported their pain intensity on the NPRS at each scheduled time point through 48 hours after the first dose of study drug. In addition, pain intensity was recorded on the NPRS immediately before each administration of rescue medication.

Ibuprofen (400 mg orally q6h as needed) was permitted as a rescue medication for pain relief upon the subject's request starting any time after the first dose of study drug through 48 hours after the first dose of study drug. Subjects were encouraged to wait 90 minutes after the first dose of study drug to request rescue medication, and subjects generally were not to receive rescue medication unless their NPRS score was ≥4.

### Efficacy and Safety Assessments

Verbal Categorical Rating Scale (VRS). After removal of the popliteal sciatic block, subjects reported their pain on the VRS upon request for the first dose of study drug for pain relief. A 4-point VRS (none, mild, moderate, or severe) was included as part of the pain threshold inclusion criterion to ensure that all subjects had moderate or severe pain at baseline, which ensured subjects had sufficient pain to determine if the study drug is effective.

Numeric Pain Rating Scale (NPRS). After removal of the popliteal sciatic block, subjects reported their pain intensity on the NPRS if pain was rated moderate or severe on the VRS. During the Treatment Period, subjects reported their pain intensity on the NPRS at intervals through 48 hours after the first dose of study drug (i.e., 0.5, 1, 1.5, 2, 3, 4, 5, 6, 8, 12, 16, 20, 24, 28, 32, 36, 40, 44, and 48 hours after the first dose). In addition, pain intensity was recorded on the NPRS immediately before each administration of rescue medication. NPRS is frequently used in bunionectomy studies and is recognized by the FDA as a valid pain intensity measure. Pain rated ≥4 on an 11-point (0 to 10) NPRS at baseline ensures subjects have sufficient pain to determine if the study drug is effective.

Use of Rescue Medications. A record was kept of all rescue medication use. An unscheduled NPRS was completed immediately before each administration of rescue medication. The use of rescue medications is an important covariate in pain studies that can impact the analysis of other efficacy assessments.

Patient Global Assessment (PGA) of Study Drug. The PGA of study drug was completed 48 hours after the first dose of study drug, before the start of any other assessments scheduled at the same time point. The PGA of study drug is a single-item assessment of patient perceptions of pain control with the study drug and is evaluated on a 4-point Likert scale (poor, fair, good, or excellent). The PGA of study drug provides subjects the ability to state their perceptions of the treatment that may not be captured by measuring pain intensity alone.

Safety. Safety evaluations included adverse events, clinical laboratory assessments, clinical evaluation of vital signs, standard 12-lead electrocardiograms, and physical examinations (PEs).

### Study Endpoints

Study endpoints included:
Time-weighted sum of the pain intensity difference (SPID) as recorded on the numeric pain rating scale (NPRS) from 0 to 48 hours (SPID48) compared to placebo;
SPID48 compared to hydrocodone bitartrate/acetaminophen (HB/APAP);
Time to >2-point reduction in NPRS from baseline compared to placebo;
Time to >1-point reduction in NPRS from baseline compared to placebo;
Proportion of subjects reporting good or excellent on the Patient Global Assessment (PGA) at 48 hours compared to placebo;
Incidence of vomiting or nausea compared to HB/APAP;
Time-weighted SPID as recorded on the NPRS from 0 to 24 hours (SPID24) compared to placebo;
Time to first use of rescue medication compared to placebo;
Proportion of subjects using rescue medication from 0 to 48 hours compared to placebo;
Total rescue medication usage from 0 to 48 hours compared to placebo;
Safety and tolerability based on the incidence and type of adverse events (AEs), changes from baseline in clinically significant laboratory test results, vital signs, and ECGs;
Time-weighted SPID as recorded on the NPRS from 0 to 36 hours (SPID36) compared to placebo;
Time-weighted SPID as recorded on the NPRS from 0 to 12 hours (SPID12) compared to placebo;
Proportion of subjects with ≥30% reduction in NPRS at 48 hours compared to placebo;
Proportion of subjects with ≥50% reduction in NPRS at 48 hours compared to placebo;
Proportion of subjects with ≥70% reduction in NPRS at 48 hours compared to placebo;
Proportion of subjects using rescue medication from 0 to 24 hours compared to placebo;
Time to first use of rescue medication in the first 12 hours compared to placebo
Proportion of subjects using rescue medication from 0 to 12 hours compared to placebo; and
Proportion of subjects using rescue medication from 24 to 48 hours compared to placebo.

### Efficacy Results

SPID48 Scores. The time-weighted sum of the pain intensity difference (SPID) as recorded on the numeric pain rating scale (NPRS) from 0 to 48 hours (SPID48) for Compound 1 versus placebo and for Compound 1 versus HB/APAP is reported in Tables 18 and 19, respectively.

As shown in Table 18, Compound 1 showed a statistically significant and clinically meaningful greater reduction in pain compared to placebo.

TABLE 18
SPID48 - Compound 1 versus Placebo

| | Placebo | Compound 1 |
|---|---|---|
| | n = 216 | n = 426 |
| LS mean (SE) | 70.6 (6.3) | 99.9 (4.5) |
| LS mean difference from placebo | — | 29.3 |
| 95% CI | — | (14.0, 44.6) |
| P value vs. placebo | — | 0.0002 |

As shown in Table 19, Compound 1 did not show a statistically significant greater reduction in pain compared to HB/APAP.

TABLE 19
SPID48 - Compound 1 versus HB/APAP

| | HB/APAP | Compound 1 |
|---|---|---|
| | n = 431 | n = 426 |
| LS mean (SE) | 120.1 (4.5) | 99.9 (4.5) |
| LS mean difference from HB/APAP | — | −20.2 |
| 95% CI | — | (−32.7, −7.7) |
| P value vs. HB/APAP | — | 0.0016 |

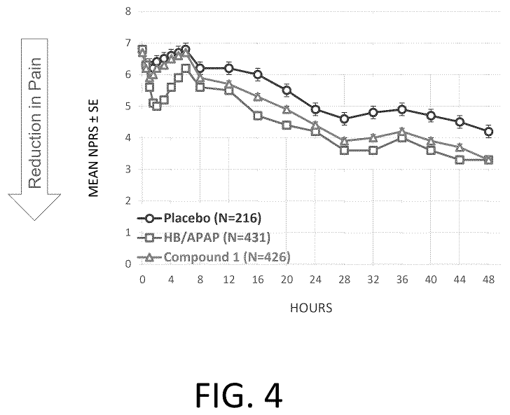

NPRS Scores. The mean NPRS scores over time in each treatment group are shown in FIG. 4. NPRS scores at baseline (mean) and changes from baseline in NPRS scores at 48 hours (mean) for each treatment group are reported in Table 20. As shown in Table 20, a clinically meaningful reduction in pain (a ≥2-point reduction in NPRS) from baseline was observed in the Compound 1 treatment group.

TABLE 20
NPRS Scores (Baseline and 48 Hours)

| | Placebo | HB/APAP | Compound 1 |
|---|---|---|---|
| | n = 216 | n = 431 | n = 426 |
| Baseline NPRS, mean | 6.8 | 6.8 | 6.7 |
| Change from baseline in | −2.6 | −3.6 | −3.4 |
| NPRS at 48 hours, mean | | | |
| % reduction from baseline | 38% | 53% | 51% |
| in mean NPRS at 48 hours | | | |

Time to ≥2-Point Reduction in NPRS. The proportion of subjects achieving meaningful pain relief (a ≥2-point reduction in NPRS) and the median time to 2-point reduction in NPRS from baseline for each treatment group are reported in Table 21. As shown in Table 21, Compound 1 showed a faster median time to 2-point reduction in NPRS from baseline (4 hours), compared to placebo (8 hours).

TABLE 21
Time to ≥2-Point Reduction in NPRS

| | Placebo | HB/APAP | Compound 1 |
|---|---|---|---|
| | N = 216 | N = 431 | N = 426 |
| Proportion of subjects | 82.4% | 92.3% | 90.1% |
| achieving ≥2-point reduction | | | |
| in NPRS from baseline | | | |
| Time to ≥2-point reduction | | | |
| in NPRS from baseline (min) | | | |
| Median, 95% CI | 480 | 90 | 240 |
| | (476, 716) | (85, 110) | (117, 477) |
| P value vs. | — | — | 0.0016 |
| placebo (Log-rank) | | | |

### Safety Results

Compound 1 was generally safe and well tolerated in subjects with bunionectomy, and the overall safety profile was similar to placebo. The majority of adverse events were mild or moderate in severity. There were no serious adverse events and no adverse events leading to treatment discontinuation in any treatment group. No clinically relevant changes in clinical laboratory measurements, vital signs, or ECG parameters were observed in any treatment group.

The majority of common adverse events had a lower incidence in the Compound 1 group than the HB/APAP group. For example, the incidence of nausea/vomiting was lower in the Compound 1 group (9.2%) than in the HB/APAP group (16.5%). Adverse events with an incidence ≥5% in any treatment group (Compound 1, placebo, HB/APAP, respectively) were nausea (8.2%, 10.6%, 14.4%), headache (4.9%, 9.3%, 10.4%), constipation (3.5%, 4.2%, 5.1%), and dizziness (3.5%, 5.1%, 5.3%).

### Example 6

Study of the Efficacy and Safety of Compound 1 in Subjects with Acute Pain after an Abdominoplasty

A randomized, double-blind, placebo-controlled, 3-arm, parallel study design evaluating the efficacy and safety of Compound 1 in treating acute pain after an abdominoplasty was conducted. Abdominoplasty is a well-established, multi-dose, surgical, acute pain model. A randomized, double-blind study design was selected to avoid observer bias and reduce symptoms or outcomes arising from the subjects' knowledge of treatment. A parallel design was considered most appropriate given the acute nature of abdominoplasty surgery. An opioid reference group assessing a standard-of-care treatment (HB/APAP 5 mg/325 mg q6h) was included to establish the ability of the study, as executed, to successfully observe a treatment effect.

### Study Subjects

Male and female subjects, 18 through 80 years of age (inclusive), with pain that is moderate or severe on the verbal categorical rating scale (VRS) and ≥4 on the NPRS after an abdominoplasty were included in the study.

### Study Drugs

Investigational Drug: Compound 1. The investigational drug (50 mg tablets) and matching placebo were administered orally. Tablets had the formulation described in Table 8 and were prepared according to Example 2.

Reference Drug: Hydrocodone bitartrate/acetaminophen (HB/APAP). The reference drug (5 mg/325 mg capsules (over-encapsulated tablets)) and matching placebo were administered orally.

### Study Protocol

The schedule for the study is summarized in Table 22. After a screening period, subjects underwent a standard (“full”) abdominoplasty procedure on Day 1. After surgery completion, a subject was randomized to 1 of 3 treatment groups if: (1) the subject requested the first dose of study drug for pain relief, (2) the subject's pain was moderate or severe on the VRS, and (3) the subject's pain was ≥4 on the NPRS. If a subject did not meet the VRS and NPRS criteria within 4 hours after surgery completion, the subject was not eligible for this study.

TABLE 22
Study Schedule

| Study Day | Event |
|---|---|
| Day −28 | Beginning of screening period |
| Day 1 | Abdominoplasty |
| Day 1 | Randomization |
| Days 1-3 | Administration of study drug, with first dose |
| | administered within 25 minutes after randomization |
| Days 15-19 | Safety follow-up visit |

Approximately 1000 subjects were randomized 2:2:1 to 3 treatment groups: Compound 1 (100 mg first dose, then 50 mg every 12 hours [q12h]), HB/APAP (5 mg/325 mg every 6 hours [q6h]; opioid reference), or placebo (Table 23). Randomization was stratified by site and baseline NPRS (<8 versus ≥8). To maintain the blind, all subjects received the same number of tablets and capsules q6h in a double-dummy design.

TABLE 23
Treatment Groups

| | | Number of | Number of |
|---|---|---|---|
| | | Subjects | Subjects |
| Treatment | Active Dose | (planned) | (actual) |
| Compound 1 | 100 mg first dose, | 400 | 447 |
| | then 50 mg q12 h | | |
| HB/APAP | 5 mg/325 mg q6 h | 400 | 448 |
| Placebo | | 200 | 223 |

Notes:
To maintain the blind, all subjects received the same number of tablets and capsules in a double-dummy design.
Compound 1 active or Compound 1 placebo tablets were administered to all subjects q12 h as follows: 0 hours (first dose) and at 12, 24, and 36 hours after the first dose of study drug. HB/APAP active or HB/APAP placebo capsules were administered to all subjects q6 h as follows: 0 hours (first dose) and at 6, 12, 18, 24, 30, 36, and 42 hours after the first dose of study drug.

Subjects reported their pain intensity on the NPRS at each scheduled time point through 48 hours after the first dose of study drug. In addition, pain intensity was recorded on the NPRS immediately before each administration of rescue medication.

Ibuprofen (400 mg orally q6h, as needed) was permitted as a rescue medication for pain relief upon the subject's request starting any time after the first dose of study drug through 48 hours after the first dose of study drug. Subjects were encouraged to wait 90 minutes after the first dose of study drug to request rescue medication, and subjects generally were not to receive rescue medication unless their NPRS score was ≥4.

### Efficacy and Safety Assessments

Verbal Categorical Rating Scale (VRS). Subjects reported their pain on the VRS upon request for the first dose of study drug for pain relief. A 4-point VRS (none, mild, moderate, or severe) was included as part of the pain threshold inclusion criterion to ensure that all subjects had moderate or severe pain at baseline, which ensured subjects had sufficient pain to determine if the study drug is effective.

Numeric Pain Rating Scale (NPRS). Subjects reported their pain intensity on the NPRS upon request for the first dose of study drug for pain relief if pain was rated moderate or severe on the VRS. During the Treatment Period, subjects reported their pain intensity on the NPRS at intervals through 48 hours after the first dose of study drug (i.e., 0.5, 1, 1.5, 2, 3, 4, 5, 6, 8, 12, 16, 20, 24, 28, 32, 36, 40, 44, and 48 hours after the first dose). In addition, pain intensity was recorded on the NPRS immediately before each administration of rescue medication. NPRS is frequently used in abdominoplasty studies and is recognized by the FDA as a valid pain intensity measure. Pain rated ≥4 on an 11-point (0 to 10) NPRS at baseline ensures subjects have sufficient pain to determine if the study drug is effective.

Use of Rescue Medications. A record was kept of all rescue medication use. An unscheduled NPRS was completed immediately before each administration of rescue medication. The use of rescue medications is an important covariate in pain studies that can impact the analysis of other efficacy assessments.

Patient Global Assessment (PGA) of Study Drug. The PGA of study drug was completed 48 hours after the first dose of study drug, before the start of any other assessments scheduled at the same time point. The PGA of study drug is a single-item assessment of patient perceptions of pain control with the study drug and is evaluated on a 4-point Likert scale (poor, fair, good, or excellent). The PGA of study drug provides subjects the ability to state their perceptions of the treatment that may not be captured by measuring pain intensity alone.

Safety. Safety evaluations included adverse events, clinical laboratory assessments, clinical evaluation of vital signs, standard 12-lead electrocardiograms, and physical examinations (PEs).

### Study Endpoints

Study endpoints included:
Time-weighted sum of the pain intensity difference (SPID) as recorded on the numeric pain rating scale (NPRS) from 0 to 48 hours (SPID48) compared to placebo;
SPID48 compared to hydrocodone bitartrate/acetaminophen (HB/APAP);
Time to 2-point reduction in NPRS from baseline compared to placebo;
Time to 1-point reduction in NPRS from baseline compared to placebo;
Proportion of subjects reporting good or excellent on the Patient Global Assessment (PGA) at 48 hours compared to placebo;
Incidence of vomiting or nausea compared to HB/APAP;
Time-weighted SPID as recorded on the NPRS from 0 to 24 hours (SPID24) compared to placebo;
Time to first use of rescue medication compared to placebo;
Proportion of subjects using rescue medication from 0 to 48 hours compared to placebo;
Total rescue medication usage from 0 to 48 hours compared to placebo;
Safety and tolerability based on the incidence and type of adverse events (AEs), changes from baseline in clinically significant laboratory test results, vital signs, and ECGs;
Time-weighted SPID as recorded on the NPRS from 0 to 36 hours (SPID36) compared to placebo;
Time-weighted SPID as recorded on the NPRS from 0 to 12 hours (SPID12) compared to placebo;
Proportion of subjects with ≥30% reduction in NPRS at 48 hours compared to placebo;
Proportion of subjects with ≥50% reduction in NPRS at 48 hours compared to placebo;
Proportion of subjects with ≥70% reduction in NPRS at 48 hours compared to placebo;
Proportion of subjects using rescue medication from 0 to 24 hours compared to placebo;
Time to first use of rescue medication in the first 12 hours compared to placebo;
Proportion of subjects using rescue medication from 0 to 12 hours compared to placebo; and
Proportion of subjects using rescue medication from 24 to 48 hours compared to placebo.

### Efficacy Results

SPID48 Scores. The time-weighted sum of the pain intensity difference (SPID) as recorded on the numeric pain rating scale (NPRS) from 0 to 48 hours (SPID48) for Compound 1 versus placebo and for Compound 1 versus HB/APAP is reported in Tables 24 and 25, respectively.

As shown in Table 24, Compound 1 showed a statistically significant and clinically meaningful greater reduction in pain compared to placebo.

TABLE 24
SPID48 - Compound 1 versus Placebo

| | Placebo | Compound 1 |
|---|---|---|
| | n = 223 | n = 447 |
| LS mean (SE) | 70.1 (6.1) | 118.4 (4.3) |
| LS mean difference from placebo | — | 48.4 |
| 95% CI | — | (33.6, 63.1) |
| P value vs. placebo | — | <0.0001 |

As shown in Table 25, Compound 1 did not show a statistically significant greater reduction in pain compared to HB/APAP.

TABLE 25
SPID48 - Compound 1 versus HB/APAP

| | HB/APAP | Compound 1 |
|---|---|---|
| | n = 448 | n = 447 |
| LS mean (SE) | 111.8 (4.3) | 118.4 (4.3) |
| LS mean difference from | — | 6.6 |
| HB/APAP | | |
| 95% CI | — | (−5.4, 18.7) |
| P value vs. HB/APAP | — | 0.2781 |

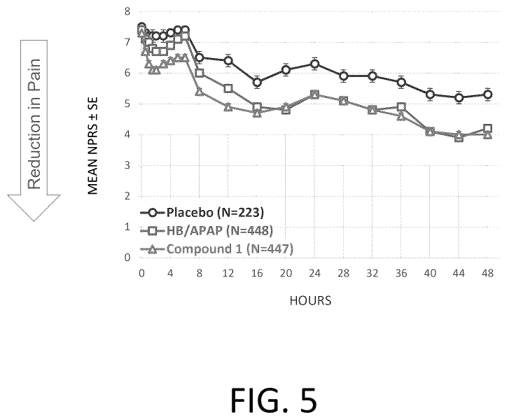

NPRS Scores. The mean NPRS scores over time in each treatment group are shown in FIG. 5. NPRS scores at baseline (mean) and changes from baseline in NPRS scores at 48 hours (mean) for each treatment group are reported in Table 26. As shown in Table 26, a clinically meaningful reduction in pain (a ≥2-point reduction in NPRS) from baseline was observed in the Compound 1 treatment group.

TABLE 26
NPRS Scores (Baseline and 48 Hours)

| | Placebo | HB/APAP | Compound 1 |
|---|---|---|---|
| | n = 223 | n = 448 | n = 447 |
| Baseline NPRS, mean | 7.5 | 7.4 | 7.3 |
| Change from | −2.3 | −3.2 | −3.4 |
| baseline in NPRS at | | | |
| 48 hours, mean | | | |
| % reduction from | 31% | 43% | 47% |
| baseline in mean | | | |
| NPRS at 48 hours | | | |

Time to ≥2-Point Reduction in NPRS. The proportion of subjects achieving meaningful pain relief (a ≥2-point reduction in NPRS) and the median time to ≥2-point reduction in NPRS from baseline for each treatment group are reported in Table 27. As shown in Table 27, Compound 1 showed a faster median time to ≥2-point reduction in NPRS from baseline (2 hours), compared to placebo (8 hours).

TABLE 27
Time to ≥2-Point Reduction in NPRS

| | Placebo | HB/APAP | Compound 1 |
|---|---|---|---|
| | N = 223 | N = 448 | N = 447 |
| Proportion of | 76.2% | 86.6% | 90.6% |
| subjects achieving ≥2- | | | |
| point reduction in | | | |
| NPRS from baseline | | | |
| Time to ≥2-point | | | |
| reduction in NPRS | | | |
| from baseline (min) | | | |
| Median, 95% CI | 408 (477, 705) | 475 (236, 480) | 119 (90, 180) |
| P value vs. | — | — | <0.0001 |
| placebo (Log-rank) | | | |

### Safety Results

Compound 1 was generally safe and well tolerated in subjects with abdominoplasty, and the overall safety profile was similar to placebo. The majority of adverse events were mild or moderate in severity, and there were no serious adverse events related to Compound 1. The incidence of adverse events leading to treatment discontinuation was low and balanced among treatment groups (0.5%, 1.1%, 1.1% in the placebo, HB/APAP, and Compound 1 groups, respectively). No clinically relevant changes in clinical laboratory measurements, vital signs, or ECG parameters were observed in any treatment group.

The majority of common adverse events had a lower incidence in the Compound 1 group than the HB/APAP group. For example, the incidence of nausea/vomiting was lower in the Compound 1 group (20.3%) than in the HB/APAP group (33.5%). Adverse events with an incidence ≥5% in any treatment group (Compound 1, placebo, HB/APAP, respectively) were nausea (19.0%, 25.2%, 32.8%) constipation (10.5%, 10.8%, 8.7%), headache (4.2%, 5.0%, 7.1%), dizziness (4.0%, 7.7%, 5.4%) and hypotension (2.5%, 6.8%, 3.6%).

### Example 7

### Single-Arm Study to Evaluate Safety and Effectiveness of Compound 1 for Acute Pain

A single-arm study evaluating the safety and effectiveness of Compound 1 in treating acute pain was conducted.

### Study Subjects

Male and female subjects between the ages of 18 and 80 years, inclusive, with pain that was moderate or severe on a verbal categorical rating scale (VRS) and ≥4 on a numeric pain rating scale (NPRS) resulting from an ambulatory surgical procedure (e.g., inguinal hernia repair, hemorrhoidectomy, or shoulder arthroscopy) or non-surgical conditions (e.g., traumatic acute musculoskeletal pain), with pain that was expected to last for at least 72 hours and that was expected to require no more than a short-term (i.e., <24-hour) admission, if any, were included in the study.

### Study Drug

Investigational Drug: Compound 1. The investigational drug (50 mg tablets) was administered orally. Tablets had the formulation described in Table 8 and were prepared according to Example 2.

### Study Protocol

This was a single-arm study evaluating the safety and effectiveness of Compound 1 in treating acute pain in surgical and non-surgical subjects. The schedule for the study is summarized in Table 28 (surgical subjects) and Table 29 (non-surgical subjects).

Surgical Subjects. On Day 1, subjects underwent a scheduled ambulatory surgical procedure with pain that was expected to last for at least 72 hours and that was expected to require no more than a short-term (i.e., <24-hour) admission. After surgery completion, a subject was assigned to treatment if (1) the subject requested the first dose of study drug for pain relief, (2) the subject's pain was moderate or severe on the VRS, and (3) the subject's pain was ≥4 on the NPRS. If a subject did not meet the VRS and NPRS criteria 12 hours (procedures performed with regional anesthesia) or 4 hours (all other procedures) after surgery completion, the subject was not eligible for this study.

Non-Surgical Subjects. On Day 1, after completion of screening assessments and confirmation of eligibility, a subject began treatment if (1) the subject's pain was moderate or severe on the VRS, and (2) the subject's pain was ≥4 on the NPRS. For a subject to be eligible, their pain must have been ongoing for ≤48 hours at presentation.

All Subjects. Approximately 250 subjects received Compound 1 (100 mg first dose, then 50 mg every 12 hours [q12h]). Subjects continued to receive Compound 1 for 14 days or until their pain resolved, whichever occurred first. An acetaminophen/ibuprofen combination was permitted to be used as a rescue medication for pain relief as needed (prn), starting any time after the first dose of study drug through Day 14, completion of study drug treatment due to pain resolution, or study drug discontinuation, whichever occurred first. Subjects were permitted to take acetaminophen 650 mg/ibuprofen 400 mg every 6 hours (q6h) prn, up to a maximum of 2600 mg/1600 mg in any 24-hour period.

TABLE 28
Study Schedule (Surgical Subjects)

| Study Day | Event |
|---|---|
| Day −28 | Beginning of screening period |
| Day 1 | Surgical procedure |
| Day 1 | Request for study drug |
| Days 1-14 (or until pain resolves, | Administration of study drug |
| whichever occurs first) | |
| Days 26-30 | Safety follow-up visit |

TABLE 29
Study Schedule (Non-Surgical Subjects)

| Study Day | Event |
|---|---|
| Day 1 | Treatment assignment |
| Days 1-14 (or until pain resolves, | Administration of study drug |
| whichever occurs first) | |
| Days 26-30 | Safety follow-up visit |

The following approach was used to define pain resolution and completion of study drug treatment:
1. Pain was considered to be resolved and the study drug treatment was considered completed if
   a. a subject took no study drug during a 48-hour period (i.e., skipped at least 4 consecutive doses due to pain resolution); and
   b. a subject took no more than 1 dose of acetaminophen/ibuprofen during each 24-hour period within the 48-hour period (i.e., no more than 2 doses in 48 hours).
2. If a subject stopped taking study drug per Criterion 1a but did not meet Criterion 1b, they were instructed to restart study drug (and prn rescue medication).
3. If a subject stopped and restarted study drug (Criterion 2 above) and subsequently believed their pain was resolving and stopped study drug a second time, they were instructed to not restart study drug, regardless of whether the above criteria were met.
4. If a subject stopped study drug a second time after the restart of dosing (Criterion 3 above) and then fulfilled both Criterion 1a and 1b, the pain was considered resolved and study drug treatment was considered completed. If the subject fulfilled only Criterion 1a but not Criterion 1b after the restart of dosing, the pain was considered not resolved and study drug was considered discontinued.

Note: If subject stopped study drug due to pain resolution less than 48 hours before the last scheduled dose of study drug (i.e., stopped after the second dose on Day 12) and therefore could not meet Criterion 1a by Day 14, the subject was considered to have completed study drug dosing as long as they also met Criterion 1b.

### Efficacy and Safety Assessments

Verbal Categorical Rating Scale (VRS). Subjects reported their pain on the VRS upon request for the first dose of study drug for pain relief after surgery completion (surgical subjects) or before treatment assignment (non-surgical subjects).

Numeric Pain Rating Scale (NPRS). Subjects reported their pain intensity on the NPRS upon request for the first dose of study drug for pain relief after surgery completion if pain was rated moderate or severe on the VRS (surgical subjects) or immediately after the VRS and before treatment assignment if the subject's pain was rated moderate or severe on the VRS (non-surgical subjects).

Use of Rescue Medications. A record was kept of all rescue medication use. The use of rescue medications is an important covariate in pain studies that can impact the analysis of other efficacy assessments.

Patient Global Assessment (PGA) of Study Drug. The PGA of study drug was completed on day 2 (after the third study drug dose, approximately 24 hours after the first dose), on day 7 (after the first study drug dose on study day 7), and on day 14 (after final study drug dose for subjects still taking study drug on day 14; within approximately 24 hours after the last or presumed last dose of study drug for subjects who have discontinued or completed study drug treatment). The PGA captures subjects' perceptions of the study drug's effectiveness in treating pain using a validated, single-item questionnaire. This study used a 5-point Likert scale (poor, fair, good, very good, or excellent) for the PGA evaluation.

Safety. Safety evaluations included adverse events, clinical laboratory assessments, clinical evaluation of vital signs, standard 12-lead electrocardiograms, and physical examinations (PEs).

### Study Endpoints

Study endpoints included:
Safety and tolerability based on treatment-emergent adverse events (TEAEs), laboratory test results, vital signs, and electrocardiograms; and
Subject perception of Compound 1 effectiveness in treating pain at the end of treatment as measured by the proportion of subjects reporting good, very good, or excellent on a patient global assessment (PGA).

### Safety and Efficacy Results

A total of 256 subjects, including 222 surgical subjects and 34 non-surgical subjects, participated in the study.

Compound 1 was generally safe and well tolerated for up to 14 days in subjects with surgical and non-surgical pain. The majority of adverse events were mild or moderate in severity. The incidence of serious adverse events (0.8%) and treatment discontinuations due to adverse events (2.0%) were low, and no serious adverse events were related to Compound 1. The safety profile was consistent with that of the studies described in Examples 4 and 5.

Patient perception of Compound 1 effectiveness in treating pain, as measured by the Patient Global Assessment (PGA) at the end of treatment, showed that 83.2% of patients (including 82.0% of surgical patients and 91.2% of non-surgical patients) reported Compound 1 as good, very good, or excellent.

### Example 8

Study of the Efficacy and Safety of Compound 1 in subjects with Painful Diabetic Peripheral Neuropathy

A randomized, double-blind, active-controlled, dose-ranging, 4-arm, parallel-design study to evaluate the safety and efficacy of Compound 1 in treating subjects with painful diabetic peripheral neuropathy (DPN) was conducted.

### Study Subjects

The study population included male and female subjects 18 through 80 years of age, inclusive, with presence of bilateral pain in lower extremities due to DPN for at least 1 year, and weekly average Numeric Pain Rating Scale (NPRS) pain score of ≥4.

### Study Design

Subjects who met eligibility criteria entered a 7-day Run-in Period to establish their baseline Numeric Pain Rating Scale (NPRS) pain score and Daily Sleep Interference Scale (DSIS) score. Subjects with a baseline average NPRS score of ≥4 points with limited variation (SD<25% of mean) were eligible for the 12-week Treatment Period followed by a Safety Follow-up Visit.

A total of approximately 175 subjects were randomized 2:2:1:2 to 4 treatment arms: Compound 1 (high, mid, or low dose) or pregabalin (reference arm) (Table 30). Randomization was stratified by sex (female and male) and body mass index (≥30 and <30 kg/m²). To maintain the blind, all subjects received the same number of tablets once daily (qd) in the morning and the same number of capsules 3 times per day (tid) in a double-dummy design. After the Treatment Period, subjects tapered off capsule (pregabalin reference or matched placebo) study drug for 7 days (4 days of dosing every 12 hours, then 3 days of dosing qd), and the safety follow up visit occurred an additional 7 (±2) days later.

TABLE 30
Treatment Arms

| | | Number of | Number of |
|---|---|---|---|
| | | Subjects | Subjects |
| Treatment | Active Dose | (Planned) | (Actual) |
| Compound 1 (high dose) | 69 mg qd | 50 | 50 |
| Compound 1 (mid dose) | 46 mg qd | 50 | 50 |
| Compound 1 (low dose) | 23 mg qd | 25 | 24 |
| Pregabalin | 100 mg tid | 50 | 49 |

qd: once daily;
tid: 3 times per day
Note:
To maintain the blind, all subjects received the same number of tablets and the same number of capsules at the same respective frequency (i.e., qd for tablets and tid for capsules during the Treatment Period) in a double-dummy design.

Subjects stopped taking pain medications (including pregabalin, if applicable), except acetaminophen (500 mg), for at least 14 days before the first dose of study drug.

Acetaminophen was be permitted as a pain rescue medication as needed (prn) throughout the study. Subjects were permitted to take 500 mg of acetaminophen every 4 to 6 hours as needed, up to a maximum of 2500 mg in any 24-hour period. Subjects recorded rescue medication use, and their current pain score on the NPRS immediately before each administration of rescue medication.

### Study Drugs

Investigational Drug: Compound 1. The investigational drug was administered orally in a tablet containing 23 mg of active ingredient. Tablets were prepared according to Example 2, Table 10. The investigational drug was administered once daily. A low, mid, and high dosing regime were tested, as described in Table 30.

Reference Drug: Pregabalin. Pregabalin is an anticonvulsant approved for the treatment of painful diabetic neuropathy; it is considered first line treatment in most international clinical guidelines and forms a key part of management of neuropathic pain. The reference drug was administered orally in a 100 mg capsule tid. The doses and dose frequency are summarized in Table 31 below.

TABLE 31
Study Drug

| | Dosing Form/ | | |
|---|---|---|---|
| Drug Name | Route | Dosage | How Supplied |
| Compound 1 | Tablet/oral | 23, 46, or 69 | Supplied as 23-mg tablets |
| | | mg qd | |
| Placebo | Tablet/oral | 0 mg qd | Supplied as tablets |
| Pregabalin | Capsule/oral | 100 mg tid | Supplied as 100-mg |
| | | | capsules |
| Pregabalin | Capsule/oral | 0 mg tid | Supplied as capsules |
| placebo | | | |

### Efficacy Assessments

11-point (0 to 10) Numeric Pain Rating Scale (NPRS): NPRS scores are recognized by the FDA as a valid pain intensity measure and are used as a standard pain assessment scale in many pain registration studies. On the 11-point NPRS, a score of 0 denotes no pain, and a score of 10 denotes the worst pain intensity imaginable.

From Day −7 through Week 12, subjects reported their average daily pain during the last 24 hours on the NPRS via e-diary; during the treatment period, the NPRS was completed in the morning before the first daily dose. Subjects also reported their current pain score before each administration of rescue medication. The NPRS scores from the daily e-diary, especially the change from baseline in the weekly average at Week 12, were used in the primary endpoint analysis, and the proportion of subjects with ≥30%, ≥50%, and ≥70% reduction in weekly average scores, especially at Week 12, as reported in the daily e-diary were used in the secondary endpoint analyses. For the primary endpoint, daily NPRS scores were averaged over a weekly period to reduce the impact of individual high or low pain scores.

Daily Sleep Interference Scale (DSIS): Pain frequently interferes with sleep, and sleep is important to quality of life. The FDA recommends evaluating the effect of analgesics on sleep. DSIS is commonly used in neuropathic pain studies and is assessed on an 11-point scale. The 11 point scale ranges from 0 (none) to 10 (severe). The DSIS was completed each morning before the first daily dose in an e-diary to describe how pain interfered with the subject's sleep. The change from baseline in the weekly average of DSIS, especially at Week 12, was used in the secondary endpoint analysis.

Patient Global Impression of Change (PGIC): The PGIC is commonly used in neuropathic pain studies, and the Initiative on Methods, Measurement, and Pain Assessment in Clinical Trials (IMMPACT) group recommends it as a core outcome measure for chronic pain studies and is thus evaluated in this study. There is some evidence that PGIC may be more sensitive in neuropathic pain studies than pain intensity assessments because it may assess additional quality of life measures. The assessment consists of a single item on a 7-point scale from 1 (very much improved) to 7 (very much worse). The PGIC was completed at selected study visits to quantify the change in subjects' overall status. The proportion of subjects categorized as improved on the PGIC assessment, especially at Week 12, was used in the secondary endpoint analysis.

Proportion of subjects using Rescue Medication and Total Medication Usage: Acetaminophen was permitted as a pain rescue medication as needed (prn) throughout the study. In an e-diary, subjects recorded rescue medication use and their current pain score on the NPRS immediately before each administration of rescue medication. Data on the use of rescue medications was collected for descriptive analysis across the 4 treatment groups, and the proportion of subjects using the rescue medication and the total medication usage was used as an endpoint.

### Efficacy Results

Baseline Scores. The baseline NPRS and DSIS scores for the subjects in the treatment arms were reported. Baseline values for the NPRS daily pain intensity scores and DSIS were defined as the average score from Day −7 to Day −1. For all other variables, baseline values were defined as the most recent non-missing measurement collected before the first dose of study drug. Change (absolute change) from baseline was calculated as [post-baseline value]−[baseline value].

NPRS Scores. A statistically significant and clinically meaningful reduction in pain from baseline was observed at week 12 within all three Compound 1 dose groups. The mean changes from baseline in weekly average of daily pain on the NPRS at week 12 are reported in Table 32. All Compound 1 dose groups had significant mean reduction in pain from baseline that started from week 1. The pain continued to decrease until week 5, and then sustained throughout the rest of the treatment period.

TABLE 32
NPRS Scores

| | Compound 1 | Compound 1 | Compound 1 | |
|---|---|---|---|---|
| | 23 mg qd | 46 mg qd | 69 mg qd | Pregabalin |
| | (low dose) | (mid dose) | (high dose) | 100 mg tid |
| Baseline NPRS | 5.70 | 5.88 | 5.79 | 5.98 |
| (Mean (SD)) | (1.32) | (0.97) | (1.22) | (1.28) |
| Change from | −2.18 | −2.11 | −2.26 | −2.09 |
| baseline at week 12 | (0.39) | (0.28) | (0.28) | (0.29) |
| (LS Mean (SE)) | | | | |
| 95% CI | (−2.94, −1.41) | (−2.67, −1.55) | (−2.82, −1.70) | (−2.65, −1.52) |
| P value | <0.0001 | <0.0001 | <0.0001 | <0.0001 |

DSIS Scores. An improvement in sleep was observed at week 12 within all three Compound 1 dose groups.

Statistical Analysis. The primary efficacy endpoint was the change from baseline in the weekly average of the daily pain intensity on an NPRS at Week 12. The primary analysis was a within-group comparison in any Compound 1 dose group.

The primary efficacy analysis was based on a mixed-effects model for repeated measures (MMRM), with change from baseline in weekly average of daily pain intensity score as the dependent variable; and fixed effects of treatment group, time (categorical), treatment group-by-time interaction, baseline weekly average of daily pain intensity, and baseline weekly average of daily pain intensity-by-time interaction. The least squares (LS) mean change from baseline at Week 12 for each group was presented with the corresponding SE, and the corresponding 95% confidence interval and P value.

The proportion of subjects with ≥30% reduction in the weekly average of daily pain intensity on the NPRS at Week 12 was summarized descriptively by treatment group and analyzed using the Cochran-Mantel-Hanzel test (stratified by sex and BMI). The proportion of subjects with ≥50% and ≥70% reduction was analyzed similarly. The proportion of subjects categorized as improved at Week 12 on the PGIC assessment was summarized descriptively by treatment group and analyzed using the Cochran-Mantel-Hanzel test (stratified by sex and BMI). The change from baseline in the weekly average of the DSIS at Week 12 was analyzed similarly to the primary endpoint. The changes from baseline in NPSI and SF-MPQ-2 at each time point were analyzed using an MMRM. The changes from baseline in SF-36 and BDI-2 at 12 weeks were analyzed by an analysis of covariance (ANOVA) model. The use of rescue medication was summarized descriptively with the percentage of subjects using rescue medication and the total medication usage. No statistical testing was conducted on the safety endpoints.

### Safety Assessments and Results

Safety evaluations included adverse events, clinical laboratory assessments, clinical evaluation of vital signs, electrocardiograms, physical examinations, and Columbia Suicide Severity Rating Scale (C-SSRS).

Compound 1 was found to be safe and well tolerated at all studied doses for up to 12 weeks of treatment. The majority of adverse events were mild or moderate in severity. Incidence of severe adverse events was low and balanced between subjects treated with Compound 1 and subjects treated with pregabalin. None of the severe adverse events were considered to be related to study drug.

The most common adverse events (incidence >5% in either Compound 1 combined or pregabalin group, respectively) were creatinine clearance decrease (5.1%, 1.9%), dizziness (0.7%, 9.3%), peripheral edema (0.7%, 5.6%) and weight increased (0%, 7.4%). Related adverse events were 14.5% in patients treated with Compound 1 and 27.8% in patients treated with pregabalin.

### Example 9

Study of the Efficacy and Safety of Compound 1 in subjects with Painful Diabetic Peripheral Neuropathy

A randomized, double-blind, active-controlled, parallel-design study to evaluate the safety and efficacy of Compound 1 in treating subjects with painful diabetic peripheral neuropathy (DPN) is conducted.

### Study Subjects

The study population includes male and female subjects 18 through 80 years of age, inclusive, with presence of bilateral pain in lower extremities due to DPN for at least 1 year, and weekly average Numeric Pain Rating Scale (NPRS) pain score of ≥4.

### Study Design

Subjects who meet eligibility criteria enter a 7-day Run-in Period to establish their baseline Numeric Pain Rating Scale (NPRS) pain score. Subjects with a baseline average NPRS score of ≥4 points with limited variation (SD<25% of mean) are eligible for the 12-week Treatment Period followed by a Safety Follow-up Visit.

A total of approximately 608 subjects are randomized 1:1 to 2 treatment arms: Compound 1 (investigational arm) or pregabalin (reference arm) (Table 33). Randomization is stratified by sex (female and male) and body mass index (≥30 and <30 kg/m²). To maintain the blind, all subjects receive the same number of tablets once daily (qd) in the morning and the same number of capsules 3 times per day (tid) in a double-dummy design. After the Treatment Period, subjects taper off capsule (pregabalin reference or matched placebo) study drug for 7 days (4 days of dosing every 12 hours, then 3 days of dosing qd), and the safety follow up visit occurs an additional 7 (±2) days later.

TABLE 33
Treatment Arms

| | | Number of Subjects |
|---|---|---|
| Treatment | Active Dose | (Planned) |
| Compound 1 | 70 mg qd | 304 |
| Pregabalin | 100 mg tid | 304 |

qd: once daily;
tid: 3 times per day
Note:
To maintain the blind, all subjects receive the same number of tablets and the same number of capsules at the same respective frequency (i.e., qd for tablets and tid for capsules during the Treatment Period) in a double-dummy design.

Subjects stop taking pain medications (including pregabalin, if applicable), except acetaminophen (500 mg every 4 to 6 hours, as needed), for at least 14 days before the first dose of study drug.

Acetaminophen is be permitted as a pain rescue medication prn throughout the study. Subjects are permitted to take 500 mg every 4 to 6 hours, as needed, up to a maximum of 2500 mg in any 24-hour period. Subjects record rescue medication use.

### Study Drugs

Investigational Drug: Compound 1. The investigational drug is administered orally in a tablet having a formulation described in Example 2 or a similar formulation. The investigational drug is administered once daily. The dose and dose frequency are summarized in Table 34 below.

Reference Drug: Pregabalin. Pregabalin is an anticonvulsant approved for the treatment of painful diabetic neuropathy; it is considered first line treatment in most international clinical guidelines and forms a key part of management of neuropathic pain. The reference drug is administered orally in a 100 mg capsule tid. The dose and dose frequency are summarized in Table 34 below.

TABLE 34
Study Drug

| | Dosing Form/ | | |
|---|---|---|---|
| Drug Name | Route | Dosage | How Supplied |
| Compound 1 | Tablet/oral | 70 mg qd | Supplied as tablets |
| Placebo | Tablet/oral | 0 mg qd | Supplied as tablets |
| Pregabalin | Capsule/oral | 100 mg tid | Supplied as 100-mg |
| | | | capsules |
| Pregabalin | Capsule/oral | 0 mg tid | Supplied as capsules |
| placebo | | | |

### Efficacy Assessments

11-point (0 to 10) Numeric Pain Rating Scale (NPRS): From Day −7 through Week 12, subjects report their average daily pain during the last 24 hours on the NPRS via e-diary; during the treatment period, the NPRS is completed in the morning before the first daily dose. Subjects also report their current pain score before each administration of rescue medication. The NPRS scores from the daily e-diary, especially the change from baseline in the weekly average at Week 12, are used in the primary endpoint analysis, and the proportion of subjects with ≥30%, ≥50%, and ≥70% reduction in weekly average scores, especially at Week 12, as reported in the daily e-diary are used in the secondary endpoint analyses.

Daily Sleep Interference Scale (DSIS): The DSIS is completed each morning before the first daily dose in an e-diary to describe how pain interfered with the subject's sleep. The change from baseline in the weekly average of DSIS, especially at Week 12, is used in the secondary endpoint analysis.

Patient Global Impression of Change (PGIC): The PGIC is completed at selected study visits to quantify the change in subjects' overall status. The proportion of subjects categorized as improved on the PGIC assessment, especially at Week 12, is used in the secondary endpoint analysis.

Neuropathic Pain Symptom Inventory (NPSI): The NPSI is completed at selected study visits to quantify the change in subjects' distinct pain symptoms. The change from baseline in the total intensity score on the NPSI, especially at Week 12, is used as an endpoint.

Short-form McGill Pain Questionnaire-2 (SF-MPQ-2): The SF-MPQ-2 is completed at selected site visits to quantify the change in subjects' overall and specific pain symptoms. The change from baseline in pain characteristics and intensity using the SF-MPQ-2, especially at Week 2, is used as an endpoint.

SF-36: The SF-36 is completed to quantify the impact on subjects' overall health status. The change from baseline in the SF-36, especially at Week 12, is used as an endpoint.

BDJ: The BDJ-2 is completed to quantify the change in subjects' emotional functioning. The change from baseline in the BDI, especially at Week 12, is used as an endpoint.

Bedside Sensory Testing Kit (BSTK): The BSTK is used for the purpose of standardized assessment and phenotyping of subjects' pain, using the usual components of a neurosensory examination and is thus evaluated in this study.

Proportion of subjects using Rescue Medication and Total Medication Usage: Acetaminophen is permitted as a pain rescue medication as needed (prn) throughout the study. In an e-diary, subjects record rescue medication use and their current pain score on the NPRS immediately before each administration of rescue medication. Data on the use of rescue medications is collected for descriptive analysis across the 2 treatment groups, and the proportion of subjects using the rescue medication and the total medication usage is used as an endpoint.

### Efficacy Results

Baseline Scores. The baseline NPRS and DSIS scores for the subjects in the treatment arms are reported. Baseline values for the NPRS daily pain intensity scores and DSIS are defined as the average score from Day −7 to Day −1. For all other variables, baseline values are defined as the most recent non-missing measurement collected before the first dose of study drug. Change (absolute change) from baseline is calculated as [post-baseline value]−[baseline value].

Statistical Analysis. The primary efficacy analysis is based on a mixed effects model for repeated measures (MMRM), with change from baseline in weekly average of daily pain intensity score as the dependent variable, and fixed effects of treatment group, week (categorical), treatment group-by-week interaction, baseline weekly average of daily pain intensity, and baseline weekly average of daily pain intensity-by-week interaction. The least squares (LS) mean difference from pregabalin in the primary endpoint is presented with the corresponding 95% CI and P value.

### Safety Assessments and Results

Safety evaluations include adverse events, clinical laboratory assessments, clinical evaluation of vital signs, electrocardiograms, physical examinations, and Columbia Suicide Severity Rating Scale (C-SSRS).

### Example 10

Evaluation of Efficacy and Safety of Compound 1 for Pain Associated with Diabetic Peripheral Neuropathy

A randomized, double-blind, placebo- and active-controlled, parallel-design study to evaluate the efficacy and safety of Compound 1 in treating subjects with pain associated with diabetic peripheral neuropathy (DPN) is conducted.

### Study Subjects

The study population includes male and female subjects 18 through 80 years of age, inclusive, with presence of bilateral pain in lower extremities due to DPN for at least 1 year, and weekly average Numeric Pain Rating Scale (NPRS) pain score of ≥4 and ≤9 with limited variation in the Baseline Period. Subjects have a diagnosis of diabetes mellitus type 1 or type 2 and (a) have glycosylated hemoglobin Alc (HbAlc)≤9% and (b) have optimized glycemic control and are stable on anti-diabetic medicine/drugs or dietary treatment for ≥3 months before Screening Visits.

### Study Design

Excluding the Screening Period (which occurs within 35 days before administration of study drug and includes a 7-day Baseline Period), each subject participates in the study for approximately 14 weeks: a 12-week Treatment Period and a Safety Follow-up Visit 14 (±2) days after the last dose of tablet study drug.

Subjects who meet eligibility criteria during Screening Visits enter a 7-day Baseline Period. During the Baseline Period, subjects report their average daily pain on the Numeric Pain Rating Scale (NPRS) to establish their baseline NPRS pain score. Subjects with a baseline weekly average NPRS score of ≥4 and ≤9 points with limited variation (SD<25% of mean) are eligible for the 12-week Treatment Period followed by a Safety Follow-up Visit.

A total of approximately 1100 subjects are randomized 1:1:1 to 3 treatment arms: Compound 1 (investigational arm), pregabalin (reference arm), or placebo (placebo arm) (Table 35). Randomization is stratified by study site and screening creatinine clearance level (<80 or ≥80 mL/min). To maintain the blind, all subjects receive the same number of tablets once daily (qd; 1 tablet) and the same number of capsules 3 times per day (tid; 1 capsule) in a double-dummy design for the 12-week Treatment Period. After the 12-week Treatment Period, subjects enter a 7-day period to allow for capsule (pregabalin or matched placebo) study drug taper.

TABLE 35
Treatment Arms

| | | Number of Subjects |
|---|---|---|
| Treatment | Active Dose | (Planned) |
| Compound 1 | 70 mg qd | 367 |
| Pregabalin | 100 mg tid* | 367 |
| Placebo | | 367 |

qd: once daily;
tid: 3 times per day
*Note:
Subjects randomized to active pregabalin will receive 50 mg tid for the first week of dosing, then 100 mg tid until beginning the taper regimen.

A Safety Follow-up Visit occurs 14 (±2) days after the last dose of tablet study drug. The Safety Follow-up Visit is not required for subjects who enroll in (i.e., sign the informed consent form) a separate Compound 1 open-label extension study by or on the day of the Week 12 Visit. To be eligible for this open-label study, subjects must complete study drug treatment during the Treatment Period and complete the Week 12 Visit and subsequent 7-day period (to allow for capsule study drug taper) and should continue to take blinded tablet (Compound 1 or matched placebo) study drug during the 7-day period. A subject continuing to take any dose of capsule study drug after the end of the 7-day period is not eligible to enter the open-label study.

Subjects stop taking pain medications (including pregabalin, if applicable), except acetaminophen (500 mg every 4 to 6 hours, as needed), for at least 14 days before the first dose of study drug.

Acetaminophen is be permitted as a pain rescue medication prn throughout the study. Subjects are permitted to take 500 mg every 4 to 6 hours, as needed, up to a maximum of 2500 mg on any given calendar day. Subjects record NPRS pain score immediately before each administration of rescue medication and record date, time, and dose (in mg) of administration.

### Study Drugs

Investigational Drug: Compound 1. The investigational drug is administered orally in 70 mg tablets having a formulation described in Example 3 (Table 11). Matching placebo tablets are also administered orally. The dosage forms and routes of administration are summarized in Table 36 below.

Reference Drug: Pregabalin. Pregabalin (an anticonvulsant) is a first-line, oral, FDA-approved therapy for DPN. The reference drug is administered orally in 50- and 100-mg capsules. Matching placebo tablets are also administered orally. The dosage forms and routes of administration are summarized in Table 36 below.

TABLE 36
Study Drugs

| | Dosing Form/ | | |
|---|---|---|---|
| Drug Name | Route | Dosage | How Supplied |
| Compound 1 | Tablet/oral | 70 mg qd | Supplied as tablets |
| Placebo | Tablet/oral | 0 mg qd | Supplied as tablets |
| Pregabalin | Capsule/oral | 100 mg tid | Supplied as 50- and |
| | | | 100-mg capsules |
| Pregabalin | Capsule/oral | 0 mg tid | Supplied as capsules |
| placebo | | | |

### Efficacy, Safety, and Pharmacokinetic Assessments

11-point (0 to 10) Numeric Pain Rating Scale (NPRS): This evaluation is a standard pain assessment scale used in many pain registration studies. The 11-point scale ranges from 0 (no pain) to 10 (worst imaginable pain). From Day −7 (the beginning of the 7-day Baseline Period) through Week 12, subjects report their average daily pain during the last 24 hours on the NPRS via e-diary; during the treatment period, the NPRS is completed in the morning before the first daily dose. Subjects also report their current pain score before each administration of rescue medication.

Medical Outcomes Study 36-item Short-form Health Status (SF-36v2)—Physical Component Summary (PCS) and Mental Component Summary (MCS): The SF-36v2 is a 36-item scale, which measures 8 domains of general health status: physical functioning, role limitations due to physical problems, social functioning, bodily pain, mental health, role limitations due to emotional problems, vitality, and general health perception. Physical and mental component summary scores (PCS and MCS, respectively) are calculated based on weighting the scores of the 8 domains.

Safety: Safety evaluations include adverse events, clinical laboratory assessments (including serum chemistry, hematology, coagulation, and urinalysis), clinical evaluation of vital signs, electrocardiograms (ECGs), physical examinations, and Columbia Suicide Severity Rating Scale (C-SSRS).

Pharmacokinetics: Pharmacokinetic evaluations include determination of pharmacokinetic parameters of Compound 1 and its metabolite.

Patient Global Assessment (PGA): The PGA captures subjects' perceptions of the study drug using a single-item questionnaire measured using a validated Likert scale. PGAs are generic measures that have precedence in clinical studies for chronic pain conditions.

Profile of Mood States (POMS-2-SF): The POMS-2-SF was developed to measure the effects of mood on various therapeutic approaches. The domains measured are tension-anxiety, depression, anger-hostility, vigor-activity, fatigue, and confusion. Total mood disturbance is a function of these six domain scores and reported as a total score. The short form has been validated for patients under stress and pain.

Work Productivity and Activity Impairment—Neuropathic Pain (WPAI-NP): The WPAI-NP is a validated and broadly used 6-item instrument to measure the impact of an individual's health status on their work and daily activities. It measures the impact of neuropathic pain on absenteeism, presenteeism and overall work impairment as a function of these two domains. The WPAI-NP also measures impairments in daily activities other than work at a job and reports this as a separate domain.

Brief Pain Inventory—Short-form (BPI-SF): The BPI-SF is widely used in clinical and research settings with patients who have chronic pain. The BPI-SF questionnaire assesses both pain severity (pain severity index) and the impact of pain on daily life, such as its interference with walking and sleep, (pain interference index) in two separate index scores.

Short-form McGill Pain Questionnaire-2 (SF-MPQ-2): The SF-MPQ-2 is designed to measure the multidimensional qualities of pain as well as total pain by assessing the major symptoms of both neuropathic and non-neuropathic pain. It assesses 15 specific sensory and affective pain descriptors and provides a total score and sensory and affective subscale scores.

Neuropathic Pain Symptom Inventory (NPSI): The NPSI is a well-characterized method for quantifying distinct symptoms experienced by a diversity of neuropathic pain patients as well as treatment effects. The instrument characterizes neuropathic pain symptom severity and is comprised of 5 subscales: superficial spontaneous pain, deep (or pressing) spontaneous pain, paroxysmal pain, evoked pain, paresthesia/dysesthesia. A total severity score and scores for each subscale may be evaluated.

### Study Endpoints

Study endpoints include:
Change from baseline in the weekly average of daily pain intensity on the numeric pain rating scale (NPRS) at Week 12 compared to placebo;
Change from baseline in the weekly average of daily pain intensity on the NPRS at Week 12 compared to pregabalin;
Change from baseline in the Medical Outcomes Study 36-item Short-form Health Status (SF-36v2) Physical Component Summary (PCS) score at Week 12 compared to placebo;
Change from baseline in the weekly average of daily pain intensity on the NPRS at Week 12 compared to pregabalin;
Change from baseline in the SF-36v2 PCS score at Week 12 compared to placebo;
Proportion of subjects with ≥30% reduction from baseline in the weekly average of daily pain intensity on the NPRS at Week 12 compared to placebo;
Proportion of subjects with ≥50% reduction from baseline in the weekly average of daily pain intensity on the NPRS at Week 12 compared to placebo;
Proportion of subjects with ≥70% reduction from baseline in the weekly average of daily pain intensity on the NPRS at Week 12 compared to placebo;
Safety and tolerability based on treatment emergent adverse events (TEAEs), laboratory test results, vital signs, and ECGs;
Proportion of subjects with 30% reduction from baseline in the weekly average of daily pain intensity on the NPRS at Week 12 compared to pregabalin;
Proportion of subjects with ≥50% reduction from baseline in the weekly average of daily pain intensity on the NPRS at Week 12 compared to pregabalin;
Proportion of subjects with ≥70% reduction from baseline in the weekly average of daily pain intensity on the NPRS at Week 12 compared to pregabalin;
Change from baseline in the SF-36v2 PCS score at Week 12 compared to pregabalin;
Change from baseline in the SF-36v2 Mental Component Summary (MCS) score at Week 12 compared to pregabalin and placebo;
Change from baseline in pain severity and pain interference indices on the Brief Pain Inventory—Short-form (BPI-SF) at Week 12 compared to pregabalin and placebo;
Change from baseline in the Short-form McGill Pain Questionnaire-2 (SF-MPQ-2) total score at Week 12 compared to pregabalin and placebo;
Change from baseline in the Neuropathic Pain Symptom Inventory (NPSJ) total score at Week 12 compared to pregabalin and placebo;
Proportion of subjects reporting positive responses to a patient global assessment (PGA) at Week 12 compared to pregabalin and placebo;
Change from baseline in total mood disturbance on Profile of Mood States (POMS-2-SF) at Week 12 compared to pregabalin and placebo;
Change from baseline in overall work impairment on Work Productivity and Activity Impairment—Neuropathic Pain (WPAI-NP) at Week 12 compared to pregabalin and placebo; and
PK parameter estimates of Compound 1 and its metabolite.

### Efficacy and Statistical Analyses

Baseline Scores. The baseline NPRSscores for the subjects in the treatment arms are reported. Baseline values for the NPRS daily pain intensity scores are defined as the average score from Day −7 to Day −1. For all other variables, baseline values are defined as the most recent non-missing measurement collected before the first dose of study drug. Change (absolute change) from baseline is calculated as [post-baseline value]−[baseline value].

Statistical Analysis. The primary efficacy analysis is based on a mixed effects model for repeated measures (MMRM), with change from baseline in weekly average of daily pain intensity score as the dependent variable, and fixed effects of treatment group, week (categorical), treatment group-by-week interaction, baseline weekly average of daily pain intensity, and baseline weekly average of daily pain intensity-by-week interaction. The least squares (LS) mean difference from pregabalin in the primary endpoint is presented with the corresponding 95% CI and P value.

### Example 11

### Evaluation of Efficacy and Safety of Compound 1 for Painful Lumbosacral Radiculopathy

A randomized, double-blind, placebo-controlled, parallel-design study of the efficacy and safety of Compound 1 in subjects with painful lumbosacral radiculopathy (PLSR) was conducted.

### Study Subjects

The study population included male and female subjects 18 through 70 years of age, inclusive, with pain in 1 lower extremity due to PLSR for at least 3 months, and weekly average NPRS pain score ≥4 and <10 with limited variation during the Run-in Period.

Diagnosis of PLSR was defined by: (a) Stability in intensity and symptomatology for ≥2 weeks before Screening Visits; (b) No changes to prescribed medicines or treatments for PLSR pain for >4 weeks prior to Screening Visits (with the exception of NSAIDs); (c) Sensory disturbances in a single dermatome only as measured by sensory examination at Screening Visits; and (d) Leg pain worse than back pain in a single dermatome in a single lower limb.

### Study Design

Excluding the Screening Period (which included a 7-day Run-in Period), each subject participated in the study for approximately 14 weeks: a 12-week Treatment Period and a Safety Follow-up Visit 14 (±2) days after the last dose of study drug.

Subjects who met eligibility criteria during Screening Visits (which occurred within 35 days before administration of study drug) entered a 7-day Run-in Period to establish their baseline NPRS leg pain score. Subjects with a baseline average NPRS score of ≥4 and <10 points with limited variation (SD<25% of mean) were eligible for the 12-week Treatment Period followed by a Safety Follow-up Visit.

A total of approximately 200 subjects were randomized 1:1 to Compound 1 or placebo (Table 37). Randomization was stratified by NSAID use at baseline (yes or no).

TABLE 37
Treatment Arms

| | | Number of Subjects |
|---|---|---|
| Treatment | Active Dose | (Planned) |
| Compound 1 | 69 mg qd | 100 |
| Placebo | | 100 |

qd: once daily

Subjects stopped taking pain medications, except acetaminophen (up to 500 mg) ibuprofen, or naproxen for at least 14 days before the first dose of study drug.

Subjects were permitted to take acetaminophen (up to 500 mg) every 4 to 6 hours as needed (prn), up to a maximum of 2500 mg in any 24 hour period as rescue medication. Subjects were asked to record their NPRS pain score immediately before each administration and to record the dose administered (in mg) and the date and time of each use of rescue medication in the e-diary. Subjects using stable doses of over-the-counter ibuprofen or naproxen at the time of the Screening Visits were permitted to continue to take up to the stable daily dose (defined as the same dose taken daily for at least 14 days before Screening Visits) to a maximum of either ibuprofen 400 mg every 6 hours (q6h; max 1600 mg over 24 hours) or naproxen 220 mg every 12 hours (q12h; max 440 mg over 24 hours). Subjects whose use exceeded either 1600 mg per day of ibuprofen or 440 mg per day of naproxen at the first Screening Visit were required to reduce their usage to allowed doses for at least 14 days prior to a second Screening Visit to be eligible for enrollment. Subjects were asked to record total daily NSAID use (number of tablets or capsules used) in the e-diary.

### Study Drug

Compound 1. The investigational drug was administered orally in a tablet containing 23 mg of active ingredient. Tablets were prepared according to Example 2, Table 10. The investigational drug was administered once daily. Matching placebo tablets are also administered orally.

### Efficacy, Safety, and Pharmacokinetic Assessments

Study assessments included some or all of the following:

11-point (0 to 10) Numeric Pain Rating Scale (NPRS): This evaluation is a standard pain assessment scale used in many pain registration studies. The 11-point scale ranges from 0 (no pain) to 10 (worst imaginable pain). Each morning during the 7-day Run-in Period through Week 12 of the Treatment Period, subjects recorded their average daily NPRS score from the previous 24 hours. During the Treatment Period, the NPRS was recorded before dosing.

Daily Sleep Interference Scale (DSIS): DSIS is commonly used in neuropathic pain studies and is assessed on an 11-point numeric rating scale. The 11-point scale ranges from 0 (did not interfere with sleep) to 10 (completely interfered with sleep; unable to sleep due to pain). Each morning during the 7-day Run-in Period and through Week 12 of the Treatment Period, subjects reported on the previous night's sleep using the DSIS. During the Treatment Period, the DSIS was recorded before dosing.

Patient Global Impression of Change assessment (PGIC): The PGIC is commonly used in neuropathic pain studies to assess additional impact on quality of life. The assessment consists of a single item on a 7-point scale from 1 (very much improved) to 7 (very much worse).

Neuropathic Pain Symptom Inventory (NPSI): The NPSI is a well-characterized method for quantifying distinct symptoms experienced by a diversity of neuropathic pain patients as well as treatment effects.

Use of rescue medication (acetaminophen).

Brief Pain Inventory (BPI): The BPI is widely used in clinical and research settings with patients who have chronic pain. The BPI questionnaire assesses both pain severity (pain index) and its functional impact (interference index) in 2 separate sections.

Safety: Safety evaluations included adverse events, clinical laboratory assessments (including serum chemistry, hematology, coagulation, and urinalysis), clinical evaluation of vital signs, electrocardiograms (ECGs), physical examinations, and Columbia Suicide Severity Rating Scale (C-SSRS).

Pharmacokinetics: Pharmacokinetic evaluations included determination of pharmacokinetic parameters of Compound 1 and its metabolite.

Hospital Anxiety and Depression Scale (HADS): The HADS questionnaire is used to assess depression and anxiety in patients with physical health problems as opposed to psychiatric problems. It avoids aspects of depression and anxiety that are also common somatic symptoms of physical disease (i.e., fatigue and sleep). It is comprised of subscales for anxiety and depression, each with 7 questions, which are scored and evaluated independently. It is sensitive to changes resulting from analgesic treatment.

Oswestry Disability Index (ODI): The ODI is used extensively to assess function in activities of daily living, and treatment outcomes, for patients with acute or chronic back pain and spinal disorders.

EuroQoL Questionnaire—5 Dimension—5 Level (EQ-5D-5L): Describes overall health through 5 domains (5D), each with 5 ordered response levels (5L): mobility, self-care, usual activities, pain and discomfort, and anxiety and depression. A unique health state is defined by combining 1 level from each domain, and can show changes in health over time. A second section uses a visual analogue scale to assess overall health.

Sensory examination: PLSR is a clinical diagnosis and includes motor and/or sensory changes. The sensory examination uses instruments to assess different types of nerve fibers involved in sensation.

DNA blood sample (optional): A single blood sample was collected for potential exploratory evaluation of associations between DNA markers with other endpoints, PK, PD, and AEs for subjects who chose to participate in this assessment.

### Study Endpoints

Study endpoints included:
Change from baseline in the weekly average of daily leg pain intensity on a numeric pain rating scale (NPRS) at Week 12;
Change from baseline in the weekly average of the Daily Sleep Interference Scale (DSIS) at Week 12;
Safety and tolerability based on the incidence and type of adverse events (AEs) and changes from baseline in clinically significant laboratory test results, vital signs, and ECGs;
PK parameter estimates of Compound 1 and its metabolite;
Proportion of subjects categorized as much improved or very much improved at Week 12 on the Patient Global Impression of Change assessment (PGIC);
Proportion of subjects with ≥30% reduction from baseline in the weekly average of daily leg pain intensity on the NPRS at Week 12;
Proportion of subjects with ≥50% reduction from baseline in the weekly average of daily leg pain intensity on the NPRS at Week 12;
Proportion of subjects with ≥70% reduction from baseline in the weekly average of daily leg pain intensity on the NPRS at Week 12;
Change from baseline in the Brief Pain Inventory (BPI)—Interference index at Week 12;
Change from baseline in the BPI—Pain index at Week 12;
Change from baseline on the EuroQoL Questionnaire—5 Dimension—5 Level health index (EQ-5D-5L) at Week 12;
Change from baseline score on the Neuropathic Pain Symptom Inventory (NPSI) at Week 12;
Change from baseline in the Hospital Anxiety and Depression Scale (HADS) at Week 12; and
Change from baseline in the Oswestry Disability Index (ODI) at Week 12.

### Efficacy and Statistical Analyses

Baseline Scores. Baseline values, unless specified otherwise, were defined as the average score from Day −7 to Day −1 for NPRS daily leg pain intensity and DSIS. For ECGs, the baseline value was defined as the average of the non-missing pretreatment measurements (triplicate) on Day 1.

For all other variables, baseline values were defined as the most recent non-missing measurement collected before the first dose of study drug. Change (absolute change) from baseline is calculated as [post-baseline value]−[baseline value].

Statistical Analysis. The primary efficacy analysis is intended to be based on a mixed effects model for repeated measures (MMRM), with change from baseline in weekly average of daily leg pain intensity score as the dependent variable, and fixed effects of treatment group, week (categorical), treatment group-by-week interaction, baseline weekly average of daily leg pain intensity, and baseline weekly average of daily leg pain intensity-by-week interaction.

### Example 12

### Evaluation of Efficacy and Safety of Compound 1 for Painful Lumbosacral Radiculopathy

A randomized, double-blind, placebo-controlled, parallel-design study of the efficacy and safety of Compound 1 in subjects with painful lumbosacral radiculopathy (PLSR) is conducted.

### Study Subjects

The study population includes male and female subjects 18 through 70 years of age, inclusive, with pain in 1 lower extremity due to PLSR for >3 months, and weekly average NPRS pain score ≥4 and ≤9 with limited variation during the Baseline Period.

Diagnosis of PLSR is defined by: (a) Stability in intensity and symptomatology for ≥2 weeks before Screening Visits; (b) No changes to prescribed medicines or treatments for PLSR pain for >4 weeks prior to Screening Visits (with the exception of NSAIDs); and (c) Leg pain worse than back pain in a single dermatome in a single lower limb. Subjects have sensory disturbances in a single dermatome only as measured by sensory examination at Screening Visit.

### Study Design

Excluding the Screening Period (which included a 7-day Baseline Period), each subject participates in the study for approximately 14 weeks: a 12-week Treatment Period and a Safety Follow-up Visit 14 (±2) days after the last dose of study drug.

Subjects who meet eligibility criteria during Screening Visits (which occur within 35 days before administration of study drug) enter a 7-day Baseline Period to establish their baseline NPRS leg pain score. Subjects with a baseline average NPRS score of ≥4 and ≤9 points with limited variation (SD<25% of mean) are eligible for the 12-week Treatment Period followed by a Safety Follow-up Visit.

A total of approximately 670 subjects are randomized 1:1 to Compound 1 or placebo (Table 38). Randomization is stratified by nonsteroidal anti-inflammatory drug (NSAID) use (yes or no) and site.

TABLE 38
Treatment Arms

| | | Number of Subjects |
|---|---|---|
| Treatment | Active Dose | (Planned) |
| Compound 1 | 70 mg qd | 335 |
| Placebo | | 335 |

qd: once daily

Subjects stop taking pain medications, except acetaminophen (up to 500 mg every 4 to 6 hours as needed [prn]) ibuprofen, or naproxen for at least 14 days before the first dose of study drug.

Acetaminophen prn is permitted as a pain rescue medication throughout the study. Subjects are permitted to take up to 500 mg every 4 to 6 hours prn, up to a maximum of 2500 mg over 1 calendar day (i.e., between 12:00 AM midnight and the following midnight). Subjects are asked to record their NPRS pain score immediately before each administration and to record the date, time, and dose (in mg) of each administration of rescue medication in the e-diary.

Subjects using stable doses (defined as the same dose taken daily for at least 14 days before Screening Visits) of over-the-counter NSAIDs (ibuprofen or naproxen) at the time of the Screening Visits are permitted to (a) completely stop taking NSAIDs (at least 14 days before the first dose of study drug), or (b) continue to take up to the stable daily dose of the same NSAID to a maximum of either ibuprofen 400 mg every 6 hours (q6h; max 1600 mg over 1 calendar day) or naproxen 220 mg every 12 hours (q12h; max 440 mg over 1 calendar day). Subjects whose use exceeds either 1600 mg per day of ibuprofen or 440 mg per day of naproxen at the first Screening Visit must reduce their usage to allowed doses for at least 14 days prior to a second Screening Visit (and throughout the study), or completely stop NSAID use at least 14 days before the first dose of study drug to be eligible for enrollment. Subjects are asked to record their NSAID use in the e-diary.

### Study Drug

Compound 1. The investigational drug is administered orally in 70 mg tablets having a formulation described in Example 3 (Table 11). The investigational drug is administered once daily. Matching placebo tablets are also administered orally.

### Efficacy, Safety, and Pharmacokinetic Assessments

Study assessments include some or all of the following:

11-point (0 to 10) Numeric Pain Rating Scale (NPRS): This evaluation is a standard pain assessment scale used in many pain registration studies. The 11-point scale ranges from 0 (no pain) to 10 (worst imaginable pain). Each morning during the 7-day Baseline Period through Week 12 of the Treatment Period, subjects record their average daily NPRS score from the previous 24 hours. During the Treatment Period, the NPRS is recorded before dosing.

Daily Sleep Interference Scale (DSIS): DSIS is commonly used in neuropathic pain studies and is assessed on an 11-point numeric rating scale. The 11-point scale ranges from 0 (did not interfere with sleep) to 10 (completely interfered with sleep; unable to sleep due to pain). Each morning during the 7-day Baseline Period and through Week 12 of the Treatment Period, subjects report on the previous night's sleep using the DSIS. During the Treatment Period, the DSIS is recorded before dosing.

Safety: Safety evaluations include adverse events, clinical laboratory assessments (including serum chemistry, hematology, coagulation, and urinalysis), clinical evaluation of vital signs, electrocardiograms (ECGs), physical examinations, and Columbia Suicide Severity Rating Scale (C-SSRS).

Pharmacokinetics: Pharmacokinetic evaluations include determination of pharmacokinetic parameters of Compound 1 and its metabolite.

Patient Global Impression of Change assessment (PGIC): The PGIC is commonly used in neuropathic pain studies to assess the patient perception of the overall benefit of treatment over the course of the study. The assessment consists of a single item on a 7-point Likert scale from 1 (very much improved) to 7 (very much worse).

Medical Outcomes Study 36-item Short-form Health Status (SF-36v2)—Physical Component Summary (PCS) and Mental Component Summary (MCS): The SF-36v2 is a 36-item scale, which measures 8 domains of general health status: physical functioning, role limitations due to physical problems, social functioning, bodily pain, mental health, role limitations due to emotional problems, vitality, and general health perception. Physical and mental component summary scores (PCS and MCS, respectively) are calculated based on weighting the scores of the 8 domains.

Brief Pain Inventory—Short-form (BPI-SF): The BPI-SF is widely used in clinical and research settings with patients who have chronic pain. The BPI-SF questionnaire assesses both pain severity (pain severity index) and the impact of pain on daily life, such as its interference with walking and sleep, (pain interference index) in two separate index scores.

EuroQoL Questionnaire—5 Dimension—5 Level (EQ-5D-5L): Describes overall health through 5 domains (5D), each with 5 ordered response levels (5L): mobility, self-care, usual activities, pain and discomfort, and anxiety and depression. A unique health state is defined by combining 1 level from each domain, and can show changes in health over time. A second section uses a visual analogue scale to assess overall health.

Neuropathic Pain Symptom Inventory (NPSI): The NPSI is a well-characterized method for quantifying distinct symptoms experienced by a diversity of neuropathic pain patients as well as treatment effects. The instrument characterizes neuropathic pain symptom severity and is comprised of 5 subscales: superficial spontaneous pain, deep (or pressing) spontaneous pain, paroxysmal pain, evoked pain, paresthesia/dysesthesia. A total severity score and scores for each subscale may be evaluated.

Hospital Anxiety and Depression Scale (HADS): The HADS questionnaire is used to assess depression and anxiety in patients with physical health problems as opposed to psychiatric problems. It avoids aspects of depression and anxiety that are also common somatic symptoms of physical disease (i.e., fatigue and sleep). It is comprised of subscales for anxiety and depression, each with 7 questions, which are scored and evaluated independently. It is sensitive to changes resulting from analgesic treatment.

Oswestry Disability Index (ODI): The ODI is used extensively to assess function in activities of daily living, and treatment outcomes, for patients with acute or chronic back pain and spinal disorders. Lower scores indicate lesser degree of disability impacting the patient's quality of life.

Work Productivity and Activity Impairment—Neuropathic Pain (WPAI-NP): The WPAI-NP is a validated and broadly used 6-item instrument to measure the impact of an individual's health status on their work and daily activities. It measures the impact of neuropathic pain on absenteeism, presenteeism and overall work impairment as a function of these two domains. The WPAI-NP also measures impairments in daily activities other than work at a job and reports this as a separate domain.

### Study Endpoints

Study endpoints include:
Change from baseline in the weekly average of daily leg pain intensity on a numeric pain rating scale (NPRS) at Week 12 compared to placebo;
Change from baseline in the weekly average of the Daily Sleep Interference Scale (DSIS) at Week 12 compared to placebo;
Safety and tolerability based on treatment emergent adverse events (TEAEs), laboratory test results, vital signs, and ECGs;
PK parameter estimates of Compound 1 and its metabolite;
Proportion of subjects categorized as much improved or very much improved at Week 12 on the Patient Global Impression of Change assessment (PGIC) compared to placebo;
Proportion of subjects with ≥30% reduction from baseline in the weekly average of daily leg pain intensity on the NPRS at Week 12 compared to placebo;
Proportion of subjects with ≥50% reduction from baseline in the weekly average of daily leg pain intensity on the NPRS at Week 12 compared to placebo;
Proportion of subjects with ≥70% reduction from baseline in the weekly average of daily leg pain intensity on the NPRS at Week 12 compared to placebo;
Change from baseline in the Medical Outcomes Study 36 item Short-form Health Status Version 2 (SF-36v2) Physical Component Summary (PCS) score at Week 12 compared to placebo;
Change from baseline in the SF-36v2 Mental Component Summary (MCS) score at Week 12 compared to placebo;
Change from baseline in pain severity and pain interference indices on the Brief Pain Inventory—Short-form (BPI-SF) at Week 12 compared to placebo;
Change from baseline on the EuroQoL Questionnaire—5 Dimension—5 Level (EQ-5D-5L) health index at Week 12 compared to placebo;
Change from baseline on the Neuropathic Pain Symptom Inventory (NPSI) at Week 12 compared to placebo;
Change from baseline in the Hospital Anxiety and Depression Scale (HADS) at Week 12 compared to placebo; and
Change from baseline in the Oswestry Disability Index (ODI) at Week 12 compared to placebo; and
Change from baseline in overall work impairment on Work Productivity and Activity Impairment—Neuropathic Pain (WPAI-NP) at Week 12 compared to placebo.

### Efficacy and Statistical Analyses

Baseline Scores. Baseline values for the NPRS daily pain intensity scores are defined as the average score from Day −7 to Day −1. For ECGs, the baseline value iss defined as the average of the non-missing pretreatment measurements (triplicate) collected before the first dose of study drug. For all other variables, unless otherwise specified, baseline values are defined as the most recent non-missing measurement collected before the first dose of study drug. Change (absolute change) from baseline is calculated as [post-baseline value]−[baseline value].

Statistical Analysis. The primary efficacy analysis is based on a mixed effects model for repeated measures (MMRM), with change from baseline in weekly average of daily leg pain intensity on the NPRS as the dependent variable, and fixed effects of treatment group, week (categorical), treatment group-by-week interaction, and baseline weekly average of daily leg pain intensity.

### Example 13

### Alternative Preparation of (2R,3S,4S,5R)-4-[[3-(3,4-difluoro-2-methoxy-phenyl)-4,5-dimethyl-5-(trifluoromethyl)tetrahydrofuran-2-carbonyl]amino]pyridine-2-carboxamide (Compound 1)

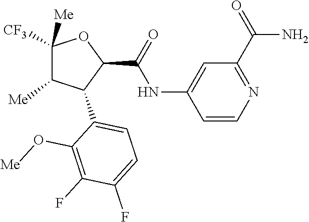 [SMILES: COc1c([C@H]2[C@H](C(=O)Nc3ccnc(C(N)=O)c3)O[C@@](C)(C(F)(F)F)[C@H]2C)ccc(F)c1F]

Intermediate compound 11 can be prepared by the following methods:

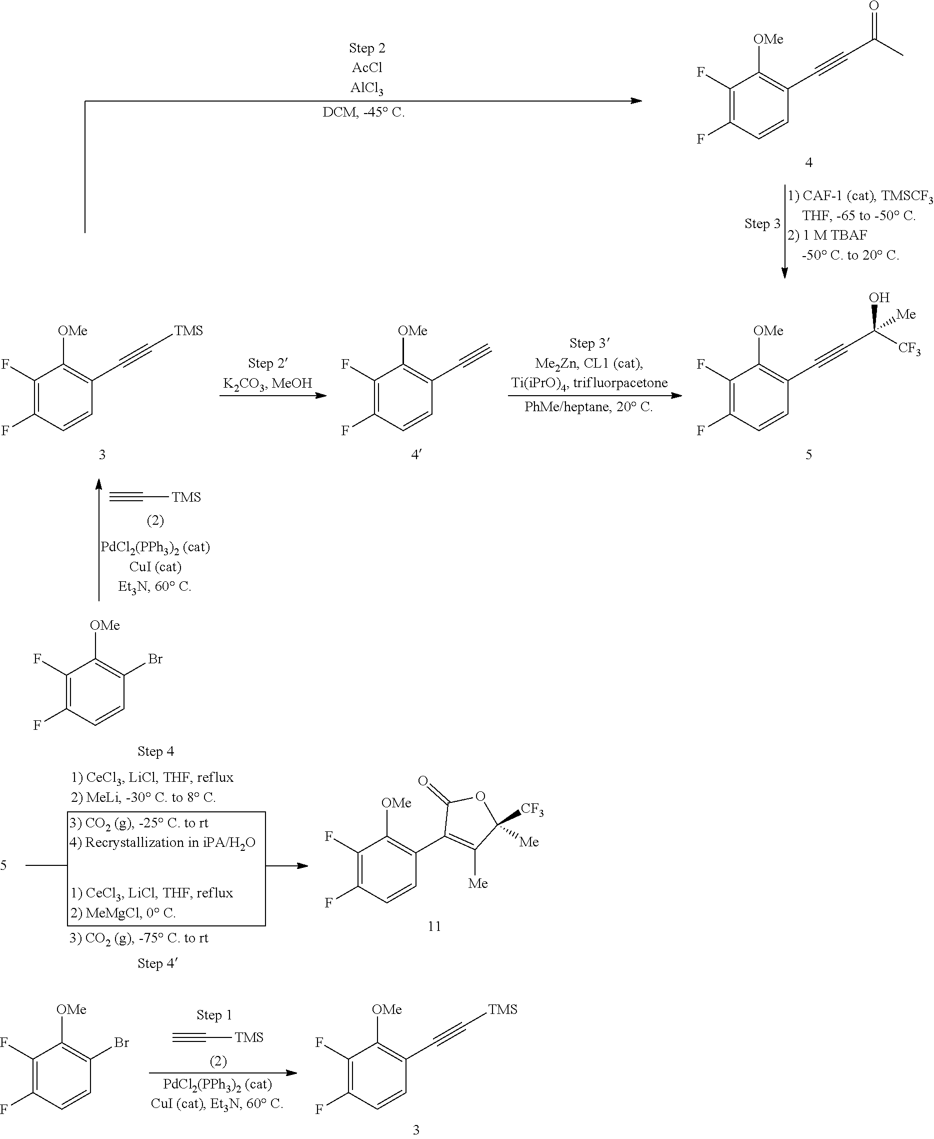

A 5.0 L three-neck round bottom flask equipped with a reflux condenser and a mechanical stirrer was charged with triethylamine (1.0 L, 5 vol). The triethylamine was sparged with nitrogen for 25 minutes. 1-Bromo-3,4-difluoro-2-methoxybenzene (200.1 g, 897.2 mmol) was charged under nitrogen atmosphere. The reaction mixture was heated to 50° C. (internal temp). Bis(triphenylphosphine)palladium(II) dichloride (15.8 g, 22.51 mmol) and copper(I) iodide (4.25 g, 22.31 mmol) were added. Trimethylsilylacetylene (2, 200.0 mL, 1.446 mol) was added over 100 minutes while maintaining the internal temperature of the reaction mixture below 72° C. The reaction mixture was stirred at 60° C. (internal temp) overnight. After 16 hours, more trimethylsilylacetylene (20.00 mL, 144.5 mmol) was added and the reaction mixture was stirred at 60° C. (internal temp). After 7 hours, the reaction mixture was cooled down and stirred at room temperature overnight (hold point). The reaction mixture was concentrated under vacuum to 532 g at 45° C. (down to 50 torr). Heptanes (1.0 L, 5 vol) were added to the residue and the mixture was concentrated under vacuum to 532 g at 45° C. (down to 50 torr). Heptanes (1.0 L, 5 vol) and activated charcoal (20 g, 10% by wt) were added and the mixture was stirred at 45° C. (external temp) for 40 minutes. The slurry was filtered over silica gel (300 g, 1.5 parts). The silica plug was then washed with heptanes (2×1.0 L, 10 vol). The filtrate was concentrated under vacuum at 45° C. (down to 18 torr) to provide [2-(3,4-difluoro-2-methoxyphenyl)ethynyl]trimethylsilane (3, 220.7 g, 89%) as a light yellow oil with 86.8% w/w purity by ¹H qNMR (ca. 6.9% w/w of heptanes) and 99.2% purity by HPLC (LCAP, 215 nm). ¹H NMR (400 MHz, CHLOROFORM-d) δ 7.18-7.10 (m, 1H), 6.87-6.74 (m, 1H), 4.06 (d, J=1.5 Hz, 3H), 0.27 (s, 9H). ¹⁹F NMR (377 MHz, CHLOROFORM-d) δ−133.07-−133.41 (m, 1F), −154.91 (br dd, J=19.8, 7.5 Hz, 1F). No mass detected by LC-MS.

### Step 2

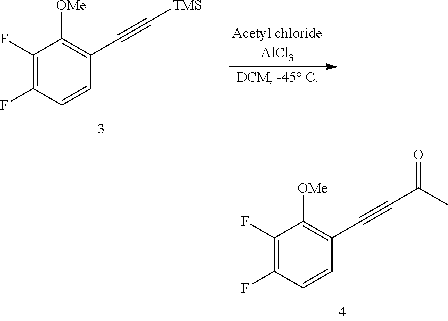 [SMILES: COc1c(C#C[Si](C)(C)C)ccc(F)c1F ; COc1c(C#CC(C)=O)ccc(F)c1F]

To a suspension of aluminium chloride (25.3 mL, 237 mmol) in dichloromethane (541 mL, 9.5 vol) at −45° C. under nitrogen atmosphere was added a solution of acetyl chloride (17.4 mL, 243 mmol) and [2-(3,4-difluoro-2-methoxyphenyl)ethynyl]trimethylsilane (3, 56.7 g, 205 mmol) in dichloromethane (97.2 mL, 1.7 vol) over 40 minutes (Tmax=−42° C.). After 30 minutes, 99% conversion was observed by HPLC (LCAP, 215 nm). The reaction mixture was poured into water (1 L, 17.6 vol) at 2° C. The phases were split and the aqueous layer was extracted with dichloromethane (100 mL, 1.8 vol). The organic layers were combined, washed with 15% aqueous sodium chloride (500 mL, 8.8 vol), dried over sodium sulfate (100 g), filtered, and concentrated under vacuum (40° C., 40 torr). The residue (50.2 g) was pre-absorbed on silica gel (75 g, 1.3 part) using dichloromethane. Purification by flash chromatography on silica gel (660 g, 11.6 parts) using 0-20% methyl tert-butyl ether in heptanes provided 4-(3,4-difluoro-2-methoxyphenyl)but-3-yn-2-one (4, 41.0 g, 93%) as a yellow oil with 97.9% w/w purity by 19F qNMR. 1H NMR (400 MHz, CHLOROFORM-d) δ 7.32-7.23 (m, 1H), 6.90 (td, J=9.1, 7.2 Hz, 1H), 4.15 (d, J=2.4 Hz, 3H), 2.49 (s, 3H). ¹⁹F NMR (377 MHz, CHLOROFORM-d) δ−128.81-−129.10 (m, 1F), −153.89-−154.18 (m, 1F). ESI-MS m/z calc. 210.1, found 211.1 (M+1)+; Retention time: 2.89 minutes.

### Step 3

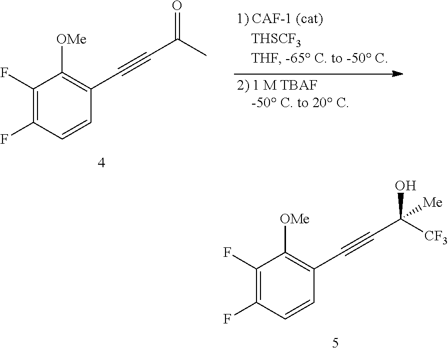 [SMILES: COc1c(C#CC(C)=O)ccc(F)c1F ; COc1c(C#C[C@@](C)(O)C(F)(F)F)ccc(F)c1F]

To a solution of (1S,2R,4S,5R)-1-{[3,5-bis(trifluoromethyl)phenyl]methyl}-5-ethenyl-2-[(S)-hydroxy(quinolin-4-yl)methyl]-1-azabicyclo[2.2.2]octan-1-ium fluoride (CAF-1, 2.34 g, 3.75 mmol) and 4-(3,4-difluoro-2-methoxyphenyl)but-3-yn-2-one (4, 41.2 g, 191 mmol) in THF (631 mL, 15.3 vol) at −65° C. was added dropwise trimethyl(trifluoromethyl)silane (57.1 mL, 386 mmol) over 15 minutes (Tmax=−61° C.) under nitrogen atmosphere. The reaction mixture was stirred at −60° C. for 3 hours then warmed up and stirred between −50 to −40° C. After 2 hours, 95% conversion was observed by HPLC (LCAP, 215 nm). A solution of TBAF in tetrahydrofuran (1.0 M, 195 mL, 195 mmol) was added over 15 minutes at −30° C. (Tmax=−21° C.). The reaction mixture was warmed up to room temperature. Heptanes (290 ml, 7 vol) and 5% aqueous citric acid (400 mL, 10 vol) were added. The phases were split, and the organic phase was washed with 5% aqueous citric acid (400 mL, 10 vol) and 15% aqueous sodium chloride (400 mL, 10 vol), dried over anhydrous sodium sulfate, filtered, and concentrated to afford crude material (56.8 g, 81.98% w/w content by 19F qNMR) as a yellow oil. The residue was dissolved in heptanes (75 mL, 1.8 vol) and purified by flash chromatography on silica gel (660 g, 16 parts; 0 to 20% of methyl tert-butyl ether in heptanes) to afford (2R)-4-(3,4-difluoro-2-methoxyphenyl)-1,1,1-trifluoro2-methylbut-3-yn-2-ol (5, 43.6 g, 75%) as a yellow oil with 59.9% ee (chiral GC). 1H NMR (400 MHz, CHLOROFORM-d) δ 7.14 (ddd, J=8.8, 5.7, 2.3 Hz, 1H), 6.85 (td, J=9.1, 7.2 Hz, 1H), 4.05 (d, J=1.7 Hz, 3H), 2.92 (s, 1H), 1.74 (s, 3H). 19F NMR (377 MHz, CHLOROFORM-d) δ−82.71 (s, 3F), −131.53-−131.71 (m, 1F), −154.16-−154.36 (m, 1F). ESI-MS m/z calc. 280.1, found 263.0 (M-17)+; Retention time: 3.08 minutes.

### Step 4

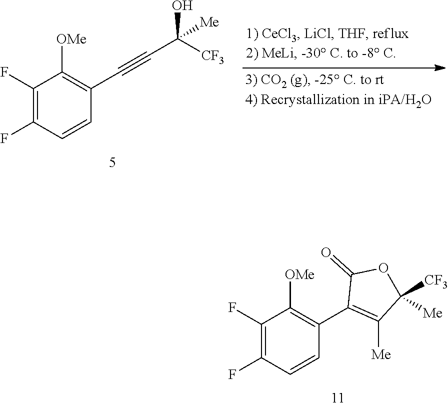 [SMILES: COc1c(C#C[C@@](C)(O)C(F)(F)F)ccc(F)c1F ; COc1c(C2=C(C)[C@](C)(C(F)(F)F)OC2=O)ccc(F)c1F]

A suspension of anhydrous cerium (III) chloride (71.7 g, 291 mmol) in a dry solution of lithium chloride in tetrahydrofuran (0.41 M, 1.47 L, 43 vol, 601 mmol) was heated at 68° C. for 2.5 hours. The beige hazy solution was cooled down to room temperature and a solution of 4-(3,4-difluoro-2-methoxyphenyl)-1,1,1-trifluoro-2-methylbut-3-yn-2-ol (5, 34.2 g, 118 mmol) in anhydrous tetrahydrofuran (67.0 mL, 2 vol) was added. The reaction mixture was cooled down to −30° C. and a solution of methyllithium in diethoxymethane (2.54 M, 258 mL, 655 mmol) was added over 25 minutes (Tmax=−21° C.). The reaction mixture was warmed up and stirred at −8° C. After 3.5 hours, 85% conversion was observed by HPLC (LCAP, 215 nm). The reaction mixture was cooled down to −25° C. and the argon inlet was replaced by a carbon dioxide inlet. The argon was slowly removed with a needle placed above the reaction mixture (Tmax=−19° C.) and the reaction mixture was warmed up to room temperature over 16 hours. Hydrochloric acid (1.5 M, 680 mL, 20 vol) and methyl tert-butyl ether (340 mL, 10 vol) were added. The biphasic mixture was stirred for 15 minutes at room temperature and the phases were split. The organic phase was washed with hydrochloric acid (1.5 M, 680 mL, 20 vol), with 10% aqueous potassium bicarbonate (680 mL, 20 vol), and with 15% aqueous sodium chloride (350 mL, 10 vol), dried over anhydrous sodium sulfate, filtered, concentrated, and co-evaporated with isopropanol (2×350 mL, 2×10 vol) under vacuum. The residue was dissolved in isopropanol (280 mL, 8.2 vol), and activated charcoal (3.5 g, 10% by wt.) was added. The slurry was stirred at 45° C. for 1 hour, ethyl acetate (68 mL, 2 vol) was added, and the slurry was filtered on Celite without cooling. The filter cake was washed with a mixture of isopropanol/ethyl acetate (4:1, 3×50 mL, 3×1.5 vol). The filtrate was concentrated and co-evaporated with isopropanol (2×270 mL, 2×10 vol), under vacuum. The resulting brown oil (41.32 g at 63.2% w/w content by 19F NMR; 17.7% w/w of isopropanol, 69% yield) was dissolved in isopropanol (64 mL, 2.46 vol; total volume of isopropanol=73 mL, 2.8 vol relative to the content in product) and the mixture was heated at 45° C. Water (52 mL, 2 vol relative to the content in product) was added over 15 minutes and the solution was cooled to 18° C. over 1 hour following a linear cooling profile. After stirring for 1 hour, the solids were filtered, washed with a mixture of isopropanol/water (1:1, 31 mL, 1.2 vol relative to the content in product), and dried under vacuum to afford (5R)-3-(3,4-difluoro-2-methoxyphenyl)-4,5-dimethyl-5-(trifluoromethyl)-2,5-dihydrofuran-2-one (11, 19.5 g, 76% recovery relative to the (R) enantiomer) as a light brown solid with 91.9% w/w purity by 19F qNMR and 76.5% ee by chiral GC. The material (19.5 g, 49.1 mmol) was dissolved in isopropanol (64 mL, 3.6 vol relative to the content in product), and the mixture was heated at 45° C. Water (30 mL, 1.7 vol relative to the content in product) was added over 1 hour, and the solution was cooled down to 18° C. over 3 hours following a linear cooling profile. After stirring for 1 hour, the solids were filtered, washed with a mixture of isopropanol/water (2:1, 2×18 mL, 2×1 vol relative to the content in product), and dried under vacuum to afford (5R)-3-(3,4-difluoro-2-methoxyphenyl)-4,5-dimethyl-5-(trifluoromethyl)-2,5-dihydrofuran-2-one (11, 12.8 g, 80% recovery relative to the (R) enantiomer) as a white solid with 99.9% purity by 19F qNMR and 96.7% ee by chiral GC. 1H NMR (400 MHz, CHLOROFORM-d) δ 7.02-6.93 (m, 2H), 3.92 (d, J=2.4 Hz, 3H), 2.05 (s, 3H), 1.76 (d, J=1.0 Hz, 3H). 19 F NMR (377 MHz, CHLOROFORM-d) δ −78.16 (s, 3F), −133.08-−133.82 (m, 1F), −153.36-−153.87 (m, 1F). ESI-MS m/z calc. 322.1, found 323.0 (M+1)+; Retention time: 3.26 minutes.

### Step 2′

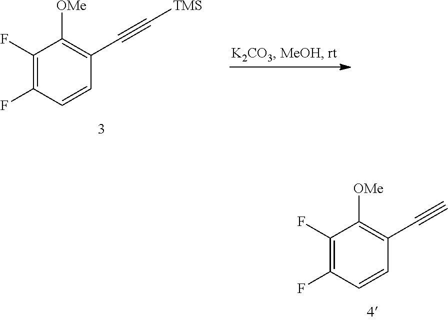 [SMILES: COc1c(C#C[Si](C)(C)C)ccc(F)c1F ; C#Cc1ccc(F)c(F)c1OC]

A 5.0 L three-neck round bottom flask under nitrogen atmosphere equipped with a mechanical stirrer was charged with [2-(3,4-difluoro-2-methoxyphenyl)ethynyl]trimethylsilane (3, 220.4 g, 86.8% w/w 796.0 mmol) and methanol (2.2 L, 10 vol). Potassium carbonate 325 mesh (142.0 g, 1.027 mol) was added over 15 minutes while keeping the internal temperature below 6° C. using a water/ice/sodium chloride bath. The reaction mixture was slowly warmed up to 14° C. over 1.5 hours after which full conversion was observed by HPLC (LCAP, 215 nm). The reaction mixture was filtered over Celite in a fritted funnel (600 mL, coarse). The filter cake was rinsed with methanol (450 mL, 2.0 vol). The filtrate was concentrated under vacuum (25° C., 30 torr). Water (600 mL, 2.7 vol) was added and the aqueous mixture was extracted with hexanes (500 mL then 300 mL, 3.6 vol). The organic layers were combined, washed with 15% aqueous sodium chloride (300 mL, 1.4 vol), dried over anhydrous sodium sulfate, filtered, and concentrated under vacuum (25° C., 30 torr) to provide 1-ethynyl-3,4-difluoro-2-methoxybenzene (4′, 133.3 g, 88%) as a light yellow oil with 88.4% w/w purity by ¹H qNMR (contains 4.1% w/w of hexanes) and 96.5% purity by HPLC (LCAP, 215 nm). ¹H NMR (400 MHz, CHLOROFORM-d) δ 7.21-7.12 (m, 1H), 6.88-6.77 (m, 1H), 4.07 (d, J=2.0 Hz, 3H), 3.28 (d, J=0.5 Hz, 1H). ¹⁹F NMR (377 MHz, CHLOROFORM-d) δ−132.65-−132.93 (m, 1F), −154.52-−154.80 (m, 1F). No mass detected by LC-MS.

### Step 3′

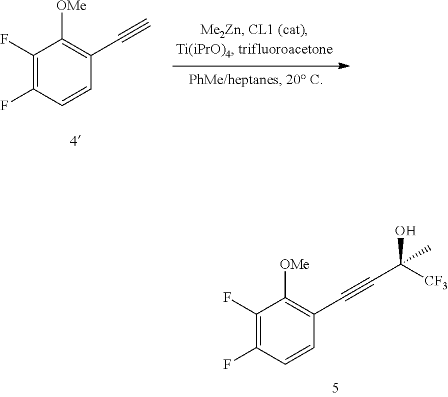 [SMILES: C#Cc1ccc(F)c(F)c1OC ; COc1c(C#C[C@@](C)(O)C(F)(F)F)ccc(F)c1F]

To a solution of 1-((1S,2R,4R)-2-hydroxy-7,7-dimethylbicyclo[2.2.1]heptan-1-yl)-N-((1R,2R)-2-((((1S,2R,4R)-2-hydroxy-7,7-dimethylbicyclo[2.2.1]heptan-1-yl)methyl)sulfonamido)cyclohexyl)methanesulfonamide (CL1, 18.3 g, 77.2% w/w, 26.2 mmol) and titanium (IV) isopropoxide (116 mL, 393 mmol) in anhydrous heptanes (454 mL, 9.1 vol) was added a 1.03 M solution of dimethylzinc in toluene (422 mL, 435 mmol) over 2 minutes at 20° C. The reaction mixture was stirred at room temperature for 10 minutes and a solution of 1-ethynyl-3,4-difluoro-2-methoxy-benzene (4′, 50.0 g, 88.1% w/w, 262 mmol) in anhydrous heptanes (136 mL, 2.7 vol) was added at 20° C. The reaction mixture was stirred at room temperature for 1 hour and 1,1,1-trifluoroacetone (35.2 mL, 393 mmol) was added at 20° C. The reaction mixture was stirred at room temperature for 17 hours and more of the 1.03 M solution of dimethylzinc in toluene (64.0 mL, 66.0 mmol) followed by more of the 1,1,1-trifluoroacetone (5.91 mL, 66.0 mmol) were added. The reaction mixture was stirred at room temperature for 7 hours and more of the 1.03 M solution of dimethylzinc in toluene (64.0 mL, 66.0 mmol) followed by more of the 1,1,1-trifluoroacetone (5.91 mL, 66.0 mmol) were added. The reaction mixture was stirred at room temperature for 17 hours and more 1.03 M solution of dimethylzinc in toluene (64.0 mL, 66.0 mmol) followed by more 1,1,1-trifluoroacetone (5.91 mL, 66.0 mmol) were added. The reaction mixture was stirred at room temperature for 8 hours and more of the 1.03 M solution of dimethylzinc in toluene (64.0 mL, 66.0 mmol) followed by more of the 1,1,1-trifluoroacetone (5.91 mL, 66.0 mmol) were added. The reaction mixture was stirred at room temperature for 16 hours and more of the 1.03 M solution of dimethylzinc in toluene (64.0 mL, 66.0 mmol) followed by more of the 1,1,1-trifluoroacetone (5.91 mL, 66.0 mmol) were added. The reaction mixture was stirred at room temperature for 24 hours, cooled down to 8° C. and 6 M hydrochloric acid (500 mL, 10 vol) was added over 15 minutes (Tmax=17° C.). The heterogenous mixture was stirred at room temperature for 1.5 hours, and the phases were split. The organic phase was washed with 3 M hydrochloric acid (500 mL, 10 vol). The organic phase was concentrated, and toluene (250 mL, 5 vol) and heptanes (500 mL, 10 vol) were added. The organic phase was washed with 18% w/w aqueous sodium chloride (500 mL, 10 vol), and the biphasic mixture was stirred at room temperature for 0.75 hour. The resulting slurry/emulsion was filtered, and the white solid was washed with heptanes (100 mL, 2 vol) and dried under vacuum to afford 1-((1S,2R,4R)-2-hydroxy-7,7-dimethylbicyclo[2.2.1]heptan-1-yl)-N-((1R,2R)-2-((((1S,2R,4R)-2-hydroxy-7,7-dimethylbicyclo[2.2.1]heptan-1-yl)methyl)sulfonamido)cyclohexyl)-methanesulfonamide (CL1, 13.4 g, 71% recovery) as a white solid. The phases from the filtrate were split and the organic phase was dried over sodium sulfate, filtered, and concentrated (14 torr, 45° C.). The resulting yellow oil was diluted in heptanes (100 mL, 2 vol) and purified by flash chromatography (330 g of SiO₂; 0 to 10% of methyl tert-butyl ether in heptanes) to afford (2R)-4-(3,4-difluoro-2-methoxyphenyl)-1,1,1-trifluoro-2-methylbut-3-yn-2-ol (5, 70.6 g, 86%) as a colorless oil with 93% ee by chiral SFC. ¹H NMR (400 MHz, CHLOROFORM-d) δ 7.14 (ddd, J=8.8, 5.7, 2.3 Hz, 1H), 6.85 (td, J=9.1, 7.2 Hz, 1H), 4.05 (d, J=1.7 Hz, 3H), 2.94 (brs, 1H), 1.74 (d, J=1.0 Hz, 3H). ¹⁹F NMR (377 MHz, CHLOROFORM-d) δ−82.71 (s, 3F), −131.57-−131.77 (m, 1F), −154.16-−154.41 (m, 1F). ESI-MS m/z calc. 280.1, found 263.0 (M-17)f; Retention time: 3.04 minutes.

### Step 4′

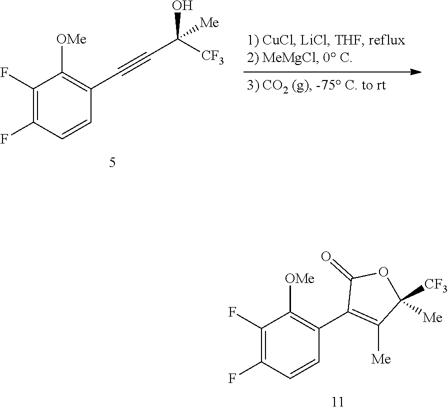 [SMILES: COc1c(C#C[C@@](C)(O)C(F)(F)F)ccc(F)c1F ; COc1c(C2=C(C)[C@](C)(C(F)(F)F)OC2=O)ccc(F)c1F]

A suspension of copper (I) chloride (18.7 g, 189 mmol) in a 0.4 M solution of lithium chloride in tetrahydrofuran (1.02 L, 409 mmol) was heated at 67° C. under mechanical stirring for 3 hours. A hazy solution with some residual solids in suspension was obtained. The mixture was cooled down to 2° C., and a 2.98 M solution of methylmagnesium chloride in tetrahydrofuran (251 mL, 747 mmol) was added over 20 minutes (Tmax=7° C.). The reaction mixture was stirred at 0° C. for 30 minutes, and a solution of (2R)-4-(3,4-difluoro-2-methoxyphenyl)-1,1,1-trifluoro-2-methylbut-3-yn-2-ol (5, 51.5 g, 92.6% w/w, 93% ee, 164 mmol) in tetrahydrofuran (46.0 mL, 0.9 vol) was added over 15 minute (Tmax=9° C.). The reaction mixture was warmed up and stirred at 30° C. for 18 hours. Then, the reaction mixture was cooled down to −73° C. and the argon inlet was replaced by carbon dioxide (Tmax=−58° C.). The reaction mixture was warmed up to room temperature over 4 hours, and 3 M hydrochloric acid (500 mL, 9.7 vol) was added over 20 minutes (Tmax=27° C., cooling with an ice bath to control the exotherm). The heterogeneous mixture was stirred at 24° C. for 1 hour. The organic phase was washed with 10% w/w aqueous KHCO₃ (500 mL, 9.7 vol). The phases were split, the organic phase was washed with 10% w/w aqueous KHCO₃ (500 mL, 9.7 vol), and the resulting emulsion was filtered on Celite. The phases were split, and the basic aqueous washes were combined and extracted with methyl tert-butyl ether (200 mL, 3.9 vol). The phases were split, and the organic phase was washed with 10% w/w aqueous KHCO₃ (200 mL, 3.9 vol). The resulting emulsion was filtered on the same Celite cake used above. The filter cake was washed twice with methyl tert-butyl ether (2×100 mL, 2×1.9 vol). The phases from the filtrate were split and the organic phase was combined with the previous organic phase. The combined organic phase was washed with 20% w/w aqueous NH₄Cl (500 mL, 9.7 vol) and with 15% w/w aqueous NaCl (500 mL, 9.7 vol), dried over sodium sulfate, filtered, and concentrated under vacuum to afford crude material containing 38.9 g (74% chemical yield) of (5R)-3-(3,4-difluoro-2-methoxyphenyl)-4,5-dimethyl-5-(trifluoromethyl)-2,5-dihydrofuran-2-one (11) by ¹⁹F qNMR. The crude material was dissolved in isopropanol (500 mL, 12.9 vol relative to the content in 11), and the solvent was evaporated under reduced pressure. The residue was dissolved in isopropanol (416 mL, 10.7 vol relative to the content in 11), and activated charcoal (5 g, 13% by wt. rel. to the content in 11) was added. The slurry was heated and stirred at 45° C. for 1 hour. Ethyl Acetate (104 mL, 2.7 vol relative to the content in 11) was added, and the slurry was filtered on Celite. The filter cake was rinsed three times with a mixture of isopropanol/ethyl acetate (4:1, 3×75 mL, 3×1.9 vol relative to the content in 11). The filtrate was concentrated and co-evaporated with isopropanol (2×500 mL, 2×12.9 vol relative to the content in 11), and the residue was dissolved in isopropanol (113 mL, 2.9 vol relative to the content in 11) at 45° C. Water (81 mL, 2.1 vol relative to the content in 11) was added over 1 hour with a peristaltic pump. The solution was seeded with 50 mg of pure (R)-enantiomer of 11 after 25% of the addition of the water was completed. The resulting slurry was cooled down to room temperature over 1 hour following a linear cooling profile and stirred at room temperature for 1 hour. The solids were filtered, washed with a mixture of isopropanol/water (1:1, 48 mL, 1.2 vol relative to the content in 11) and dried under vacuum to afford (5R)-3-(3,4-difluoro-2-methoxyphenyl)-4,5-dimethyl-5-(trifluoromethyl)-2,5-dihydrofuran-2-one (11, 39.1 g, 71% for the (R)-enantiomer) as a white solid with 97.8% w/w purity by ¹⁹F qNMR and 98.5% ee by chiral GC. ¹H NMR (400 MHz, CHLOROFORM-d) δ 7.01-6.92 (m, 2H), 3.92 (d, J=2.2 Hz, 3H), 2.05 (d, J=0.7 Hz, 3H), 1.76 (d, J=0.7 Hz, 3H). ¹⁹F NMR (377 MHz, CHLOROFORM-d) δ−78.16 (s, 3F), −133.26-−133.57 (m, 1F), −153.51-−153.79 (m, 1F). ESI-MS m/z calc. 322.1, found 323.0 (M+1)f; Retention time: 3.24 minutes.

### Synthesis of Catalyst CAF-1 (Catalyst for Step 3)

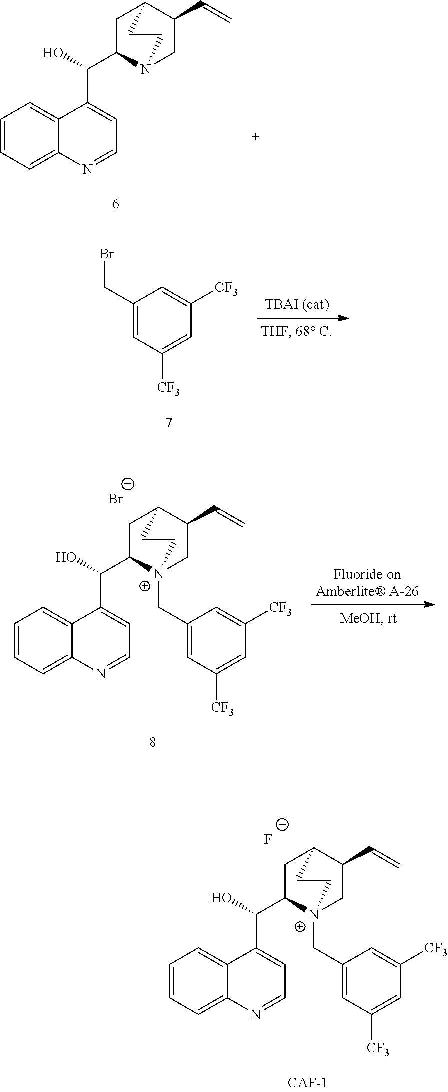

### (Ref: Caron, S.; Do, N. M.; Arpin, P.; Larivde, A. Synthesis 2003, 1693-1698)

To a suspension of (+)-cinchonine (6, 3.00 g, 10.2 mmol) in tetrahydrofuran (45.0 mL, 15 vol) under nitrogen atmosphere was added 3,5-bis(trifluoromethyl)benzyl bromide (7, 2.24 mL, 12.2 mmol) and tetrabutylammonium iodide (113 mg, 305 umol). The reaction mixture was stirred at 70° C. After 20 hours, 98% conversion was observed by HPLC (LCAP, 215 nm). Tetrahydrofuran (105 mL, 35 vol) was added. The reaction mixture was stirred 3 hours at 70° C. and cooled to room temperature. The reddish suspension was filtered. The solids were rinsed twice with tetrahydrofuran (2×50 mL, 2×17 vol) and dried under vacuum to afford (1S,2R,4S,5R)-1-{[3,5-bis(trifluoromethyl)phenyl]methyl}-5-ethenyl-2-[(S)-hydroxy(quinolin-4-yl)methyl]-1-azabicyclo[2.2.2]octan-1-ium bromide (8, 5.9 g, 90%) as a white solid with 93.1% purity (LCAP, 215 nm). A sample of the material obtained above (2.56 g, 4.2 mmol) was dissolved in MeOH (25.0 mL, 10 vol), and freshly washed fluoride on polymer support (2.50 g, 6.25 mmol) was added. The slurry was stirred at room temperature for 3 hours. Completion of the reaction was confirmed by 19F NMR (ca. 98% conversion). The mixture was filtered and the resin was rinsed twice with tetrahydrofuran (2×5 mL, 2×2 vol). The filtrate was concentrated and coevaporated with toluene (3×25 mL, 3×10 vol) to afford (1S,2R,4S,5R)-1-{[3,5-bis(trifluoromethyl)phenyl]-methyl}-5-ethenyl-2-[(S)-hydroxy(quinolin-4-yl)methyl]-1-azabicyclo[2.2.2]octan-1-ium fluoride (CAF-1, 2.17 g, 93%) as a white solid with 93.3% purity (LCAP, 215 nm). 1H NMR (400 MHz, DMSO-d6) δ 13.68 (br s, 1H), 8.89 (br s, 1H), 8.45 (br s, 2H), 8.32 (br s, 1H), 8.28-8.18 (m, 1H), 8.13-7.96 (m, 1H), 7.84 (d, J=4.4 Hz, 1H), 7.82-7.74 (m, 1H), 7.74-7.64 (m, 1H), 6.40-6.16 (m, 2H), 6.03 (ddd, J=17.4, 10.3, 7.3 Hz, 1H), 5.27-5.13 (m, 2H), 5.13-4.95 (m, 1H), 4.67 (br s, 1H), 3.89 (br s, 1H), 3.71 (br t, J=9.2 Hz, 1H), 3.33-3.21 (m, 1H), 3.13-2.99 (m, 1H), 2.58-2.52 (m, 1H), 2.41-2.19 (m, 1H), 1.86-1.67 (m, 3H), 0.94-0.84 (m, 1H). 19F NMR (377 MHz, DMSO-d6) δ−61.09 (s, 6F), −112.28 (br s, 1F). ESI-MS m/z calc. 521.2, found 521.2 (M)+; Retention time: 2.93 minutes.

### Synthesis of Catalyst CL1 (Catalyst for Step 3′)

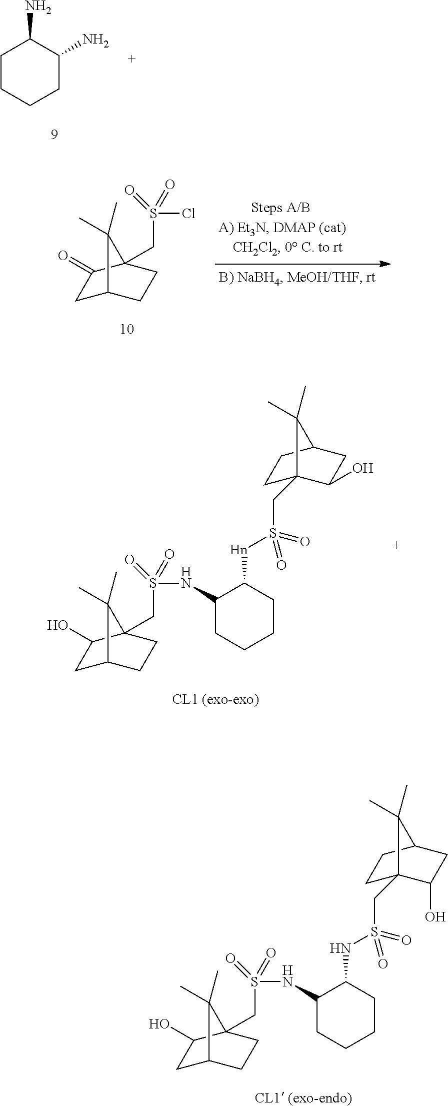

(Ref: Forrat, V. J.; Prieto, O.; Ramon, D. J.; Yus, M. Chem. Eur. J. 2006, 12, 4431-4445.)

A solution of (1R,2R)-(−)-1,2-diaminocyclohexane (9, 16.5 g, 144 mmol) in dichloromethane (851 mL, 52 vol) was charged under nitrogen atmosphere in a 3.0 L jacketed Chemglass reactor equipped with a mechanical stirred and a chiller (glycol/water, 1:1). Triethylamine (43.1 mL, 309 mmol) and 4-dimethylaminopyridine (8.12 g, 66.5 mmol) were added at room temperature. The reaction mixture was cooled down to 0° C. and a solution of (1S)-(+)-10-camphorsulfonyl chloride (10, 76.1 g, 303 mmol) in dichloromethane (851 mL, 52 vol) was added over 20 minutes (Tmax=5.2° C.). After 60 minutes, the reaction mixture was warmed up and stirred at room temperature overnight (hold point).

A 1.5 M aqueous solution of sodium hydroxide (200 mL, 12 vol) was added. The organic layer was separated and washed with a mixture of 1.5 M aqueous sodium hydroxide (100 mL, 6 vol) and 15% aqueous sodium chloride (100 mL, 6 vol). The basic washes were combined and extracted with dichloromethane (100 mL, 6 vol). The combined organic layers were washed with 1.5 M hydrochloric acid (250 mL, 15 vol, exotherm=18 to 21° C.) and with 15% aqueous sodium chloride (200 mL, 12 vol), dried over anhydrous sodium sulfate (400 g), filtered, and concentrated under vacuum to provide 71.0 g of crude material (diketone) as a beige solid.

The material obtained above still contained traces of 4-dimethylaminopyridine by ¹H NMR. Therefore, it was dissolved in dichloromethane (500 mL, 30 vol) at 35° C. The organic solution was then washed with 1.5 M hydrochloric acid (2×200 mL, 24 vol), with 15% aqueous sodium chloride (200 mL, 12 vol), dried over anhydrous sodium sulfate (150 g), filtered, and concentrated under vacuum to provide 66.1 g of crude material (diketone) as a beige solid showing no more traces of 4-dimethylaminopyridine by ¹H NMR.

The material obtained above (66.1 g) was dissolved in tetrahydrofuran (850 mL, 13 vol rel. to diketone) at 35° C., and then methanol (1.15 L, 17 vol rel. to diketone) was added. The reaction mixture was cooled down to 16.5° C. and sodium borohydride (31.2 g, 825 mmol) was added portionwise over 20 minutes while keeping the internal temperature below 20° C. The reaction mixture was then stirred at room temperature overnight (hold point).

The reaction mixture was quenched with 20% aqueous ammonium chloride (750 mL, 11 vol rel. to diketone) at room temperature. The aqueous mixture was concentrated (not to dryness) under vacuum (down to 65 torr at 40° C.). The aqueous mixture was extracted with ethyl acetate (700 mL, 11 vol rel. to diketone). The organic layer was separated, washed with water (300 mL, 4.5 vol rel. to diketone), with 15% aqueous sodium chloride (300 mL, 4.5 vol rel. to diketone), dried over anhydrous sodium sulfate (175 g), filtered, and concentrated to 186 g under vacuum at 40° C. Toluene (325 mL, 4.9 vol rel. to diketone) was added and the mixture was concentrated to 360 g under vacuum at 40° C. Seed crystals (CL1) were added (spontaneous crystallization was observed). The mixture was stirred from 45 to 25° C. (external temp) over 1 hour. The solids were collected in a Bichner funnel, rinsed with toluene (150 mL, 2.3 vol rel. to diketone), and dried under high vacuum (1.8 torr) at 50° C. to provide 1-((1S,2R,4R)-2-hydroxy-7,7-dimethylbicyclo[2.2.1]heptan-1-yl)-N-((1R,2R)-2-((((1S,2R,4R)-2-hydroxy-7,7-dimethylbicyclo[2.2.1]heptan-1-yl)methyl)sulfonamido)cyclohexyl)methanesulfonamide (CL1, 51.7 g, 51%, 2 steps) as a white solid with 77.2% w/w purity (12.9% w/w of the exo-endo diastereoisomer, 8.2% w/w of toluene) by ¹H qNMR. ¹H NMR (400 MHz, CHLOROFORM-d) δ 5.37 (br d, J=7.3 Hz, 2H), 4.10 (dt, J=7.8, 4.1 Hz, 2H), 3.53-3.45 (m, 4H), 3.14-3.03 (m, 2H), 2.91 (d, J=13.7 Hz, 2H), 2.15 (br d, J=12.0 Hz, 2H), 1.82-1.74 (m, 12H), 1.52 (br t, J=9.5 Hz, 2H), 1.44-1.31 (m, 4H), 1.17-1.10 (m, 2H), 1.07 (s, 6H), 0.84 (s, 6H). ESI-MS m/z calc. 546.28, found 545.2 [M-1]-; Retention time: 3.478 minutes.

Intermediate compound 11 can be transformed to Compound 1 by the methods described in Examples 1, 8, and 10 of International Publication No. WO 2024/123815, which is incorporated by reference. For completeness, the substance of those Examples is reproduced below.

Compound 17 can be prepared from Compound 11 by the process described below:

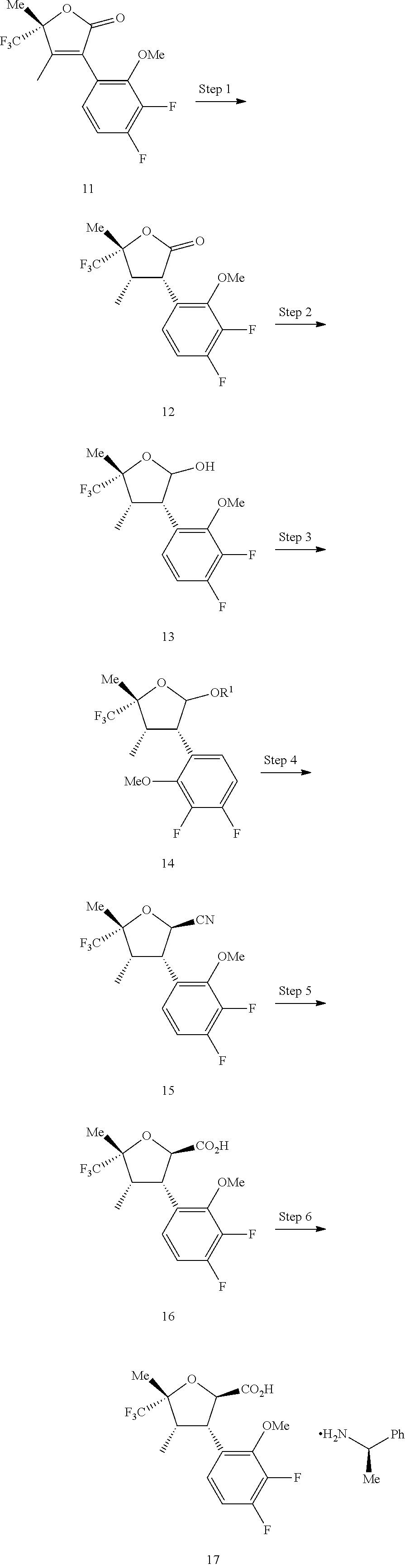

In some embodiments, R¹ is —C(O)CH₃, as described below in Steps 3A and 4A. In some embodiments, R¹ is —C(O)(4-nitrophenyl), as described below in Steps 3B and 4B.

### Step 1: Synthesis of (3S,4S,5R)-3-(3,4-difluoro-2-methoxyphenyl)-4,5-dimethyl-5-(trifluoromethyl)dihydrofuran-2(3H)-one (12)

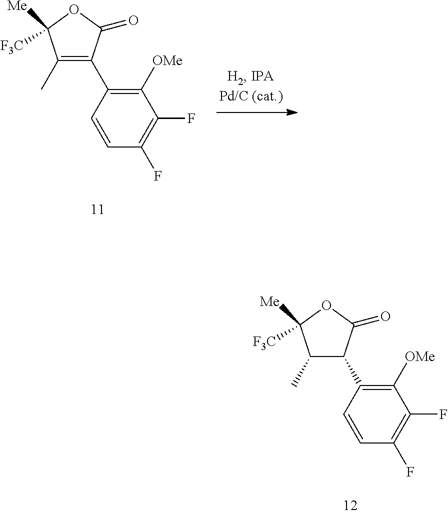 [SMILES: COc1c(C2=C(C)[C@](C)(C(F)(F)F)OC2=O)ccc(F)c1F ; COc1c([C@H]2C(=O)O[C@@](C)(C(F)(F)F)[C@H]2C)ccc(F)c1F]

### Procedure 1:

Compound 11, obtained from Step 1 above (1200 g, 3.72 mol), was added to the 11L hydrogenation reactor (previously flushed with N₂ gas and set for a slow N₂ gas sweep). The catalyst (Pd/C, e.g., Johnson Matthey Catalog No. A503032-5 or A503014-5, 1200 g, 4.89% Pd, 63.8% water, corresponding to 21.2 g Pd, 0.0536 molar eq.) was added. Isopropanol (7.0 L) was added. The reactor was sealed, purged 3×N₂ gas/vacuum, then with 50 psi nitrogen/hydrogen, and finally adjusted to 225 psi hydrogen. The jacket was set to 30-31° C. and agitation was started. Agitation was continued for 30 hours; an NMR sample (0.2 ml+2.0 ml MTBE+1.0 ml of 5% KHCO₃; evaporation of 1.5 ml of the MTBE, and NMR); showed 2.8% starting material. Reaction was considered complete. The slurry was filtered (solka-floc) and washed with 10 L of IPA, then 3 L DCM. The filtrate was concentrated to an oil, and redissolved in 3 L of toluene. The toluene solution was re-concentrated to an oil, 1152 g, 3.553 mols, 95.5% yield. Proton NMR of the product was consistent with compound 12.

¹H-NMR CDCl₃: δ 6.93-6.80 (m, 2H); 4.48 (d, 1H, J=9.5 Hz); 4.03 (d, 3H, J=3.1 Hz); 2.89 (dq, 1 H, J=9.5, 7.5 Hz); 1.71 (d, 3H, J=1.2 Hz); 0.84-0.76 (m, 3H) ppm.

### Procedure 2:

To a reactor rated for hydrogen and pressure service, and equipped with a gas dosing unit and pressure controller, was charged Compound 11 (1 equiv, limiting reagent), 5% palladium on carbon (0.05 equiv, e.g., Johnson Matthey Catalog No. A503032-5 or A503014-5, corrected for water content and palladium assay), tetrahydrofuran (1.75 volumes), 2-propanol (5.25 volumes), and trifluoroacetic acid (0.05 equiv). The vessel was pressurized to 3 barg with nitrogen, and then vented to ambient pressure. This sequence was performed 3 times. The reactor contents were adjusted to 30° C. Alternatively, the reactor contents can be adjusted to 50° C. The vessel was then pressurized to 3 barg with hydrogen, and vented to ambient pressure. This sequence was performed 3 times. The reactor was then pressurized to operating pressure (40 barg) with hydrogen, and agitation was started at a sufficient speed to achieve gassing of the liquid from the head space.

The reaction mixture was stirred at these conditions until reaction completion (Less than 1% Compound 11 and its diastereomers by GC).

The hydrogen in the headspace was vented. The reactor was pressurized to 3 barg with nitrogen and the reactor was vented. This sequence was performed 3 times. Cyclohexene (0.2 volumes) was charged to the reactor, and the reaction was maintained at 30° C. with stirring under nitrogen for no less than 15 minutes.

The reaction mixture was filtered over a bed of diatomaceous earth to remove the catalyst. The filter cake was washed with 2-propanol (4 volumes). The filtrates from the primary filtration and the wash were combined.

In a well mixed vessel, the filtrate was concentrated under reduced pressure at no more than 40° C. to a total of 3 volumes. Toluene (7 volumes) was charged and distillation was resumed under reduced pressure at no more than 50° C. until reaching a total of 3 volumes. Toluene (7 volumes) was charged and distillation was resumed under reduced pressure at no more than 50° C. until reaching a total of 3 volumes. Toluene (5 volumes) was charged and the solution was mixed well. Proton NMR of the product was consistent with compound 12.

¹H-NMR CDCl₃: δ 6.93-6.80 (m, 2H); 4.48 (d, 1H, J=9.5 Hz); 4.03 (d, 3H, J=3.1 Hz); 2.89 (dq, 1 H, J=9.5, 7.5 Hz); 1.71 (d, 3H, J=1.2 Hz); 0.84-0.76 (m, 3H) ppm.

### Step 2: Synthesis of (2S,3S,45,5R)-3-(3,4-difluoro-2-methoxyphenyl)-4,5-dimethyl-5-(trifluoromethyl)tetrahydrofuran-2-ol (13)

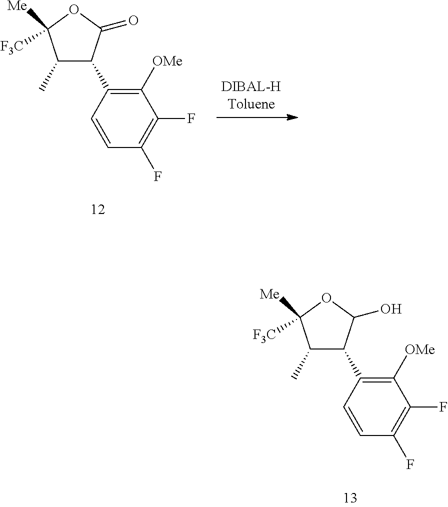 [SMILES: COc1c([C@H]2C(=O)O[C@@](C)(C(F)(F)F)[C@H]2C)ccc(F)c1F ; COc1c([C@H]2C(O)O[C@@](C)(C(F)(F)F)[C@H]2C)ccc(F)c1F]

### Procedure 1:

The product of Step 2 above (compound 12, 1052 g, 3.2445 mols) was added to a 50L jacketed reactor, and added 8.82 L of anhydrous toluene under N₂ gas. The resulting solution was cooled to −31° C. overnight under N₂ gas. Diisobutylaluminum hydride (1.96 kg of 25% solution in toluene, 3.445 mol, 1.056 eq) was slowly transferred to the reaction vessel through an addition funnel under N₂ gas. The hydride reagent was added to the reaction solution over 2 hours and the reactor temperature went from −31.6° to −27.4° C. during the addition. The solution was stirred at −26 to −27° C. for 90 minutes. A solution of 2.75 kg of potassium/sodium tartrate in 20 L of DI water was added over 2.5 hours. The reaction mixture was allowed to warm up until it reached 0° C., the cooler was turned to +24° C. after about 2 hours of addition. Toluene (5L) was added and the mixture was stirred overnight at +20° C.

The reaction mixture was transferred to a separatory funnel and separated aqueous phase from the organic phase. The aqueous phase was re-extracted with 5 L of toluene. The two toluene solutions were combined, treated with magnesium sulfate, and filtered. The solid was washed with toluene, and the combined toluene solution was vacuum concentrated to an oil, wt. 1055 g, 3.2336 mol, 99.7% of crude lactol. The crude product was directly taken into the next step (acetylation is exemplified below). The proton NMR of the crude material was consistent with compound 13.

¹H-NMR CDCl₃: δ 7.30-7.26 (m, 1H); 7.20-7.18 (m, 1H); 5.81 (d, 1H, J=4 Hz); 4.00 (s, 3 H); 3.84-3.80 (m, 1H); 2.92-2.88 (m, 1H); 1.67 (s, 3H); 0.83 (d, 3H, J=8 Hz) ppm.

### Procedure 2:

A solution of Compound 12 in toluene (about 8 vol) was added to a reactor. Alternatively, the compound of formula 12 can be added to the reactor with a target of 5 vol. Agitation was begun and mixture was cooled to −25+5° C. A solution of diisobutylaluminum hydride (25% w/w toluene) was added to the reaction mixture while maintaining the batch temperature at no more than −20° C. Temperature was adjusted to −25±5° C. and the batch was stirred for no less than one hour. Upon reaction completion, a solution of acetone in toluene (0.3 equiv in 0.5 vol) was added to the reaction mixture, maintaining temperature at −25±5° C. and stirred for no less than 30 minutes. Reaction mixture was then warmed to 0±5° C. A 12 vol solution of 0.62 M citric acid was transferred to the reactor while maintaining the temperature at 20±5° C. and the biphasic mixture was stirred for no less than four hours. Alternatively, 8 vol of a 0.62 M citric acid can be added to the reactor while maintaining the temperature at 20±5° C. and the biphasic mixture can be stirred for no less than four hours. Phases were allowed to settle and the bottom, aqueous phase was drained off. A 12-vol solution of 0.62 M citric acid was added to the batch and the biphasic mixture was stirred at 20±5° C. for no less than 30 minutes. Alternatively, 8 vol of a 0.62 M citric acid can be added to the batch and the biphasic mixture can be stirred at 20±5° C. for no less than 30 minutes. Phases were allowed to settle and the bottom, aqueous layer was drained off. 5 vol of water was charged to the batch and the biphasic mixture was stirred at 20±5° C. for no less than 30 minutes. Phases were allowed to settle and the bottom, aqueous layer was drained off. The organic layer was distilled to a total of 5 vol while maintaining an internal temperature at or below 45° C. 5 vol of toluene was charged and the mixture was distilled to a total of 5 vol. Distillation continued until residual water was below 0.1%. The proton NMR of the isolated material was consistent with compound 13.

¹H-NMR CDCl₃: δ 7.30-7.26 (m, 1H); 7.20-7.18 (m, 1H); 5.81 (d, 1H, J=4 Hz); 4.00 (s, 3 H); 3.84-3.80 (m, 1H); 2.92-2.88 (m, 1H); 1.67 (s, 3H); 0.83 (d, 3H, J=8 Hz) ppm.

### Step 3A: Synthesis of (3S,4S,5R)-3-(3,4-difluoro-2-methoxyphenyl)-4,5-dimethyl-5-(trifluoromethyl)tetrahydrofuran-2-yl acetate (14-B)

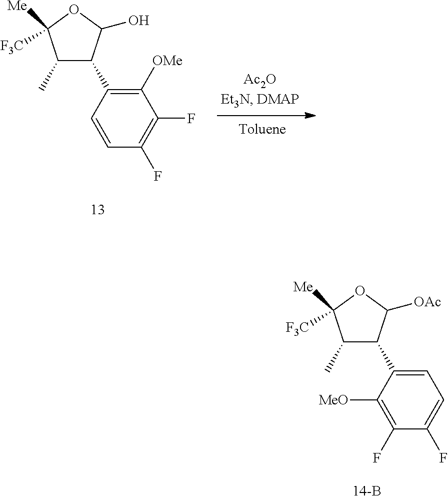 [SMILES: COc1c([C@H]2C(O)O[C@@](C)(C(F)(F)F)[C@H]2C)ccc(F)c1F ; COc1c([C@H]2C(OC(C)=O)O[C@@](C)(C(F)(F)F)[C@H]2C)ccc(F)c1F]

The crude product of Step 3 above (1055 g of compound 13, 3.23 mols) was transferred to a 25 L jacketed reactor pre-dried and kept under N₂ gas. Crude compound 13 was rinsed in with anhydrous toluene, 6.8 L and the mixture stirred to ensure complete solution at 20° C. Triethylamine (466 ml, 3.343 mols, 1.04 eq) was added, followed by DMAP (3.58 g, 0.0293 mols, 0.01 eq). Acetic anhydride, (313 ml, 3.288 mols, 1.02 eq) was added over about 5-10 minutes using an addition funnel (T rose from 21.1° C. to a T=26.4° C. during addition of acetic anhydride). The reaction mixture was stirred at 25° C. for 100 minutes. Proton NMR of the reaction mixture indicated that the reaction was complete at this point.

The reaction mixture was extracted with 3.5 L of 25% ammonium chloride, then 1200 ml of 10% KHCO₃. The aqueous layers were re-extracted with 2 L of toluene and the combined toluene solution was dried over magnesium sulfate, filtered and washed the filtered solid with toluene. The resulting toluene solution was concentrated to dryness, initially with a 20 L RB flask and then using a 3 L jacketed reactor (1898 g transferred) for the final concentration. The solution was vacuum-distilling to a thick oil, placed it on high-vacuum at 25° C. with stirring until the oil converts to a crystalline mass (with a small amount of a viscous oil remaining). Hexane (900 ml) was added to the reaction vessel (containing solid and some remnant oil) and stirred the mixture at 20° C. overnight. The mixture was cooled to 10° C. and stirred for 2 hours, then cooled to 4° C. while stirring for 6 hours, before cooling the mixture to −10° C. overnight (while stirring), and finally to −14° C. over the weekend (36 to 48 hours) to obtain a crystal slurry. The slurry was filtered with a jacketed filter at −15° C., and then washed the solid with cold (−16° C.) hexane (2×150 ml, then 100 ml). The crystals were dried at RT under vacuum. The resulting solid was dissolved in toluene (total 1450 ml solution) and assayed to obtain 800.2 g in solution.

Additional product (that was stuck to the reactor) was dissolved off with toluene to give 425 ml of solution; assay gave 188.4 g product. The mother liquors were concentrated to 117 g of an oil. Proton NMR showed about 55% product, and 45% impurities.

The total yield (crystals filtered from reaction mixture and solids dissolved from the reactor surface) provided 988.6 g, 2.68 mols, 83.1% of compound 14-B.

The product (988.6 g compound 14-B) was used in the next reaction without any further purification.

### Step 3B: Synthesis of (2S,3S,4S,5R)-3-(3,4-difluoro-2-methoxyphenyl)-4,5-dimethyl-5-(trifluoromethyl)tetrahydrofuran-2-yl4-nitrobenzoate (14-A)

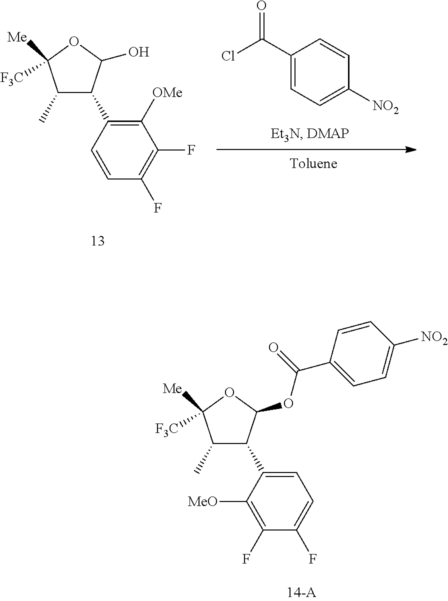 [SMILES: COc1c([C@H]2C(O)O[C@@](C)(C(F)(F)F)[C@H]2C)ccc(F)c1F ; O=C(Cl)c1ccc([N+](=O)[O-])cc1 ; COc1c([C@H]2[C@H](OC(=O)c3ccc([N+](=O)[O-])cc3)O[C@@](C)(C(F)(F)F)[C@H]2C)ccc(F)c1F]

### Procedure 1:

Compound 13 in a solution of 5 vol toluene was charged to the reactor. An additional 5 vol of toluene was charged bringing the total volume to 10 vol. Temperature was adjusted to 20±5° C. 4-nitrobenzoyl chloride was charged to the reactor as a solid. Temperature was adjusted to 0±5° C. Triethylamine was charged slowly to maintain temperature at 0±5° C. Temperature was adjusted to 20±5° C. and reaction mixture was stirred for no less than 3 h. Upon reaction completion, 5 vol of a 2 M aqueous NaOH solution was added to the reactor, maintaining a temperature of 20±5° C., and the biphasic mixture was stirred for no less than 1 hour. Agitation was stopped, the phases were allowed to settle and the bottom, aqueous phase was drained off. A 5 vol solution of saturated ammonium chloride was added the reactor and the biphasic mixture was stirred for no less than 30 minutes. Agitation was stopped, the phases were allowed to settle and the bottom, aqueous phase was drained off. A 5 vol solution of saturated ammonium chloride was added to the reactor and the biphasic mixture was stirred for no less than 30 minutes. 5 vol of water was charged to the reactor and the biphasic mixture was stirred for no less than 30 minutes. Phases were allowed to settle and the bottom, aqueous layer was drained off.

Organic phase was distilled to a total of 5 vol while maintaining an internal temperature at or below 45° C. 5 vol of toluene was charged and the mixture was distilled to 2.2 volumes. 1.2 vol of n-heptane was charged to the distilled toluene solution. Mixture was heated to an internal temperature of 70±5° C. and stirred for no less than 15 minutes and no more than 1 hour. Solution was cooled to 60±5° C. over 30 minutes. The clear solution was seeded with 0.010 w/w equiv of Compound 14-A seeds and agitated for no less than 1 hour and no more than 2 h. 2.4 vol of n-heptane was charged at a linear rate over 5 hours. Slurry was cooled to 20±5° C. over 5 h. Slurry was aged for no less than 5 h. The solids were isolated via filtration. A 2 vol wash solution of 75:25 n-heptane:toluene was used to wash the wet cake. The wet cake was transferred to drying equipment and dried under vacuum at a temperature of 40±5° C. until a constant weight was observed. The proton NMR of the crude material was consistent with the 4-nitrobenzoate ester of compound 14, compound 14-A. The total yield from Step 2 to Step 4B is 65%.

¹H-NMR CDCl3: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3H); 0.86-0.84 (d, 3H, J=8 Hz) ppm.

### Procedure 2:

Compound 13 (solution from above, 801 kg, 906.2 mol, 1.00 equiv) in a solution of 3 vol toluene was charged to the reactor. Temperature was adjusted to 20±5° C. Triethylamine (110.0 kg, 1087.4 mol, 1.20 equiv.) was charged, followed by 4-dimethylaminopyridine (DMAP, 1.66 kg, 13.6 mol, 0.015 equiv, 0.006 w/w equiv). A solution of 4-nitrobenzoyl chloride (185.0 kg, 996.8 mol, 1.10 equiv.) in 6.Ov MTBE (1296.5 kg) was prepared, then charged to the reactor over no less than 1 hour at a temperature of 20±5° C. The reaction mixture was stirred for no less than 30 min. Upon reaction completion, 5 vol of a 2 M aqueous NaOH solution was added to the reactor, maintaining a temperature of 20±5° C., and the biphasic mixture was stirred for no less than 1 hour. Agitation was stopped, the phases were allowed to settle and the bottom, aqueous phase was drained off. A 5 vol solution of saturated ammonium chloride was added the reactor and the biphasic mixture was stirred for no less than 30 minutes. Agitation was stopped, the phases were allowed to settle and the bottom, aqueous phase was drained off. 5 vol of water was charged to the reactor and the biphasic mixture was stirred for no less than 30 minutes. Phases were allowed to settle and the bottom, aqueous layer was drained off.

Organic phase was distilled to a total of 3.45 vol while maintaining an internal temperature at or below 45° C. Toluene (2025 kg, 8.0 vol) was charged and the mixture was distilled to 3.45 volumes. n-Heptane (155.8 kg, 0.78 vol) was charged to the reactor and the mixture was heated to an internal temperature of 55±5° C. and stirred for no less than 30 minutes to fully dissolve. The solution was cooled to 47±3° C. over 30 minutes. The clear solution was seeded with Compound 14-A (2.92 kg, 0.010 w/w equiv) seeds and agitated for no less than 1 hour. n-Heptane (747 kg, 3.7 vol) was charged at a linear rate over 1 hours, and the slurry was aged for 2 hours. The slurry was cooled to 5±5° C. over 140 min, then aged for no less than 12 h. The solids were isolated via filtration. A 2.4 vol wash solution of 75:25 n-heptane:toluene was used to wash the wet cake. The wet cake was transferred to drying equipment and dried under vacuum at a temperature of 40±5° C. until a constant weight was observed. The proton NMR of compound was consistent with the 4-nitrobenzoate ester of compound X, compound 14-A.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3H); 0.86-0.84 (d, 3H, J=8 Hz) ppm.

### Recrystallization:

Compound 14-A (258 kg, 543.8 mol, 1.00 equiv.) was charged to a reactor. Acetone (1264.6 kg, 6.24 vol) and water (315.4 kg, 1.22 vol) were added and agitation started. The slurry was heated to an internal temperature of 45±5° C. to fully dissolve. The solution was cooled to 30±3° C. at no more than 0.5° C./min. The clear solution was seeded with Compound 14-A (1.29 kg, 0.0050 w/w equiv) seeds and agitated for no less than 2 hours. Water (224.9 kg, 0.87 vol) was charged at a linear rate over 2 hours, and the slurry was aged for 1 hour to grow the seed bed. The slurry was cooled to 20±3° C. at a linear rate of no more than 0.2° C./min, than aged for no less than 8 h. The solids were isolated via filtration. A 2.0 vol wash solution of 75:25 acetone:water was used to wash the wet cake. The wet cake was transferred to drying equipment and dried under vacuum at a temperature of 40±5° C. until a constant weight was observed. The proton NMR of the recrystallized compound was consistent with benzoate ester of compound 14-A. The total yield from Step 2 to Step 4B is 65%.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3H); 0.86-0.84 (d, 3H, J=8 Hz) ppm.

### Step 4A: Synthesis of (2R,3S,4S,5R)-3-(3,4-difluoro-2-methoxyphenyl)-4,5-dimethyl-5-(trifluoromethyl)tetrahydrofuran-2-carbonitrile (15)

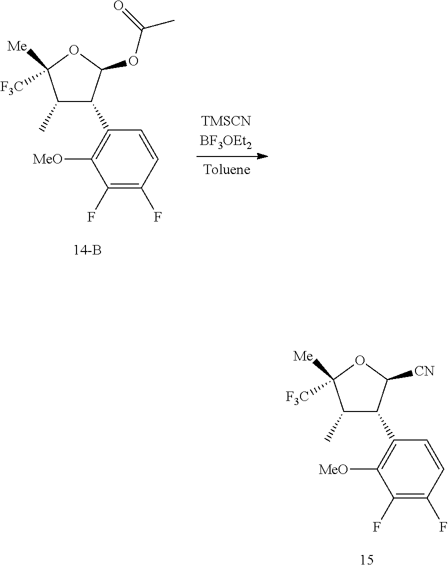 [SMILES: COc1c([C@H]2[C@H](OC(C)=O)O[C@@](C)(C(F)(F)F)[C@H]2C)ccc(F)c1F ; COc1c([C@H]2[C@H](C#N)O[C@@](C)(C(F)(F)F)[C@H]2C)ccc(F)c1F]

Transferred compound 14-B (1.87 L toluene solution containing 987 g of compound 14-B obtained in Step 4A; 2.68 mols) to a 50 L jacketed reactor predried with N₂ gas/vacuum 4 times under N₂ gas. Anhydrous toluene (7.0L) was added, stirred and cooled to −31° C. TMS-CN (385 g; 3.7 mols; 1.4 eq) was added over 8 minutes. BF₃ diethyl etherate (380 g=330 ml; 2.68 mols) was added dropwise over 6 minutes (Temp. −31.5° C. increased to −27.6° C.). The bath temperature was set at −21.6° C., and the reaction mixture was stirred for 2.5 hours. Potassium hydroxide (3.5 L of 2.0 M solution) was added over about 5 minutes (temperature rose to +8° C.). The bath temperature was raised to +20° C., the reaction mixture was stirred at +20° C. for about 10 minutes. The layers were separated and the aqueous layer was re-extracted with 6 L of toluene. The toluene solutions were re-extracted with 1.5 L of 2M potassium hydroxide before vacuum concentrating them to about 900 g of oil. The oil was diluted with 5 L of methanol and re-concentrated to give 887 g (2.65 mols, 98.7% is pure) of final crude compound IX. Proton NMR of the solid was consistent with the expected structure. NMR did not detect any methanol or toluene, suggesting that the solid is not solvated.

The crude compound 15 was purified using a preparative silica gel column packed in 85:15 hexane:MTBE. A solution of 0.78 kg of the crude product in the packing solvent mixture was transferred to the column and eluted with the packing solvent mixture. The fractions were split approximately in half and each half was reacted separately to prepare the next compound (compound 16). The early half of the fractions contained 333 g (or 993 mmols) of compound 15 whereas the late half of the fractions contained 274 g (or 818 mmols) of compound 15. The example below (i.e. Step 6) shows the process of converting compound 15 collected from the early half of the fractions. Compound IX obtained in the late half of the fractions was also converted to compound 16 following an identical process except for the amounts of reagents and solvents.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Step 4B: Synthesis of (2R,3S,4S,5R)-3-(3,4-difluoro-2-methoxyphenyl)-4,5-dimethyl-5-(trifluoromethyl)tetrahydrofuran-2-carbonitrile (15)

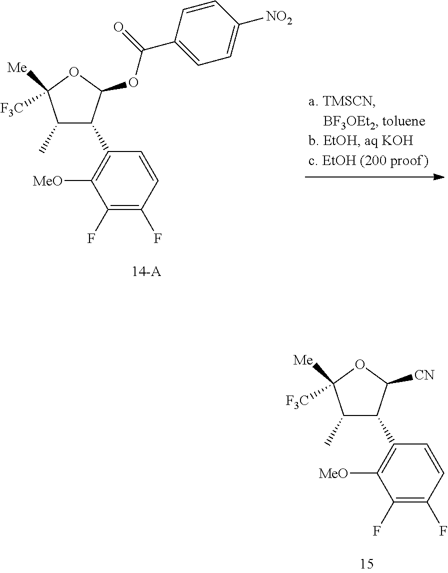 [SMILES: COc1c([C@H]2[C@H](OC(=O)c3ccc([N+](=O)[O-])cc3)O[C@@](C)(C(F)(F)F)[C@H]2C)ccc(F)c1F ; COc1c([C@H]2[C@H](C#N)O[C@@](C)(C(F)(F)F)[C@H]2C)ccc(F)c1F]

### Procedure 1:

Compound 14-A solids were charged to a reactor. 10 vol of toluene was charged to the reactor, and temperature was controlled to 20±5° C. 40% trimethylsilyl cyanide solution in toluene (1.2 equiv of TMSCN) was added to the reactor while maintaining a temperature of 20±5° C. Batch was cooled to −20±5° C. 1.0 equiv of boron trifluoride diethyl etherate was added slowly while maintaining the temperature at −20±5° C. Batch was stirred at −20±5° C. for no less than 3 hours. Upon reaction completion batch was heated back to 20±5° C. 10 vol of 20% w/w aqueous potassium hydroxide solution was added to the batch, the biphasic mixture was stirred for no less than 1 h. 5 vol of ethanol was added to the batch and the biphasic mixture was stirred for no less than 12 h. Phases were allowed to settle, and the bottom aqueous layer was drained off. 10 vol of 20% w/w aqueous potassium hydroxide solution was added to the batch, the biphasic mixture was stirred for no less than 1 h. Phases were allowed to settle, and the bottom aqueous layer was drained off. 10 vol of 20% w/w aqueous potassium hydroxide solution was added to the batch, the biphasic mixture was stirred for no less than 30 minutes. Phases were allowed to settle, and the bottom aqueous layer was drained off. 10 vol of water was added to the batch and the biphasic mixture was stirred for no less than 30 minutes. Phases were allowed to settle, and the bottom aqueous layer was drained off. Top, organic phase was distilled to a total of 4 vol while maintaining internal temperature at or below 45° C. 7 vol of ethanol was charged and the mixture was distilled to a total of 4 vol. Another 7 vol of ethanol was charged and the mixture was distilled to a total of 4 vol. Distillation continued until residual toluene was no more than 1.0% w/w. The proton NMR of the crude product was consistent with compound 15.

¹H-NMR CDCl3: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Procedure 2:

Compound 14-A solids (119.4 kg, 251.1 mol, 1.00 equiv.) were charged to a reactor. Toluene (517 kg, 5.0 vol) was charged to the reactor, and temperature was controlled to 20±5° C. 40% trimethylsilyl cyanide solution in toluene (376.8 mol, 1.5 equiv of TMSCN) was added to the reactor while maintaining a temperature of 20±5° C. Batch was cooled to −20±5° C. Boron trifluoride diethyl etherate (17.8 kg, 125.6 mol, 0.50 equiv.) was added slowly while maintaining the temperature at −20±5° C. Batch was stirred at −20±5° C. for no less than 3 hours. Upon reaction completion batch was heated back to 20±5° C. 10 vol of 20% w/w aqueous potassium hydroxide solution was added to the batch, the biphasic mixture was stirred for no less than 1 h. 5 vol of ethanol was added to the batch and the biphasic mixture was stirred for no less than 12 h. Phases were allowed to settle, and the bottom aqueous layer was drained off. 10 vol of 20% w/w aqueous potassium hydroxide solution was added to the batch, the biphasic mixture was stirred for no less than 1 h. Phases were allowed to settle, and the bottom aqueous layer was drained off. 10 vol of 20% w/w aqueous potassium hydroxide solution was added to the batch, the biphasic mixture was stirred for no less than 30 minutes. Phases were allowed to settle, and the bottom aqueous layer was drained off. 10 vol of water was added to the batch and the biphasic mixture was stirred for no less than 30 minutes. Phases were allowed to settle, and the bottom aqueous layer was drained off. Top, organic phase was distilled to a total of 4 vol while maintaining internal temperature at or below 45° C. 7 vol of ethanol was charged and the mixture was distilled to a total of 4 vol. Another 7 vol of ethanol was charged and the mixture was distilled to a total of 4 vol. Distillation continued until residual toluene was no more than 1.0% w/w. The proton NMR of the crude product was consistent with compound 15.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Procedure 3:

Compound 14-A solids (1.00 equiv) were charged to a reactor. Toluene (5.0 vol, 4.34 w/w equiv) was charged to the reactor, and temperature was controlled to 20±5° C. 40% trimethylsilyl cyanide solution in toluene (1.55 equiv, 0.32 w/w equiv) was added to the reactor while maintaining a temperature of 20±5° C. Batch was cooled to −20±5° C. Boron trifluoride diethyl etherate (1.00 equiv, 0.30 w/w equiv) was added over no less than 1.0 h while maintaining the temperature at −20±5° C. Batch was stirred at −20±5° C. for no less than 1.0 h and no more than 1.5 h. Acetone (1.00 equiv, 0.122 w/w equiv) was charged to quench the reaction while maintaining the temperature at −20±5° C. The batch was heated back to 10±5° C. and 10 vol of 5.9% w/w aqueous potassium hydroxide solution was added while the temperature was allowed to rise to 20±5° C. The biphasic mixture was stirred for no less than 2.0 h, then the phases were allowed to settle, and the bottom aqueous layer was drained off. 5.0 vol of 11.3% w/w aqueous potassium hydroxide solution was added to the batch, followed by ethanol (2.0 vol, 1.58 w/w equiv), and the biphasic mixture was stirred for no less than 2.0 h. Phases were allowed to settle, and the bottom aqueous layer was drained off. 5.0 vol of 11.3% w/w aqueous potassium hydroxide solution was added to the batch, followed by ethanol (2.0 vol, 1.58 w/w equiv), and the biphasic mixture was stirred for no less than 2.0 h. Phases were allowed to settle, and the bottom aqueous layer was drained off. The resulting organic layer was filtered through celite before being concentrated down to a total of 4.0 vol while maintaining internal temperature at or below 45° C. Ethanol (7.0 vol, 5.52 w/w equiv) was charged and the mixture was distilled to a total of 4.0 vol. Ethanol (7.0 vol, 5.52 w/w equiv) was charged and the mixture was distilled to a total of 4.0 vol. Distillation continued until residual toluene was no more than 5.0% w/w. The proton NMR of the crude product was consistent with compound 15.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Step 5: Synthesis of (2R,3S,45,5R)-3-(3,4-difluoro-2-methoxyphenyl)-4,5-dimethyl-5-(trifluoromethyl)tetrahydrofuran-2-carboxylic acid (16)

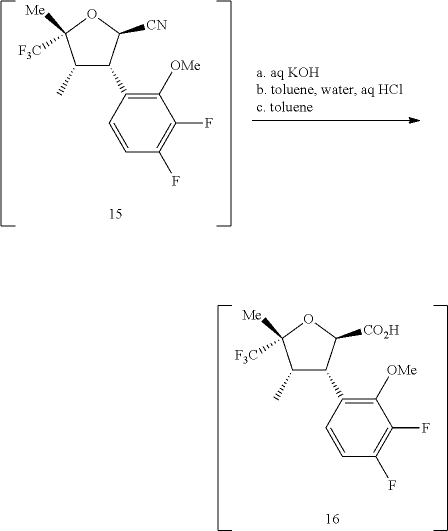

### Procedure 1:

Compound 15 (333.06 g, 993.4 mmol) was dissolved in 2.60 L methanol and 2.60 L of 2.OM potassium hydroxide in a 25 L jacketed reactor under N₂ gas. The mixture was stirred at 55° C. for 20 hours. After overnight the slurry was a clear solution. Proton NMR indicated that the reaction complete. The reaction mixture was cooled to +15° C., and added 2925 ml of 2N HCl+5 L MTBE. After stirring for about 5 minutes, the layers were separated, and the aqueous was re-extracted with 5 L of MTBE. The MTBE solutions were combined and dried over magnesium sulfate, filtered and washed with MTBE. The combined MTBE solution was concentrated to an oil and re-concentrated with 4 L toluene, to give 360 g of an oily product, compound 16.

### Procedure 2:

To compound 15 in 4 vol ethanol (245.3 kg, 168.3 mol, 1.00 equiv.) at 20° C., was charged a 10% w/w aqueous solution of KOH (192.8 kg, 2.4 vol, 2.38 equiv KOH). The reaction mixture was heated to 55° C. and stirred or 12 h. Upon completion of the reaction, the mixture was cooled down to 20° C. and toluene (242.8 kg, 3.5 vol) and water (400.0 kg, 5.0 vol) were charged. After 30 min of stirring, the phases were separated, and the organic phase was discarded. The aqueous phase was extracted with toluene (381.5 kg, 5.5 vol) and 7% HCl solution (537.2 kg, 6.5 vol). After 60 min of stirring the phases were separated and the aqueous phase was discarded. The organic phase was washed with water (400.0 kg, 5.0 vol), twice. The organic phase was distilled down to 4.0 volumes under vacuum at an internal temperature below 40° C. Toluene (277.4 kg, 4.0 vol) was charged and the solution was distilled down to 4.0 volumes under vacuum at an internal temperature below 40° C., to afford compound 16. The proton NMR of the crude compound was consistent with compound 16.

¹H-NMR d6-DMSO: δ 13.00 (s, 1H); 7.27-7.08 (m, 2H); 4.98 (d, 1H, J=10.5 Hz); 4.08 (dd, 1H, J=10.5, 7.6 Hz); 3.93 (d, 3H, J=2.1 Hz); 2.66 (p, 1 H, J=7.5 Hz); 1.53 (d, 3H, J=1.4 Hz); 0.73-0.64 (m, 3H) ppm.

### Step 6: Synthesis of (R)-(+)-α-methylbenzylamme salt of (2R,35,45,5R)-3-(3,4-difluoro-2-methoxyphenyl)-4,5-dimethyl-5-(trifluoromethyl)tetrahydrofuran-2-carboxylic acid (17)

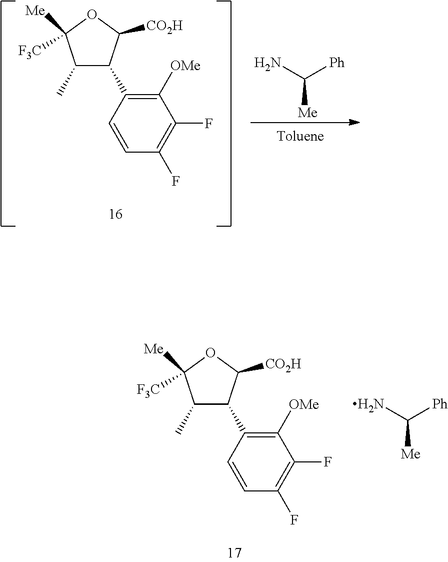

### Procedure 1:

Compound 16 (2 g) was dissolved in 10 mL of toluene at 60° C. Separately, (R)-AMB (1.2 eq.) was dissolved in 2 mL of toluene and added to Compound 16 solution over 1 hour. The resulting solution was then cooled to 50° C. over 1 hour and held for 1 hour for the self-nucleation to happen. Then the slurry was cooled to 20° C. over 5 hours and agitated at 20° C. for about 8 hours and filtered under vacuum. The resulting wet cake was washed with a 5 mL of toluene and dried under vacuum at 40° C. to provide about 70% yield compared to input Compound 16 free form. The resulting solid was identified as (R)-(+)-α-methylbenzylamine salt of Compound 16 Form A from XRPD, i.e. Form A of Compound 17.

Form A of Compound 17 presents the following XRPD peaks in a standard X-Ray Powder Diffraction experiment:

| No. | Pos. [°2θ] | Rel. Int. [%] |
|---|---|---|
| 1 | 10.32 | 28.04 |
| 2 | 11.78 | 11.26 |
| 3 | 12.81 | 15.58 |
| 4 | 13.44 | 57.18 |
| 5 | 14.01 | 5.72 |
| 6 | 14.76 | 6.18 |
| 7 | 15.91 | 18.58 |
| 8 | 17.00 | 5.45 |
| 9 | 17.45 | 100 |
| 10 | 18.25 | 36.72 |
| 11 | 18.53 | 11.02 |
| 12 | 18.95 | 63.98 |
| 13 | 21.96 | 11.86 |
| 14 | 24.07 | 24.65 |
| 15 | 24.36 | 8.98 |
| 16 | 24.62 | 7.83 |
| 17 | 24.69 | 7.32 |
| 18 | 25.09 | 8.2 |
| 19 | 29.69 | 7.8 |

### Procedure 2:

The previously prepared solution of compound 16 in 4.0 vol toluene (445.7 kg, 250.3 mol, 1.00 equiv) was charged to a reactor and the temperature was adjusted to 20±5° C. Separately, (R)-α-methylbenzylamine ((R)-AMB, 36.4 kg, 300.4 mol, 1.2 equiv.) was dissolved in toluene (51.6 kg, 0.50 vol). 40 vol % of the previously prepared solution was charged to the compound 16 solution over 1 hour maintaining the temperature at 20±5° C., then the solution was stirred for no less than 60 minutes. Compound 16 seeds (0.30 kg, 0.0025 w/w equiv.) were charged to the solution, and the slurry was stirred for no less than 1 hour. The remaining 60 vol % of the (R)-AMB solution was then charged to the slurry over no less than 1 hour, maintaining the temperature at 20±5° C. Then the slurry was aged at 20±5° C. for no less than 8 hours and filtered. The resulting wet cake was washed with toluene (206.3 kg, 2.0 vol) and dried under vacuum at 40° C. The resulting solid was identified as (R)-(+)-α-methylbenzylamine salt of Compound 16 Form A from XRPD, i.e. Form A of Compound 17. The total yield from Step 5 to Step 7 is 80%.

Compound 21 can be prepared from Compound 17 by the process described below:

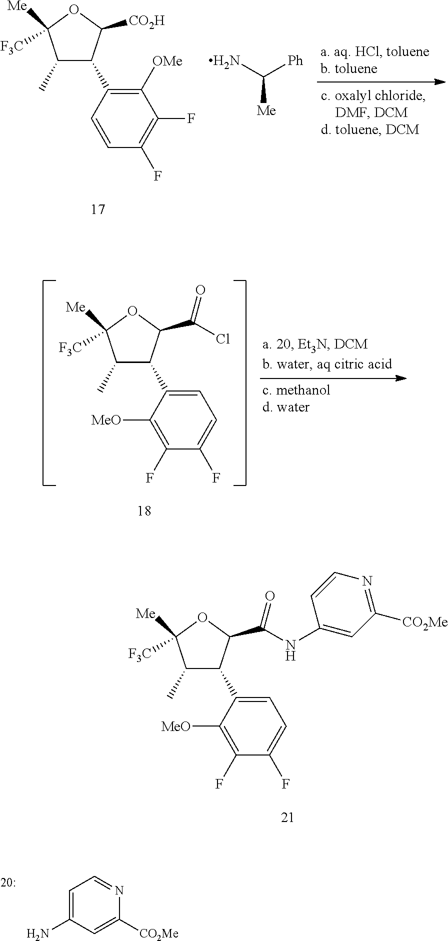

### Procedure 1:

Compound 17 (205.6 kg, 432.4 mol, 1.00 equiv.) was dissolved with toluene (1070 kg, 6.0 vol) into a reactor and the temperature was adjusted to 20±5° C. A 2 M aqueous hydrochloric acid solution (637 kg, 3.0 vol) was charged, and the biphasic mixture was stirred for 30 minutes. The aqueous phase was separated out, then a 2 M aqueous hydrochloric acid solution (637 kg, 3.0 vol) was charged to the organic phase and the biphasic mixture was stirred for 30 minutes. The aqueous phase was separated out, then water (411.2 kg, 2.0 vol) was charged to the organic phase and the biphasic mixture was stirred for 30 minutes.

The resulting emulsion was phase separated and the organic phase was distilled down to 3.0 vol under vacuum at a maximum internal temperature of 45° C. Toluene (1070 kg, 6.0 vol) was charged and the solution was distilled down to 3.0 vol under vacuum at a maximum internal temperature of 45° C.

Dichloromethane (DCM, 817.3 kg, 3.0 vol) was charged to the previous solution, followed by N,N-dimethylformamide (DMF, 0.47 kg, 6.5 mol, 0.015 equiv.), and the resulting mixture was heated to 30±5° C. A previously prepared solution of oxalyl chloride (71.3 kg, 562.2 mol, 1.30 equiv.) in DCM (272.4 kg, 1.0 vol) was charged to the reaction mixture over no less than 1 hour while maintaining the temperature at 30±5° C. Reaction was allowed to progress for over 3 hours to form Compound 18.

Post reaction, a series of put-take distillation cycles were performed (chasing with 10 vol toluene then 10 vol dichloromethane) to remove residual oxalyl chloride, resulting in a 3.0 vol solution of Compound 18 in dichloromethane. Dichloromethane (544.8 kg, 2.0 vol) was charged to make it a 5.0 vol solution.

Compound 20 (72.2 kg, 474.3 mol, 1.10 equiv.) was slurried into DCM (1086.5 kg, 4.0 vol) along with triethylamine (TEA, 52.4 kg, 517.4 mol, 1.20 equiv.) at a temperature of 25±5° C. The previous 5.0 vol solution of Compound 18 was charged in no less than 60 minutes, maintaining the temperature at 25±5° C., then the reaction mixture was stirred for no less than 1 hour.

After reaction completion, water (820.0 kg, 4.0 vol) was charged, the biphasic mixture was stirred for 30 minutes, allowed to settle for 30 min, then the aqueous phase was discarded. A 1 M citric acid aqueous solution (3.0 vol) was charged to the organic phase, the biphasic mixture was stirred for 30 minutes, allowed to settle for 30 min, then the aqueous phase was discarded. Water (410.0 kg, 2.0 vol) was charged, the biphasic mixture was stirred for 30 minutes, allowed to settle for 30 min, then the aqueous phase was discarded.

Solvent swap was performed via put-take distillation to exchange toluene with methanol, resulting in a 4.0 vol solution of Compound 21 in methanol. A pre-mixed mixture of methanol (569.9 kg, 3.51 vol) and water (147.6 kg, 0.72 vol) was added to the latter solution. The batch was heated to 50±3° C. to fully dissolve. The batch was cooled to 40±3° C. before Compound 21 seeds (1.03 kg, 0.0050 w/w equiv.) were added, and the seed bed was allowed to grow for 60 min at 40±3° C. Water (440.8 kg, 2.15 vol) was charged over no less than 3 hours, and the slurry was stirred at 40±3° C. for 30 min before being cooled to 20±3° C. over 5 hours and aged for 8 hours. The slurry was filtered and the resulting wet cake was washed with a previously prepared solution of methanol (324.7 kg, 2.0 vol) and water (176.3 kg, 0.86 vol) before drying at 40° C. to provide Compound 21. The isolated form is Form C in 85% yield.

### Procedure 2:

Compound 17 (1.00 equiv) was slurried with toluene (6.0 vol, 5.19 w/w equiv) into a reactor and the temperature was adjusted to 20±5° C. A 2 M aqueous hydrochloric acid solution (3.0 vol) was charged, and the biphasic mixture was stirred for 30 minutes. The aqueous phase was separated out, then a 2 M aqueous hydrochloric acid solution (3.0 vol) was charged to the organic phase and the biphasic mixture was stirred for 30 minutes. The aqueous phase was separated out, then water (2.0 vol) was charged to the organic phase and the biphasic mixture was stirred for 30 minutes.

The resulting emulsion was phase separated and the organic phase was distilled down to 3.0 vol under vacuum at a maximum internal temperature of 45° C. Toluene (6.0 vol, 5.19 w/w equiv) was charged and the solution was distilled down to 3.0 vol under vacuum at a maximum internal temperature of 45° C.

Toluene (3.0 vol, 2.60 w/w equiv) was charged to the previous solution, followed by N,N-dimethylformamide (DMF, 0.015 equiv, 0.0023 w/w equiv), and the resulting mixture was heated to 30±5° C. A previously prepared solution of oxalyl chloride (1.30 equiv, 0.35 w/w equiv) in toluene (1.0 vol, 0.87 w/w equiv) was charged to the reaction mixture over no less than 1 hour while maintaining the temperature at 30±5° C. Reaction was allowed to progress for over 3 hours to form Compound 18.

Post reaction, a series of put-take distillation cycles were performed (chasing with 10 vol toluene) to remove residual oxalyl chloride, resulting in a 3.0 vol solution of Compound 18 in toluene. Toluene (2.0 vol, 1.73 w/w equiv) was charged to make it a 5.0 vol solution.

Compound 20 (1.10 equiv, 0.35 w/w equiv) was slurried into toluene (4.0 vol, 3.47 w/w equiv) along with triethylamine (TEA, 1.20 equiv, 0.26 w/w equiv) at a temperature of 25±5° C. The previous 5.0 vol solution of Compound 18 was charged in no less than 1.0 h, maintaining the temperature at 20±5° C., then the reaction mixture was stirred for no less than 2.0 hour.

After reaction completion, ethyl acetate (EtOAc, 3.0 vol, 2.71 w/w equiv) and water (4.0 vol, 4.00 w/w equiv) were charged, the biphasic mixture was stirred for 30 minutes, allowed to settle for 30 min, then the aqueous phase was discarded. A 1 M citric acid aqueous solution (3.0 vol) was charged to the organic phase, the biphasic mixture was stirred for 30 minutes, allowed to settle for 30 min, then the aqueous phase was discarded. Water (2.0 vol) was charged, the biphasic mixture was stirred for 30 minutes, allowed to settle for 30 min, then the aqueous phase was discarded.

Solvent swap was performed via put-take distillation to exchange toluene and EtOAc with methanol, resulting in a 3.0 vol solution of Compound 21 in methanol.

Methanol (4.5 vol, 3.56 w/w equiv) and water (0.95 vol, 0.95 w/w equiv) were added to the latter solution. The batch was heated to 60±3° C. to fully dissolve. The batch was cooled to 40±3° C. before Compound 21 seeds (0.0050 w/w equiv) were added, and the seed bed was allowed to grow for 2.0 h at 40±3° C. Water (2.3 vol, 2.30 w/w equiv) was charged over no less than 5 hours, and the slurry was cooled to 20±3° C. over 2 hours and aged for no less than 1.0 h. The slurry was filtered and the resulting wet cake was washed with a previously prepared solution of methanol (2.8 vol, 2.22 w/w equiv) and water (1.2 vol, 1.20 w/w equiv) before drying at 45±5° C. to provide Compound 21. The isolated form is Form C in 85% yield.

Compound 1 can be prepared from Compound 21 by the process described below:

### Step 1: Synthesis of Compound 1

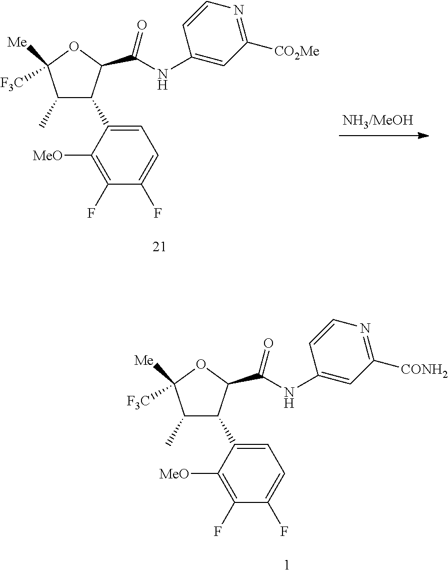 [SMILES: COC(=O)c1cc(NC(=O)[C@@H]2O[C@@](C)(C(F)(F)F)[C@@H](C)[C@H]2c2ccc(F)c(F)c2OC)ccn1 ; COc1c([C@H]2[C@H](C(=O)Nc3ccnc(C(N)=O)c3)O[C@@](C)(C(F)(F)F)[C@H]2C)ccc(F)c1F]

### Procedure 1:

Compound 21 (1.6 kg) was charged to a 100 L reactor followed by a 7 M solution of ammonia in methanol (10.7 kg). The temperature of the mixture was adjusted to 20±5° C. The mixture was agitated at 20±5° C. for at least 24 h or until the amount of the starting material present was <0.5% (an additional amount of ammonia solution may be added if necessary to reduce compound 21 to below 0.5%). The reaction mixture was transferred to a rotovap and distilled to 4 vol. Methanol was charged to the rotovap rbf and the mixture was distilled to 4 vol. The mixture was transferred to the 100 L reactor and methanol was charged. The temperature was adjusted to 55±5° C. and the mixture was agitated at 55±5° C. for at least 10 min. If a solution was not present in the reactor, the temperature of the mixture was adjusted to 60±5° C. and the mixture was agitated for at least 10 min followed by adjusting the temperature to 55±5° C. Water (7.9 kg) was charged over at least 2 hours to the rector while maintaining the temperature at 55±5° C. The mixture was agitated at 55±5° C. for at least 1 h followed by adjustment of the temperature to 20±5° C. over at least 12 h. The mixture was agitated at 20±5° C. for at least 5 hours and the solids were collected by filtration. The reactor was rinsed with methanol/water and the rinse was transferred through the filter cake. The solids were transferred to lined trays and dried in a tray drier at <45° C. for at least 12 h to yield 1.3 kg of crude Compound 1 in 80% yield.

### Procedure 2:

Ammonia gas was bubbled into a 30 vol % tetrahydrofuran (385.8 kg, 3.10 vol) in methanol (798.3 kg, 7.20 vol) solution to result in about 6 M concentrated solution. Compound 21 (140.0 kg, 286.6 mol, 1.00 equiv.) was charged to a sealed reactor, followed by the previously prepared ammonia solution, and solution was stirred at 20±5° C. for no less than 20 hours. The resulting Compound 1 solution was heated to 25±5° C., then water (175.0 kg, 1.25 vol) was charged over 1 hour at 25±5° C. Seeding was carried with Compound 1 crystals (0.70 kg, 0.0050 w/w equiv.) at 25 ±3° C. and aged for 1 hour. Water (1225.0 kg, 8.75 vol) was charged at 25±5° C. for over 4.5 hours. The slurry was aged for over 8 hours and filtered under vacuum. The resulting wet cake was washed with a methanol/tetrahydrofuran/water (35/15/50 volume ratio) and dried under vacuum at 40° C. to provide about 92 to 94% yield of Compound 1. The isolated form is Form B in 90-93% yield.

### Procedure 3:

Ammonia was charged into a 30 vol % tetrahydrofuran (THF 3.0 vol, 2.67 w/w equiv) in methanol (7.0 vol, 5.54 w/w equiv) solution to result in no less than 5.2 M solution. Compound 21 (1.00 equiv) was charged to a sealed reactor, followed by the previously prepared ammonia solution, and solution was stirred at 20±5° C. for no less than 24 hours.

The resulting Compound 1 solution was cooled to 18±3° C., then water (1.25 vol, 1.25 w/w equiv) was charged over 1 hour at 18±3° C. Compound 1 seeds (0.0050 w/w equiv) were charged at 18±3° C. and aged for 1 hour. Water (8.75 vol, 8.75 w/w equiv) was charged at 18±3° C. for over 4.5 hours. The slurry was aged for no less than 1 h and filtered. The resulting wet cake was washed with a previously prepared solution of methanol (1.05 vol, 0.83 w/w equiv), THF (0.45 vol, 0.40 w/w equiv), and water (1.50 vol, 1.50 w/w equiv), then dried under vacuum at 50±5° C. to provide dry Compound 1. The isolated form is Form B in 92-94% yield.

### Step 2: Recrystallization of Compound 1

### Procedure 1:

Crude Compound 1 (1.23 kg) was charged to a 22 L rbf followed by acetone (5.01 kg). The mixture was agitated and the temperature was adjusted to 40±5° C. The mixture was polish filtered to a 20 L Jacketed Reactor. The temperature was adjusted to 35±5° C. and the mixture was agitated at 35±5° C. for at least 5 min. If the mixture was not a solution, the temperature was adjusted to 40±5° C. and the mixture was agitated at 40±5° C. for at least an additional 5 min. The temperature was adjusted to 35±5° C. and water (1.90 kg) was charged to the solution over the course of 1.5 h before seeding the solution. The mixture was agitated at 35±5° C. for at least 1 h. Water (2.49 kg) was charged to the reactor over at least 2 hours while maintaining a temperature of 35±5° C. and the mixture was agitated at 35±5° C. for at least 30 min. The temperature was adjusted to 20±5° C. over at least 5 h and the mixture was agitated at 20±5° C. for at least 5 h. The solid was collected by filtration and the reactor was rinsed with acetone/water sending the rinse through the filter cake. The solid was dried on the filter with N₂ gas for at least 30 min. The product was transferred to lined trays and dried in a tray drier to yield 1.10 kg of recrystallized Compound 1.

### Procedure 2:

Compound 1 (120.0 kg, 253.5 mol, 1.00 equiv.) was charged to a reactor followed by methanol (665.3 kg, 7.0 vol) and tetrahydrofuran (320.0 kg, 3.0 vol) at 25±5° C. Polish filtration was then conducted. Water (150.0 kg, 1.25 vol) was charged over 1 hour at 25±3° C. Seeding was carried out with Compound 1 crystals (0.60 kg, 0.0050 w/w equiv.) at 25±3° C. and aged for 1 hour. Water (1050.0 kg, 8.75 vol) was charged at 25±3° C. for over 4.5 hours. The slurry was aged for over 8 hours and filtered under vacuum. The resulting wet cake was washed with a methanol/tetrahydrofuran/water (35/15/50 volume ratio) and dried under vacuum at 40° C. to provide about 95% yield of Compound 1. The isolated form was Form B.

Many modifications and variations of the embodiments described herein may be made without departing from the scope, as is apparent to those skilled in the art. The specific embodiments described herein are offered by way of example only.

What is claimed is:

1. A method of treating chronic pain in a subject, comprising administering to the subject Compound 1: 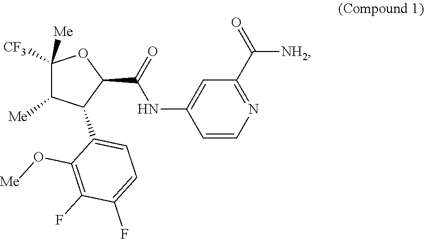 [SMILES: COc1c([C@H]2[C@H](C(=O)Nc3ccnc(C(N)=O)c3)O[C@@](C)(C(F)(F)F)[C@H]2C)ccc(F)c1F]
or a pharmaceutically acceptable salt thereof, in an amount of 15-80 mg per day.

2. The method of claim 1, wherein Compound 1, or a pharmaceutically acceptable salt thereof, is administered in an amount of about 70 mg per day.

3. The method of claim 2, wherein the chronic pain is moderate to severe chronic pain.

4. The method of claim 3, wherein the chronic pain is neuropathic pain, musculoskeletal pain, or visceral pain.

5. The method of claim 4, wherein the chronic pain is diabetic peripheral neuropathy.

6. The method of claim 4, wherein the chronic pain is lumbosacral radiculopathy.

7. The method of claim 5, wherein the subject experiences (a) a change of −1 to −4 in weekly average of daily pain on an 11-point Numeric Pain Rating Scale (NPRS) at week 12 of treatment, relative to baseline; or (b) an improvement in sleep, as determined by a change in Daily Sleep Interference Scale (DSIS) score at week 12 of treatment, relative to baseline.

8. The method of claim 1, wherein Compound 1 is administered in a pharmaceutical composition comprising: 45-55 wt % of a solid dispersion comprising a polymer and Compound 1; 40-50 wt % of one or more fillers; 2-4 wt % of a disintegrant; 0.5-1.5 wt % of a lubricant; and 0.05-0.8 wt % of a glidant.

9. The method of claim 1, wherein Compound 1 is administered in a pharmaceutical composition comprising:
about 50 wt % of a solid dispersion comprising HPMCAS and Compound 1, wherein the solid dispersion comprises about 75 wt % of HPMCAS and about 25 wt % of Compound 1;
about 22.9 wt % of microcrystalline cellulose;
about 22.9 wt % of mannitol;
about 3 wt % of croscarmellose sodium;
about 1.0 wt % of magnesium stearate; and
about 0.2 wt % of colloidal silicon dioxide.

10. The method of claim 1, wherein Compound 1 is administered in a pharmaceutical composition comprising:
about 280 mg of a solid dispersion comprising HPMCAS and Compound 1, wherein the solid dispersion comprises about 210 mg of HPMCAS and about 70 mg of Compound 1;
about 128.2 mg of microcrystalline cellulose;
about 128.2 mg of mannitol;
about 16.8 mg of croscarmellose sodium;
about 5.6 mg of magnesium stearate; and
about 1.1 mg of colloidal silicon dioxide.

11. The method of claim 10, wherein the pharmaceutical composition is a tablet core composition.

12. A method of treating acute pain in a subject, comprising administering to the subject Compound 1: 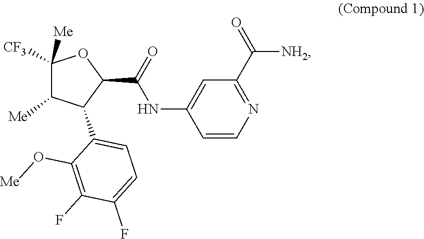 [SMILES: COc1c([C@H]2[C@H](C(=O)Nc3ccnc(C(N)=O)c3)O[C@@](C)(C(F)(F)F)[C@H]2C)ccc(F)c1F]
or a pharmaceutically acceptable salt thereof, in a first dose of 85-115 mg, followed by subsequent doses of 40-60 mg every 12 hours.

13. The method of claim 12, wherein the first dose is about 100 mg, and the subsequent doses are about 50 mg.

14. The method of claim 13, wherein the acute pain is moderate to severe acute pain.

15. The method of claim 14, wherein the acute pain is acute post-operative pain or postsurgical pain.

16. The method of claim 15, wherein the acute pain is bunionectomy pain.

17. The method of claim 15, wherein the acute pain is abdominoplasty pain.

18. The method of claim 16, wherein the subject experiences a difference from placebo in time-weighted sum of pain intensity difference, relative to baseline, as recorded on an 11-point Numeric Pain Rating Scale (NPRS), from 0 to 48 hours (SPID48) of 10.0 to 50.0.

19. The method of claim 17, wherein the subject experiences a difference from placebo in time-weighted sum of pain intensity difference, relative to baseline, as recorded on an 11-point Numeric Pain Rating Scale (NPRS), from 0 to 48 hours (SPID48) of 30.0 to 65.0.

20. The method of claim 12, wherein Compound 1 is administered in a pharmaceutical composition comprising: 45-55 wt % of a solid dispersion comprising a polymer and Compound 1; 42-50 wt % of one or more fillers; 2-4 wt % of a disintegrant; and 0.5-1.5 wt % of a lubricant.

21. The method of claim 12, wherein Compound 1 is administered in a pharmaceutical composition comprising:
about 50 wt % of a solid dispersion comprising HPMCAS and Compound 1, wherein the solid dispersion comprises about 75 wt % of HPMCAS and about 25 wt % of Compound 1;
about 46 wt % of microcrystalline cellulose;
about 3 wt % of croscarmellose sodium; and
about 1.0 wt % of magnesium stearate.

22. The method of claim 21, wherein Compound 1 is administered in a pharmaceutical composition comprising:
about 200 mg of a solid dispersion comprising HPMCAS and Compound 1, wherein the solid dispersion comprises about 150 mg of HPMCAS and about 50 mg of Compound 1;
about 184 mg of microcrystalline cellulose;
about 12 mg of croscarmellose sodium; and
about 4 mg of magnesium stearate.

23. The method of claim 22, wherein the pharmaceutical composition is a tablet core composition.

24. A pharmaceutical composition comprising: 45-55 wt % of a solid dispersion comprising a polymer and Compound 1; 40-50 wt % of one or more fillers; 2-4 wt % of a disintegrant; 0.5-1.5 wt % of a lubricant; and 0.05-0.8 wt % of a glidant.

25. The pharmaceutical composition of claim 24, wherein the pharmaceutical composition comprises:
about 50 wt % of a solid dispersion comprising HPMCAS and Compound 1, wherein the solid dispersion comprises about 75 wt % of HPMCAS and about 25 wt % of Compound 1;
about 22.9 wt % of microcrystalline cellulose;
about 22.9 wt % of mannitol;
about 3 wt % of croscarmellose sodium;
about 1.0 wt % of magnesium stearate; and
about 0.2 wt % of colloidal silicon dioxide.

26. The pharmaceutical composition of claim 24, wherein the pharmaceutical composition comprises:
about 280 mg of a solid dispersion comprising HPMCAS and Compound 1, wherein the solid dispersion comprises about 210 mg of HPMCAS and about 70 mg of Compound 1;
about 128.2 mg of microcrystalline cellulose;
about 128.2 mg of mannitol;
about 16.8 mg of croscarmellose sodium;
about 5.6 mg of magnesium stearate; and
about 1.1 mg of colloidal silicon dioxide.

27. A pharmaceutical composition comprising: 45-55 wt % of a solid dispersion comprising a polymer and Compound 1; 42-50 wt % of one or more fillers; 2-4 wt % of a disintegrant; and 0.5-1.5 wt % of a lubricant.

28. The pharmaceutical composition of claim 27, wherein the pharmaceutical composition comprises:
about 50 wt % of a solid dispersion comprising HPMCAS and Compound 1, wherein the solid dispersion comprises about 75 wt % of HPMCAS and about 25 wt % of Compound 1;
about 46 wt % of microcrystalline cellulose;
about 3 wt % of croscarmellose sodium; and
about 1.0 wt % of magnesium stearate.

29. The pharmaceutical composition of claim 27, wherein the pharmaceutical composition comprises:
about 200 mg of a solid dispersion comprising HPMCAS and Compound 1, wherein the solid dispersion comprises about 150 mg of HPMCAS and about 50 mg of Compound 1;
about 184 mg of microcrystalline cellulose;
about 12 mg of croscarmellose sodium; and
about 4 mg of magnesium stearate.

30. The pharmaceutical composition of claim 29, wherein the pharmaceutical composition is a tablet core composition.
